SEQUENCE LISTING (90 sequences)

SEQ ID NO: 1 - protein, 4 aa, Artificial Sequence
  an artificially synthesized sequence
GGGS

SEQ ID NO: 2 - protein, 4 aa, Artificial Sequence
  an artificially synthesized sequence
GGSG

SEQ ID NO: 3 - protein, 4 aa, Artificial Sequence
  an artificially synthesized sequence
GSGG

SEQ ID NO: 4 - protein, 4 aa, Artificial Sequence
  an artificially synthesized sequence
SGGG

SEQ ID NO: 5 - protein, 4 aa, Artificial Sequence
  an artificially synthesized sequence
GSSG

SEQ ID NO: 6 - protein, 5 aa, Artificial Sequence
  an artificially synthesized sequence
GGGGS

SEQ ID NO: 7 - protein, 5 aa, Artificial Sequence
  an artificially synthesized sequence
GGGSG

SEQ ID NO: 8 - protein, 5 aa, Artificial Sequence
  an artificially synthesized sequence
GGSGG

SEQ ID NO: 9 - protein, 5 aa, Artificial Sequence
  an artificially synthesized sequence
GSGGG

SEQ ID NO: 10 - protein, 5 aa, Artificial Sequence
  an artificially synthesized sequence
GSGGS

SEQ ID NO: 11 - protein, 5 aa, Artificial Sequence
  an artificially synthesized sequence
SGGGG

SEQ ID NO: 12 - protein, 5 aa, Artificial Sequence
  an artificially synthesized sequence
GSSGG

SEQ ID NO: 13 - protein, 5 aa, Artificial Sequence
  an artificially synthesized sequence
GSGSG

SEQ ID NO: 14 - protein, 5 aa, Artificial Sequence
  an artificially synthesized sequence
SGGSG

SEQ ID NO: 15 - protein, 5 aa, Artificial Sequence
  an artificially synthesized sequence
GSSSG

SEQ ID NO: 16 - protein, 6 aa, Artificial Sequence
  an artificially synthesized sequence
GGGGGS

SEQ ID NO: 17 - protein, 6 aa, Artificial Sequence
  an artificially synthesized sequence
SGGGGG

SEQ ID NO: 18 - protein, 7 aa, Artificial Sequence
  an artificially synthesized sequence
GGGGGGS

SEQ ID NO: 19 - protein, 7 aa, Artificial Sequence
  an artificially synthesized sequence
SGGGGGG

SEQ ID NO: 20 - protein, 6 aa, Artificial Sequence
  an artificially synthesized sequence
ASTKGP

SEQ ID NO: 21 - protein, 13 aa, Artificial Sequence
  an artificially synthesized sequence
ASTKGPSVFPLAP

SEQ ID NO: 22 - protein, 6 aa, Artificial Sequence
  an artificially synthesized sequence
GGGGSG

SEQ ID NO: 23 - protein, 10 aa, Artificial Sequence
  an artificially synthesized sequence
GGGGSGGGGS

SEQ ID NO: 24 - protein, 14 aa, Artificial Sequence
  an artificially synthesized sequence
GGGGSGGGGSGGGG

SEQ ID NO: 25 - protein, 6 aa, Artificial Sequence
  an artificially synthesized sequence
RTVAAP

SEQ ID NO: 26 - protein, 13 aa, Artificial Sequence
  an artificially synthesized sequence
RTVAAPSVFIFPP

SEQ ID NO: 27 - protein, 9 aa, Artificial Sequence
  an artificially synthesized sequence
GGSGGGGSG

SEQ ID NO: 28 - protein, 13 aa, Artificial Sequence
  an artificially synthesized sequence
GGSGGGGSGGGGS

SEQ ID NO: 29 - protein, 7 aa, Artificial Sequence
  an artificially synthesized sequence
GQPKAAP

SEQ ID NO: 30 - protein, 14 aa, Artificial Sequence
  an artificially synthesized sequence
GQPKAAPSVTLFPP

SEQ ID NO: 31 - protein, 465 aa, Artificial Sequence
  an artificially synthesized sequence
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGSGLNDIFEAQKIEWHE

SEQ ID NO: 32 - protein, 220 aa, Artificial Sequence
  an artificially synthesized sequence
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYYYHMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 220 aa, Artificial Sequence
  an artificially synthesized sequence
DLVMTQSPSSLSASVGDRVTITCKSSQSLLDSDDGNTYLAWYQQKPGKAPKLLIYWLSYRASGVPSRFSGSGSGTDFTLTISSLQPEDFATYYCQQYYYHMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 465 aa, Artificial Sequence
  an artificially synthesized sequence
QVQLQQPGAELVKPGASVKLSCKASGYTFTRFWINWVRQRPGQGLEWIGNIYPGSSSINYNEKFKNKATLTVDTSSSTAYMQLSSLTSDDSAVYYCARRRYRSSYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGSGLNDIFEAQKIEWHE

SEQ ID NO: 35 - protein, 214 aa, Artificial Sequence
  an artificially synthesized sequence
ETTVTQSPASLSVATGEKVTIRCITNTDIDDDMNWYQQKPGEPPKLLISEGNTLRPGVPSRFSSSGYGTDFVFTIENTLSEDVADYCCLQSDNMPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 464 aa, Artificial Sequence
  an artificially synthesized sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSPGGGGSGLNDIFEAQKIEWHE

SEQ ID NO: 37 - protein, 214 aa, Artificial Sequence [sequence omitted]

SEQ ID NO: 38 - protein, 464 aa, Artificial Sequence
  an artificially synthesized sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSPGGGGSGLNDIFEAQKIEWHE

SEQ ID NO: 39 - protein, 214 aa, Artificial Sequence
  an artificially synthesized sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 461 aa, Artificial Sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSPGGGGSGLNDIFEAQKIEWHE

SEQ ID NO: 41 - protein, 214 aa, Artificial Sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 461 aa, Artificial Sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGSGLNDIFEAQKIEWHE

SEQ ID NO: 43 - protein, 321 aa, Artificial Sequence
  an artificially synthesized sequence
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 44 - protein, 448 aa, Artificial Sequence
  an artificially synthesized sequence
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSP

SEQ ID NO: 45 - protein, 240 aa, Artificial Sequence
  an artificially synthesized sequence
DLVMTQSPSSLSASVGDRVTITCKSSQSLLDSDDGNTYLAWYQQKPGKAPKLLIYWLSYRASGVPSRFSGSGSGTDFTLTISSLQPEDFATYYCQQYYYHMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 46 - protein, 448 aa, Artificial Sequence
  an artificially synthesized sequence
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 299 aa, Homo sapiens
MDYKDDDDKTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 48 - protein, 312 aa, Homo sapiens
MHHHHHHAKDFQDIQQLSSEENDHPFHQGEGPGTRRLNPRRGNPFLKGPPPAQPLAQRLCSMVCFSLLALSFNILLLVVICVTGSQSAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 49 - protein, 402 aa, Artificial Sequence
  eGFP-NanoLuc Lgbit
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDISGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 50 - protein, 433 aa, Artificial Sequence
  AKAP1-eGFP, NanoLuc Lgbit
MAIQFRSLFPLALPGMLALLGWWWFFSRKKSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDISGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 51 - protein, 473 aa, Artificial Sequence
  3D8H-SG1.S3n.SmBiT
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSPGKGGGSGGGSGGGSVTGYRLFEEIL

SEQ ID NO: 52 - protein, 220 aa, Artificial Sequence; identical to SEQ ID NO: 32
  hT4VL-SK1

SEQ ID NO: 53 - protein, 473 aa, Artificial Sequence
  2C10VH-SG1.S3n.SmBiT
QVQLQQPGAELVKPGASVKLSCKASGYTFTRFWINWVRQRPGQGLEWIGNIYPGSSSINYNEKFKNKATLTVDTSSSTAYMQLSSLTSDDSAVYYCARRRYRSSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSPGKGGGSGGGSGGGSVTGYRLFEEIL

SEQ ID NO: 54 - protein, 214 aa, Artificial Sequence; identical to SEQ ID NO: 35
  2C10VL-SK1

SEQ ID NO: 55 - protein, 472 aa, Artificial Sequence
  AGA0078Ha-SG1.S3p.SmBiT
QSVEESGGRLVTPGTPLTLTCTVSGFSLSNYAMSWVRQAPGKGLQYIGIIYTGGSAYYASWAKGRFTISRTSTTVDLKMTSLTTEDTATYFCARDFASSSGYYGYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSPGKGGGSGGGSGGGSVTGYRLFEEIL

SEQ ID NO: 56 - protein, 215 aa, Artificial Sequence
  AGA0078La-k0MC
AVVLTQTASPVSAPVGGTVTIKCQASESIGNALAWYQQKPGQPPKLLIYLGSTLASGVPSRFSGSGSGTEFTLTISDLECADVGTYYCQKIVGTSSMTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 57 - protein, 215 aa, Artificial Sequence
  AGA0078L0030-k0MC
AVVLTQTASPVSAPVGGTVTIKCQASESIDNALAWYQQKPGQPPKLLIYLGSTLASGVPSRFSGSGSGTEFTLTISDLECADVGTYYCQKIHGTSSMTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 58 - protein, 469 aa, Artificial Sequence
  IC17HdK-SG1.S3n.SmBiT
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSPGKGGGSGGGSGGGSVTGYRLFEEIL

SEQ ID NO: 59 - protein, 214 aa, Artificial Sequence; identical to SEQ ID NO: 41
  IC17L-SK1

SEQ ID NO: 60 - protein, 469 aa, Artificial Sequence
  IC17HdK-SG1.S3p.SmBiT
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSPGKGGGSGGGSGGGSVTGYRLFEEIL

SEQ ID NO: 61 - protein, 30 aa, Homo sapiens
MAIQFRSLFPLALPGMLALLGWWWFFSRKK

SEQ ID NO: 62 - protein, 158 aa, Artificial Sequence
  Nanoluc LgBit
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 63 - protein, 11 aa, Artificial Sequence
  Nanoluc SmBit
VTGYRLFEEIL

SEQ ID NO: 64 - protein, 171 aa, Artificial Sequence
  Nanoluc
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 65 - protein, 239 aa, Artificial Sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 66 - protein, 464 aa, Artificial Sequence
  PF1H-G4T1K439E.Avi
QVQLQESGPGLVKPSETLSLTCAVSGYSISDDHAWSWVRQPPGEGLEWIGYISYSGITNYNPSLKGRVTISRDTSKNQFSLKLSSVTAADTAAYYCARVLARITAMDYWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSPGGGGSGLNDIFEAQKIEWHE

SEQ ID NO: 67 - protein, 214 aa, Artificial Sequence
  PF1L-KT0
DIQMTQSPSSLSASVGDSVTITCQASQDISSYLNWYQQKPGKAPELLIYYGSELHSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 464 aa, Artificial Sequence
  AGA0008Ha-G4T1K439E.Avi
QSVEESGGRLVTPGTPLTLTCTVSGFSLSSYAMGWVRQAPGKGLEYIGFINTGGSPYYASWAKGRFTISRTSTTVDLKMTSPTTEDTATYFCARDYDTSSGYYGYFNLWGPGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSPGGGGSGLNDIFEAQKIEWHE

SEQ ID NO: 69 - protein, 216 aa, Artificial Sequence
  AGA0008La-k0MC
AQVLTQTPASVSAAVGGTVTIKCQASENIDSYLAWYQQKPGQPPKLLIYRASTLASGVPSRFKGSGSGTPFTLTISDLECADAATYYCQRYVSLNSGDAFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 70 - protein, 445 aa, Artificial Sequence
  3D8VH-G4T1E356K
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSP

SEQ ID NO: 71 - protein, 240 aa, Artificial Sequence
  hT4VL-KT0.Avi
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYYYHMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 72 - protein, 240 aa, Artificial Sequence; identical to SEQ ID NO: 45
  hT4VL03-KT0.Avi

SEQ ID NO: 73 - protein, 445 aa, Artificial Sequence
  2C10VH-G4T1E356K
QVQLQQPGAELVKPGASVKLSCKASGYTFTRFWINWVRQRPGQGLEWIGNIYPGSSSINYNEKFKNKATLTVDTSSSTAYMQLSSLTSDDSAVYYCARRRYRSSYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSP

SEQ ID NO: 74 - protein, 234 aa, Artificial Sequence
  2C10VL-KT0.Avi
ETTVTQSPASLSVATGEKVTIRCITNTDIDDDMNWYQQKPGEPPKLLISEGNTLRPGVPSRFSSSGYGTDFVFTIENTLSEDVADYCCLQSDNMPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 75 - protein, 441 aa, Artificial Sequence
  m3E10VH.D31N-G4T1E356K
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSP

SEQ ID NO: 76 - protein, 238 aa, Artificial Sequence
  m3E10VL-KT0.Avi
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 77 - protein, 444 aa, Artificial Sequence
  MRAH-G4T1K439E
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 78 - protein, 234 aa, Artificial Sequence
  MRAL-KT0.Avi
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 79 - protein, 444 aa, Artificial Sequence
  H237-G4T1K439E
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 80 - protein, 234 aa, Artificial Sequence
  L104-KT0.Avi
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 81 - protein, 444 aa, Artificial Sequence
  AGA0008Ha-G4T1K439E
QSVEESGGRLVTPGTPLTLTCTVSGFSLSSYAMGWVRQAPGKGLEYIGFINTGGSPYYASWAKGRFTISRTSTTVDLKMTSPTTEDTATYFCARDYDTSSGYYGYFNLWGPGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 82 - protein, 236 aa, Artificial Sequence
  AGA0008La-KT0.Avi
AQVLTQTPASVSAAVGGTVTIKCQASENIDSYLAWYQQKPGQPPKLLIYRASTLASGVPSRFKGSGSGTPFTLTISDLECADAATYYCQRYVSLNSGDAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 83 - protein, 447 aa, Artificial Sequence
  A0841H0217-G4T1K439E
EVQLVQSGAEVKKPGESLKISCKGSGYSFDSYAMGWIRQPPGKGLEWIGFINTGGSHYYASWAKGQVTISADKSISTAYLQWSSLKASDTAMYYCARDYHTSSGYYGYFNLWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 84 - protein, 236 aa, Artificial Sequence
  A0811L0125-KT0.Avi
EIVLTQSPATLSLSPGERATLSCQASENIHSYLAWYQQKPGQPPKLLIYRASTLASGIPARFSGSGSGTDFTLTISSLEPEDFAVYYCQRYVHLNEGDAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 85 - protein, 441 aa, Artificial Sequence
  IC17HdK-G4T1E356K
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSP

SEQ ID NO: 86 - protein, 234 aa, Artificial Sequence
  IC17L-KT0.Avi
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 87 - protein, 441 aa, Artificial Sequence
  IC17HdK-G4T1K439E
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 88 - protein, 444 aa, Artificial Sequence
  H54-G4T1K439E
QVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFRRGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSP

SEQ ID NO: 89 - protein, 234 aa, Artificial Sequence
  L28-KT0.Avi
DIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHE

SEQ ID NO: 90 - protein, 17 aa, Artificial Sequence
  Avitag
GGLNDIFEAQKIEWHED

(19) United States
(12) Patent Application Publication
(10) Pub. No.: US 2021/0292409 A1
(43) Pub. Date: Sep. 23, 2021

(54) TARGET CELL SPECIFIC CYTOSOL-PENETRATING ANTIGEN-BINDING MOLECULES

(71) Applicant: Chugai Seiyaku Kabushiki Kaisha, Tokyo (JP)

(72) Inventors: Yuri IKAWA, Singapore (SG); Tomoyuki IGAWA, Singapore (SG); Nasa SAVORY, Shizuoka (JP); Kazuki KATO, Shizuoka (JP); Takashi SUZUKI, Shizuoka (JP); Shun SHIMIZU, Shizuoka (JP); Shinya ISHIII, Singapore (SG); Koichiro SAKA, Shizuoka (JP); Wei Shiong Adrian HO, Singapore (SG)

(21) Appl. No.: 17/262,105

(22) PCT Filed: Jul. 22, 2019
(86) PCT No.: PCT/JP2019/028649

§ 371 Date: Jan. 21, 2021

(30) Foreign Application Priority Data
Jul. 23, 2018 (JP) 2018-137464

(51) Int. Cl. C07K 16/28

(52) U.S. Cl. CPC C07K 16/28; A61K 38/00; C07K 2319/30

(57) ABSTRACT

The invention relates to cytosol penetrating antigen binding molecules containing cell surface antigen binding domains, cytosolic antigen binding domains and cytosol penetrating domains; pharmaceutical compositions comprising said antigen binding molecules; methods of delivering said antigen binding molecules specifically into cytosols of target cells; methods of depleting, suppressing or activating cytosolic antigens specifically in target cells by using said antigen binding molecules; and pharmaceutical compositions for preventing or treating diseases in patients comprising said antigen binding molecules. The invention also relates to cytosol penetrating antigen binding molecules containing cytosol penetrating domains and Fc regions, wherein the Fc regions comprising one or more amino acid modifications that enhance multimerization of said cytosol penetrating antigen binding molecules. The invention also relates to an antigen-binding molecule comprising a cell surface antigen-binding domain, a cytosol-penetrating domain, which is conjugated to a heterologous moiety. Further, the invention relates to a method for detecting a cytosol-penetrating ability of a molecule comprising split fluorescent proteins.

### TECHNICAL FIELD

The present disclosure relates to antigen-binding molecules comprising a cell surface antigen-binding domain, a cytosolic antigen-binding domain, and a cytosol-penetrating domain; pharmaceutical compositions comprising the antigen-binding molecule; methods for delivering the antigen-binding molecule specifically into the cytosol of a target cell; methods of removing, suppressing, or activating a cytosolic antigen in a target cell-specific manner by using the antigen-binding molecule; and pharmaceutical compositions for diagnosing, preventing, or treating a disease in a subject comprising the antigen-binding molecule. The present disclosure also relates to antigen-binding molecules comprising a cytosol-penetrating domain and an Fc region, the Fc region comprising one or more amino acid alterations for enhancing formation of a multimer of the antigen-binding molecule.

### BACKGROUND ART

Antibodies are proteins which specifically bind to an antigen with high affinity. It is known that various molecules ranging from low-molecular weight compounds to proteins can be antigens. Since the technique for producing monoclonal antibodies was developed, antibody modification techniques have advanced, making it easy to obtain antibodies that recognize a particular molecule.

Antibodies are drawing attention as pharmaceuticals because they are highly stable in blood plasma and have less side effects. Not only do antibodies bind to an antigen and exhibit agonistic or antagonistic effects, but they also induce cytotoxic activity mediated by effector cells (also referred to as effector functions) including ADCC (Antibody Dependent Cytotoxicity), ADCP (Antibody Dependent Cell Phagocytosis), and CDC (Complement Dependent Cytotoxicity). Taking advantage of these antibody functions, pharmaceuticals for cancer, immune diseases, chronic diseases, infections, etc. have been developed (Nat Rev Drug Discov. 2018 March; 17(3):197-223. (NPL 1)).

Meanwhile, antigens targeted by antibody pharmaceuticals are limited to antigens on cell membrane or antigens outside cells, as full-length IgG molecules are of high molecular weight (about 150 kDa) and generally do not exhibit cell permeability. Thus, intrabodies (antibodies or antibody fragments expressed inside cells), antibody-CPP complexes produced by fusion with a cell penetrating peptide (CPP), the protein transfection method, etc. were developed and reported as technologies to allow antibodies to act on intracellular antigens (MAbs. 2011 January-February; 3(1):3-16. (NPL 2)).

Some naturally occurring full length antibodies are known to have cell-penetrating ability. For example, anti-DNA autoantibodies identified from systemic lupus erythematosus (SLE) patients and MRL-mpj/lpr lupus model mice were reported to show cell-penetrating ability (Sci Rep. 2015 Jul. 9; 5:12022. (NPL 3), Mol Immunol. 2015 October; 67(2 Pt B):377-87. (NPL 4)). Some of the anti-DNA antibodies also show DNA hydrolysis activity or DNA repair-inhibiting activity and exhibit cytotoxicity, in addition to the cell-penetrating ability (WO 2012135831 (PTL 1), Sci Rep. 2014 Aug. 5; 4:5958 (NPL 5)). There is also a report on an antibody prepared by humanizing an antibody isolated from an SLE model mouse that has ability to escape from endosomes into the cytosol, and combining it with a heavy chain variable region that can bind to activated Ras (WO2016013871A1 (PTL 2)).

### CITATION LIST

### Patent Literature

[PTL 1] WO2012/135831
[PTL 2] WO2016/013871A1

### Non-Patent Literature

[NPL 1] Nat Rev Drug Discov. 2018 March; 17(3):197-223.
[NPL 2] MAbs. 2011 January-February; 3(1):3-16.
[NPL 3] Sci Rep. 2015 Jul. 9; 5:12022.
[NPL 4] Mol Immunol. 2015 October; 67(2 Pt B):377-87.
[NPL 5] Sci Rep. 2014 August 5; 4:5958

### SUMMARY OF INVENTION

### Technical Problem

In one non-limiting embodiment, an objective of the present invention is to provide an antigen-binding molecule that is to be delivered specifically to the cytosol of a target cell, a pharmaceutical composition comprising the antigen-binding molecule, a method of using the antigen-binding molecule, a method of producing the antigen-binding molecule, and a method for delivering an antigen-binding molecule specifically into the cytosol of a target cell.

### Solution to Problem

In one non-limiting embodiment, the present inventors found that multifunctional antigen-binding molecules comprising a cytosol-penetrating antibody or a fragment of the antibody, an antibody binding to a cell surface antigen or a fragment of the antibody, and/or an antibody binding to a cytosolic antigen or a fragment of the antibody in combination are delivered into cytosol in a target cell-specific manner. In one non-limiting embodiment, it is expected that these antigen-binding molecules bind to a cell surface antigen, and thereby undergo internalization in a target cell-specific manner and are transferred into cytosol. In one non-limiting embodiment, the present inventors found that the cytosol-penetrating ability of the multifunctional antigen-binding molecules is potentiated via the multimerization of the multifunctional antigen-binding molecules.

The present disclosure is based on these findings, and specifically encompasses the embodiments described below for example.
[0] An antigen-binding molecule comprising a cell surface antigen-binding domain, a cytosolic antigen-binding domain, and a cytosol-penetrating domain.
   [0A] The antigen-binding molecule of [0], which does not bind to an antigen expressed on the cell surface that is different from aforesaid cell surface antigen.
   [0B] The antigen-binding molecule of [0], wherein the cytosolic antigen-binding domain and the cytosol-penetrating domain do not bind to an antigen expressed on the cell surface that is different from aforesaid cell surface antigen.
   [0C] The antigen-binding molecule of [0] or [0B], wherein the cytosolic antigen-binding domain and the cytosol-penetrating domain do not bind to an antigen expressed on the cell surface.
   [0D] The antigen-binding molecule of any one of [0] to [0C], wherein the antigen-binding molecule is a multifunctional antibody or a multifunctional antibody derivative.
[1] An antigen-binding molecule comprising a first and a second Fab regions, wherein
   (a) the first Fab region binds specifically to a cell surface antigen, and
   (b) the second Fab region comprises (i) a pair of a heavy chain variable region (VH) binding specifically to a cytosolic antigen and a light chain variable region (VL) having cytosol-penetrating ability or (ii) a pair of a heavy chain variable region (VH) having cytosol-penetrating ability and a light chain variable region (VL) binding specifically to a cytosolic antigen.
[1A] An antigen-binding molecule comprising a first and a second Fab regions, wherein
   (a) the first Fab region comprises a pair of a heavy chain variable region (VH) binding specifically to a cell surface antigen and a light chain variable region (VL) having cytosol-penetrating ability, and
   (b) the second Fab region comprises a pair of a heavy chain variable region (VH) binding specifically to a cytosolic antigen and a light chain variable region (VL) having cytosol-penetrating ability.
[1B] An antigen-binding molecule comprising a first and a second Fab regions, wherein
   (a) the first Fab region comprises a pair of a heavy chain variable region (VH) having cytosol-penetrating ability and a light chain variable region (VL) binding specifically to a cell surface antigen, and
   (b) the second Fab region comprises a pair of a heavy chain variable region (VH) having cytosol-penetrating ability and a light chain variable region (VL) binding specifically to a cytosolic antigen.
[1C] The antigen-binding molecule of any one of [1]-[1B], further comprising an Fc region.
[1D] The antigen-binding molecule of any one of [1]-[1C], wherein the Fc region comprises a modification that enhances association of a first Fc subunit and a second Fc subunit.
[2] An antigen-binding molecule comprising a first and a second Fab regions and a single-chain unit, wherein
   (a) the first Fab region binds specifically to a cell surface antigen,
   (b) the second Fab region has cytosol-penetrating ability, and
   (c) the single-chain unit binds specifically to a cytosolic antigen.
[2A] The antigen-binding molecule of [2], wherein the single-chain unit is fused to
   (i) the N terminus of a heavy chain variable region (VH) in the first Fab region and/or the N terminus of a heavy chain variable region (VH) of the second Fab region,
   (ii) the N terminus of a light chain variable region (VL) of the first Fab region and/or the N terminus of a light chain variable region (VL) of the second Fab region, or
   (iii) the C terminus of a light chain constant region (CL) of the first Fab region and/or the C terminus of a light chain constant region (CL) of the second Fab region.
[2B] The antigen-binding molecule of [2], wherein the antigen-binding molecule further comprises an Fc region comprising a first Fc subunit and a second Fc subunit, and wherein the single-chain unit is fused to
   (i) the C terminus of the first Fc subunit, or
   (ii) the C terminus of the second Fc subunit.
[2C] The antigen-binding molecule of [2B], wherein the first Fc subunit is heavy chain constant region CH2 and CH3 domains of an IgG antibody, and wherein the second Fc subunit is heavy chain constant region CH2 and CH3 domains of an IgG antibody.
[2D] The antigen-binding molecule of [2B] or [2C], wherein the first Fc subunit and the second Fc subunit comprise a modification that enhances association of the first Fc subunit and the second Fc subunit.
[2E] The antigen-binding molecule of any one of [2]-[2D], wherein the single-chain unit is a single-domain antibody variable region or a single-chain antibody (scFv).
[2F] The antigen-binding molecule of [2E], wherein the single-domain antibody variable region is a heavy-chain antibody variable region (Variable region of Heavy chain of Heavy chain antibody: VHH), a heavy chain variable region (VH), a light chain variable region (VL), or a variable region of an immunoglobulin new antigen receptor (VNAR).
[3] An antigen-binding molecule comprising a first and a second Fab regions and a single-chain unit, wherein
   (a) the first Fab region binds specifically to a cytosolic antigen,
   (b) the second Fab region has cytosol-penetrating ability, and
   (c) the single-chain unit binds specifically to a cell surface antigen.
[3A] The antigen-binding molecule of [3], wherein the single-chain unit is fused to
   (i) the N terminus of a heavy chain variable region (VH) in the first Fab region and/or the N terminus of a heavy chain variable region (VH) of the second Fab region,
   (ii) the N terminus of a light chain variable region (VL) of the first Fab region and/or the N terminus of a light chain variable region (VL) of the second Fab region, or
   (iii) the C terminus of a light chain constant region (CL) of the first Fab region and/or the C terminus of a light chain constant region (CL) of the second Fab region.
[3B] The antigen-binding molecule of [3], wherein the antigen-binding molecule further comprises an Fc region comprising a first Fc subunit and a second Fc subunit, and wherein the single-chain unit is fused to
   (i) the C terminus of the first Fc subunit, or
   (ii) the C terminus of the second Fc subunit.
[3C] The antigen-binding molecule of [3B], wherein the first Fc subunit is heavy chain constant region CH2 and CH3 domains of an IgG antibody, and wherein the second Fc subunit is heavy chain constant region CH2 and CH3 domains of an IgG antibody.
[3D] The antigen-binding molecule of [3B] or [3C], wherein the first Fc subunit and the second Fc subunit comprise a modification that enhances association of the first Fc subunit and the second Fc subunit.
[3E] The antigen-binding molecule of any one of [3]-[3D], wherein the single-chain unit is a single-domain antibody variable region or a single-chain antibody (scFv).
[3F] The antigen-binding molecule of [3E], wherein the single-domain antibody variable region is a heavy-chain antibody variable region (VHH), a heavy chain variable region (VH), a light chain variable region (VL), or a variable region of an immunoglobulin new antigen receptor (VNAR).
[3G] An antigen-binding molecule comprising a first and a second Fab regions and a single-chain unit, wherein
   (a) the first Fab region binds specifically to a cell surface antigen,
   (b) the second Fab region binds specifically to a cytosolic antigen, and
   (c) the single-chain unit has cytosol-penetrating ability.
[3H] The antigen-binding molecule of [3G], wherein the single-chain unit is fused to
   (i) the N terminus of a heavy chain variable region (VH) in the first Fab region and/or the N terminus of a heavy chain variable region (VH) of the second Fab region,
   (ii) the N terminus of a light chain variable region (VL) of the first Fab region and/or the N terminus of a light chain variable region (VL) of the second Fab region, or
   (iii) the C terminus of a light chain constant region (CL) of the first Fab region and/or the C terminus of a light chain constant region (CL) of the second Fab region.
[3I] The antigen-binding molecule of [3G, wherein the antigen-binding molecule further comprises an Fc region comprising a first Fc subunit and a second Fc subunit, and wherein the single-chain unit is fused to
   (i) the C terminus of the first Fc subunit, or
   (ii) the C terminus of the second Fc subunit.
[3J] The antigen-binding molecule of [3I], wherein the first Fc subunit is heavy chain constant region CH2 and CH3 domains of an IgG antibody, and wherein the second Fc subunit is heavy chain constant region CH2 and CH3 domains of an IgG antibody.
[3K] The antigen-binding molecule of [3I] or [3J], wherein the first Fc subunit and the second Fc subunit comprise a modification that enhances association of the first Fc subunit and the second Fc subunit.
[3L] The antigen-binding molecule of any one of [3G]-[3K], wherein the single-chain unit is a single-domain antibody variable region or a single-chain antibody (scFv).
[3M] The antigen-binding molecule of [3L], wherein the single-domain antibody variable region is a heavy-chain antibody variable region (VHH), a heavy chain variable region (VH), a light chain variable region (VL), or a variable region of an immunoglobulin new antigen receptor (VNAR).
[4] An antigen-binding molecule comprising a first and a second Fab regions and a single-chain unit, wherein
   (a) the first and the second Fab regions comprise (i) a pair of a heavy chain variable region (VH) binding specifically to a cell surface antigen and a light chain variable region (VL) having cytosol-penetrating ability or (ii) a pair of a heavy chain variable region (VH) having cytosol-penetrating ability and a light chain variable region (VL) binding specifically to a cell surface antigen, and
   (b) the single-chain unit binds specifically to a cytosolic antigen.
[4A] The antigen-binding molecule of [4], wherein the single-chain unit is fused to
   (i) the N terminus of a heavy chain variable region (VH) in the first Fab region and/or the N terminus of a heavy chain variable region (VH) of the second Fab region,
   (ii) the N terminus of a light chain variable region (VL) of the first Fab region and/or the N terminus of a light chain variable region (VL) of the second Fab region, or
   (iii) the C terminus of a light chain constant region (CL) of the first Fab region and/or the C terminus of a light chain constant region (CL) of the second Fab region.
[4B] The antigen-binding molecule of [4], wherein the antigen-binding molecule further comprises an Fc region comprising a first Fc subunit and a second Fc subunit, and wherein the single-chain unit is fused to
   (i) the C terminus of the first Fc subunit, or
   (ii) the C terminus of the second Fc subunit.
[4C] The antigen-binding molecule of [4B], wherein the first Fc subunit is heavy chain constant region CH2 and CH3 domains of an IgG antibody, and wherein the second Fc subunit is heavy chain constant region CH2 and CH3 domains of an IgG antibody.
[4D] The antigen-binding molecule of [4B] or [4C], wherein the first Fc subunit and the second Fc subunit comprise a modification that enhances association of the first Fc subunit and the second Fc subunit.
[4E] The antigen-binding molecule of any one of [4]-[4D], wherein the single-chain unit is a single-domain antibody variable region or a single-chain antibody (scFv).
[4F] The antigen-binding molecule of [4E], wherein the single-domain antibody variable region is a heavy-chain antibody variable region (VHH), a heavy chain variable region (VH), a light chain variable region (VL), or a variable region of an immunoglobulin new antigen receptor (VNAR).
[4G] An antigen-binding molecule comprising a first and a second single-domain antibody variable regions and a single-chain unit, wherein
   (a) the first single-domain antibody variable region binds specifically to a cell surface antigen,
   (b) the second single-domain antibody variable region has cytosol-penetrating ability, and
   (c) the single-chain unit binds specifically to a cytosolic antigen.
[4H] The antigen-biding molecule of [4G], wherein the single-chain unit is fused to the N terminus of the first single-domain antibody variable region and/or the N terminus of the second single-domain antibody variable region.
[4I] The antigen-binding molecule of [4G], wherein the antigen-binding molecule further comprises an Fc region comprising a first Fc subunit and a second Fc subunit, and wherein the single-chain unit is fused to
   (i) the C terminus of the first Fc subunit, or
   (ii) the C terminus of the second Fc subunit.
[4J] The antigen-binding molecule of [4I], wherein the first Fc subunit is heavy chain constant region CH2 and CH3 domains of an IgG antibody, and wherein the second Fc subunit is heavy chain constant region CH2 and CH3 domains of an IgG antibody.
[4K] The antigen-binding molecule of [4] or [4J], wherein the first Fc subunit and the second Fc subunit comprise a modification that enhances association of the first Fc subunit and the second Fc subunit.
[4L] The antigen-binding molecule of any one of [4G]-[4K], wherein the single-chain unit is a single-domain antibody variable region or a single-chain antibody (scFv).
[4M] The antigen-binding molecule of any one of [4G]-[4L], wherein the single-domain antibody variable region is a heavy-chain antibody variable region (VHH), a heavy chain variable region (VH), a light chain variable region (VL), or a variable region of an immunoglobulin new antigen receptor (VNAR).
[5] An antigen-binding molecule comprising a first and a second polypeptide chains, wherein
   (a) the first polypeptide chain comprises a first VD1-(X1)n-VD2-C-(X2)n, in which
      VD1 is a first heavy chain variable region binding specifically to a cytosolic antigen,
      VD2 is a second heavy chain variable region binding specifically to a cell surface antigen,
      C is a heavy chain constant region CH1,
      X1 is a linker other than CH1,
      X2 is an Fc region, and
      n is 0 or 1,
   (b) the second polypeptide chain comprises a second VD1-(X1)n-VD2-C-(X2)n, in which
      VD1 is a first light chain variable region having cytosol-penetrating ability,
      VD2 is a second light chain variable region binding specifically to a cell surface antigen,
      C is a light chain constant region CL,
      X1 is a linker other than CL,
      X2 does not comprise an Fc region, and
      n is 0 or 1.
[5A] An antigen-binding molecule comprising a first and a second polypeptide chains, wherein
   (a) the first polypeptide chain comprises a first VD1-(X1)n-VD2-C-(X2)n, in which
      VD1 is a first heavy chain variable region having cytosol-penetrating ability,
      VD2 is a second heavy chain variable region binding specifically to a cell surface antigen,
      C is a heavy chain constant region CH1,
      X1 is a linker other than CH1,
      X2 is an Fc region, and
      n is 0 or 1,
   (b) the second polypeptide chain comprises a second VD1-(X1)n-VD2-C-(X2)n, in which
      VD1 is a first light chain variable region binding specifically to a cytosolic antigen,
      VD2 is a second light chain variable region binding specifically to a cell surface antigen,
      C is a light chain constant region CL,
      X1 is a linker other than CL,
      X2 does not comprise an Fc region, and
      n is 0 or 1.
[5B] An antigen-binding molecule comprising a first and a second polypeptide chains, wherein
   (a) the first polypeptide chain comprises a first VD1-(X1)n-VD2-C-(X2)n, in which
      VD1 is a first heavy chain variable region binding specifically to a cell surface antigen,
      VD2 is a second heavy chain variable region binding specifically to a cytosolic antigen,
      C is a heavy chain constant region CH1,
      X1 is a linker other than CH1,
      X2 is an Fc region, and
      n is 0 or 1,
   (b) the second polypeptide chain comprises a second VD1-(X1)n-VD2-C-(X2)n, in which
      VD1 is a first light chain variable region binding specifically to a cell surface antigen,
      VD2 is a second light chain variable region having cytosol-penetrating ability,
      C is a light chain constant region CL,
      X1 is a linker other than CL,
      X2 does not comprise an Fc region, and
      n is 0 or 1.
[5C] An antigen-binding molecule comprising a first and a second polypeptide chains, wherein
   (a) the first polypeptide chain comprises a first VD1-(X1)n-VD2-C-(X2)n, in which
      VD1 is a first heavy chain variable region binding specifically to a cell surface antigen,
      VD2 is a second heavy chain variable region having cytosol-penetrating ability,
      C is a heavy chain constant region CH1,
      X1 is a linker other than CH1,
      X2 is an Fc region, and
      n is 0 or 1,
   (b) the second polypeptide chain comprises a second VD1-(X1)n-VD2-C-(X2)n, in which
      VD1 is a first light chain variable region binding specifically to a cell surface antigen,
      VD2 is a second light chain variable region binding specifically to a cytosolic antigen,
      C is a light chain constant region CL,
      X1 is a linker other than CL,
      X2 does not comprise an Fc region, and
      n is 0 or 1.
[6] An antigen-binding molecule comprising a first, a second, and a third Fab regions and an Fc region, wherein
   (a) the first Fab region binds specifically to a cell surface antigen,
   (b) the second and the third Fab regions comprise (i) a pair of a heavy chain variable region binding specifically to a cytosolic antigen and a light chain variable region having cytosol-penetrating ability or (ii) a pair of a heavy chain variable region having cytosol-penetrating ability and a light chain variable region binding specifically to a cytosolic antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.
[6A] The antigen-binding molecule of [6], wherein the first Fc subunit is heavy chain constant region CH2 and CH3 domains of an IgG antibody, and wherein the second Fc subunit is heavy chain constant region CH2 and CH3 domains of an IgG antibody.
[6B] The antigen-binding molecule of [6], wherein the first Fab region and/or the second Fab region comprises any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[6C] An antigen-binding molecule comprising a first, a second, and a third Fab regions and an Fc region, wherein
   (a) the first and the third Fab regions comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen,
   (b) the second Fab region binds specifically to a cell surface antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.
[6D] The antigen-binding molecule of [6C], wherein the first Fc subunit is heavy chain constant region CH2 and CH3 domains of an IgG antibody, and wherein the second Fc subunit is heavy chain constant region CH2 and CH3 domains of an IgG antibody.
[6E] The antigen-binding molecule of [6C], wherein the first Fab region and/or the second Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[6F] An antigen-binding molecule comprising a first, a second, and a third Fab regions and an Fc region, wherein
   (a) the first Fab region and the second Fab region comprise (i) a pair of a heavy chain variable region binding specifically to a cytosolic antigen and a light chain variable region having cytosol-penetrating ability or (ii) a pair of a heavy chain variable region having cytosol-penetrating ability and a light chain variable region binding specifically to a cytosolic antigen,
   (b) the third Fab region binds specifically to a cell surface antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.
[6G] The antigen-binding molecule of [6F], wherein the first Fc subunit is heavy chain constant region CH2 and CH3 domains of an IgG antibody, and wherein the second Fc subunit is heavy chain constant region CH2 and CH3 domains of an IgG antibody.
[6H] The antigen-binding molecule of [6F], wherein the first Fab region and/or the second Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[6I] The antigen-binding molecule of any one of [6]-[6H], wherein the first Fc subunit and the second Fc subunit comprise a modification that enhances association of the first Fc subunit and the second Fc subunit.
[7] An antigen-binding molecule comprising a first, a second, a third, and a fourth Fab regions and an Fc region, wherein
   (a) one Fab region selected from the first, the second, the third, and the fourth Fab regions binds specifically to a cell-surface antigen,
   (b) three Fab regions other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.
[7A] The antigen-binding molecule of [7], wherein the first Fc subunit is heavy chain constant region CH2 and CH3 domains of an IgG antibody, and wherein the second Fc subunit is heavy chain constant region CH2 and CH3 domains of an IgG antibody.
[7B] The antigen-binding molecule of [7] or [7A], wherein the first Fab region and/or the second Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[7C] The antigen-binding molecule of [7] or [7A], wherein the third Fab region and/or the fourth Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
      and wherein the third Fab region and the fourth Fab region do not comprise the same exchange.
[7D] An antigen-binding molecule comprising a first, a second, a third, and a fourth Fab regions and an Fc region, wherein
   (a) two Fab regions selected from the first, the second, the third, and the fourth Fab regions bind specifically to a cell surface antigen,
   (b) two Fab regions other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.
[7E] The antigen-binding molecule of [7D], wherein the first Fc subunit is heavy chain constant region CH2 and CH3 domains of an IgG antibody, and wherein the second Fc subunit is heavy chain constant region CH2 and CH3 domains of an IgG antibody.
[7F] The antigen-binding molecule of [7D] or [7E], wherein the first Fab region and/or the second Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[7G] The antigen-binding molecule of [7D] or [7E], wherein the third Fab region and/or the fourth Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the third Fab region and the fourth Fab region do not comprise the same exchange.
[7H] An antigen-binding molecule comprising a first, a second, a third, and a fourth Fab regions and an Fc region, wherein
   (a) three Fab regions selected from the first, the second, the third, and the fourth Fab regions bind specifically to a cell surface antigen,
   (b) one Fab region other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen,
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.
[7I] The antigen-binding molecule of [7H], wherein the first Fc subunit is heavy chain constant region CH2 and CH3 domains of an IgG antibody, and wherein the second Fc subunit is heavy chain constant region CH2 and CH3 domains of an IgG antibody.
[7J] The antigen-binding molecule of [7H] or [7I], wherein the first Fab region and/or the second Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
   and wherein the first Fab region and the second Fab region do not comprise the same exchange.
[7K] The antigen-binding molecule of [7H] or [7I], wherein the third Fab region and/or the fourth Fab region comprise any one exchange selected from
   (i) an exchange between a Fab light chain variable region (VL) and a Fab heavy chain variable region (VH),
   (ii) an exchange between a Fab light chain constant region (CL) and a Fab heavy chain constant region (CH1), and
   (iii) an exchange between a Fab light chain (VL-CL) and a Fab heavy chain (VH-CH1),
      and wherein the third Fab region and the fourth Fab region do not comprise the same exchange.
[7L] The antigen-binding molecule of any one of [7]-[7K], wherein the first Fc subunit and the second Fc subunit comprise a modification that enhances association of the first Fc subunit and the second Fc subunit.
[8] An antigen-binding molecule comprising a region having cytosol-penetrating ability and an Fc region, wherein the Fc region comprises one or more amino acid alterations for enhancing formation of a multimer of the antigen-binding molecule, and wherein the antigen-binding molecule has an elevated cytosol-penetrating ability as compared to an antigen-binding molecule comprising a parent Fc region which does not comprise the one or more amino acid alterations.
[8A] The antigen-binding molecule of [8], wherein the multimer is a dimer, a trimer, a tetramer, a pentamer, or a hexamer.
[8B] The antigen-binding molecule of [8], wherein the multimer is a hexamer.
[8C] The antigen-binding molecule of any one of [8]-[8B], wherein the one or more amino acid alterations are one or more amino acid alterations at positions selected from the group consisting of EU345, EU430, EU440, EU437, and EU248, wherein the numbers represent positions of substitutions according to EU numbering.
[8D] The antigen-binding molecule of any one of [8]-[8C], wherein the one or more amino acid alterations are the combination E345R/E430G/S440Y or the combination T437R/K248E, wherein the numbers represent positions of substitutions according to EU numbering.
[8E] The antigen-binding molecule of any one of [8]-[8D], further comprising a cell surface antigen-binding domain and a cytosolic antigen-binding domain.
[8F] The antigen-binding molecule of any one of [8]-[8E], wherein the Fc region further comprises a modification that enhances association of a first Fc subunit and a second Fc subunit.
[9] An antigen-binding molecule comprising a first and a second Fab regions and an Fc region, wherein
   (a) the first Fab region binds specifically to a cell surface antigen,
   (b) the second Fab region comprises (i) a pair of a heavy chain variable region (VH) binding specifically to a cytosolic antigen and a light chain variable region (VL) having cytosol-penetrating ability, or (ii) a pair of a heavy chain variable region (VH) having cytosol-penetrating ability and a light chain variable region (VL) binding specifically to a cytosolic antigen, and
   (c) the Fc region comprises one or more amino acid alterations for enhancing formation of a multimer of the antigen-binding molecule.
[9A] A complex of antigen-binding molecules which is a multimer comprising two or more of an antigen-binding molecule, wherein the antigen-binding molecule comprises a first and a second Fab regions and an Fc region, wherein
   (a) the first Fab region comprises a pair of a heavy chain variable region (VH) and a light chain variable region (VL) which bind specifically to a cell surface antigen,
   (b) the second Fab region comprises (i) a pair of a heavy chain variable region (VH) binding specifically to a cytosolic antigen and a light chain variable region (VL) having cytosol-penetrating ability, or (ii) a pair of a heavy chain variable region (VH) having cytosol-penetrating ability and a light chain variable region (VL) binding specifically to a cytosolic antigen, and
   (c) the Fc region comprises one or more amino acid alterations for enhancing formation of the multimer of the antigen-binding molecule.
[9B] The antigen-binding molecule or the complex thereof of [9] or [9A], wherein the multimer is a dimer, a trimer, a tetramer, a pentamer, or a hexamer.
[9C] The antigen-binding molecule or the complex thereof of [9] or [9A], wherein the multimer is a hexamer.
[9D] The antigen-binding molecule or the complex thereof of any one of [9]-[9C], wherein the one or more amino acid alterations are one or more amino acid alterations at positions selected from the group consisting of EU345, EU430, EU440, EU437, and EU248, wherein the numbers represent positions of substitutions according to EU numbering.
[9E] The antigen-binding molecule or the complex thereof of any one of [9]-[9D], wherein the one or more amino acid alterations are the combination E345R/E430G/S440Y or the combination T437R/K248E, wherein the numbers represent positions of substitutions according to EU numbering.
[9F] The antigen-binding molecule or the complex thereof of any one of [9]-[9E], further comprising a cell surface antigen-binding domain and a cytosolic antigen-binding domain.
[9G] The antigen-binding molecule or the complex thereof of any one of [9]-[9F] that has an elevated cytosol-penetrating ability as compared to an antigen-binding molecule comprising a parent Fc region which does not comprise the one or more amino acid alterations.
[9H] The antigen-binding molecule of any one of [9]-[9G], wherein the Fc region further comprises a modification that enhances association of a first Fc subunit and a second Fc subunit.
[9I] A method of potentiating cytosol-penetrating ability of a cytosol-penetrating antigen-binding molecule as compared to a parent cytosol-penetrating antigen-binding molecule, comprising introducing into an Fc region one or more amino acid alterations for enhancing formation of a multimer, wherein the parent cytosol-penetrating antigen-binding molecule comprises a parent Fc region that does not comprise the one or more amino acid alterations.
[9J] A method of producing a cytosol-penetrating antigen-binding molecule, wherein the method comprises introducing one or more amino acid alterations into an Fc region of a parent cytosol-penetrating antigen-binding molecule,
   wherein the alterations enhance formation of a multimer of the cytosol-penetrating antigen-binding molecule as compared to the parent cytosol-penetrating antigen-binding molecule, and
   wherein cytosol-penetrating ability of the cytosol-penetrating antigen-binding molecule is potentiated as compared to the parent cytosol-penetrating antigen-binding molecule.
[9K] The method of [9J], further comprising
   (a) obtaining an expression vector that comprises a gene encoding the cytosol-penetrating antigen-binding molecule produced by the method of [9J] and an operably linked suitable promoter,
   (b) introducing the vector into a host cell and culturing the host cell to allow production of the cytosol-penetrating antigen-binding molecule, and
   (c) recovering the cytosol-penetrating antigen-binding molecule from the host cell culture.
[9L] A method of screening for a cytosol-penetrating antigen-binding molecule, comprising
   (a) providing a parent cytosol-penetrating antigen-binding molecule comprising an Fc region,
   (b) obtaining a candidate molecule comprising an altered Fc region by introducing one or more amino acid alterations into the Fc region of the parent cytosol-penetrating antigen-binding molecule,
   (c) determining whether the candidate molecule forms a multimer,
   (d) identifying the candidate molecule as a suitable molecule when the candidate molecule forms more multimers as compared to the parent cytosol-penetrating antigen-binding molecule,
      wherein cytosol-penetrating ability of the cytosol-penetrating antigen-binding molecule is potentiated as compared to the parent cytosol-penetrating antigen-binding molecule.
[10] The antigen-binding molecule or the complex thereof of any one of [1]-[9H], wherein the region having cytosol-penetrating ability does not bind to an antigen expressed on the cell surface that is different from the cell surface antigen.
[11] The antigen-binding molecule or the complex thereof of any one of [1]-[9H] and [10], wherein the cell surface antigen is an antigen expressed specifically on a target cell, and wherein the antigen-binding molecule or the complex thereof is delivered specifically into cytosol of the target cell.
[11A] The antigen-binding molecule or the complex thereof of [11], which is substantially not delivered to cells that do not express the cell surface antigen.
[11B] A nucleic acid encoding the antigen-binding molecule or the complex thereof of any one of [1]-[9H] and [10]-[11A].
[11C] A vector comprising the nucleic acid of [11B].
[11D] A cell comprising the nucleic acid of [11B] or the vector of [11C].
[11E] A method of producing the antigen-binding molecule or the complex thereof of any one of [1]-[9H] and [10]-[11A], comprising culturing the cell of [11D].
[12] A pharmaceutical composition comprising the antigen-binding molecule or the complex thereof of any one of [1]-[9H] and [10]-[11A] and a pharmaceutically acceptable carrier.
[13] A method of delivering the antigen-binding molecule or the complex thereof of any one of [1]-[9H] and [10]-[11A] specifically into cytosol of a target cell, wherein the cell surface antigen is an antigen expressed specifically on the target cell.
[13A] The method of [13], comprising contacting the antigen-binding molecule or the complex thereof of any one of [1]-[9H] and [10]-[11A] with the target cell.
[13B] The method of [13] or [13A], wherein the method substantially does not deliver the antigen-binding molecule to cells that do not express the cell surface antigen.
[14] A method of removing, suppressing, or activating a cytosolic antigen in a target cell-specific manner by using the antigen-binding molecule or the complex thereof of any one of [1]-[9H] and [10]-[11A], wherein the cell surface antigen is an antigen expressed specifically on the target cell.
[14A] The method of [14], comprising contacting the antigen-binding molecule or the complex thereof of any one of [1]-[9H] and [10]-[11A] with the target cell.
[14B] The method of [14] or [14A], wherein the method substantially does not remove, suppress, or activate the cytosolic antigen of cells that do not express the cell surface antigen.
[15] A pharmaceutical composition for diagnosing, preventing, or treating a disease in a subject, wherein a diseased cell expresses the cell surface antigen and the cytosolic antigen to which the antigen-binding molecule or the complex thereof of any one of [1]-[9H] and [10]-[11A] specifically binds, and wherein the pharmaceutical composition comprises the antigen-binding molecule or the complex thereof of any one of [1]-[9H] and [10]-[11A] and a pharmaceutically acceptable carrier.
[15A] The pharmaceutical composition of [15] having reduced side-effect arising from the removal, suppression, or activation of the cytosolic antigen in cells that do not express the cell surface antigen, as compared to an antigen-binding molecule or a complex thereof which does not comprise a region that binds to the cell surface antigen.
[15B] A method of diagnosing, preventing, or treating a disease in a subject, wherein a diseased cell expresses the cell surface antigen and the cytosolic antigen to which the antigen-binding molecule or the complex thereof of any one of [1]-[9H] and [10]-[11A] specifically binds, the method comprising administering the antigen-binding molecule or the complex thereof of any one of [1]-[9H] and [10]-[11A].
[16] An antigen-binding molecule comprising a cell surface antigen-binding domain and a cytosol-penetrating domain, which is conjugated to a heterologous moiety.
[16A] The antigen-binding molecule of [16], which does not bind to an antigen expressed on the cell surface that is different from aforesaid cell surface antigen.
[16B] The antigen-binding molecule of [16], wherein the cytosol-penetrating domain do not bind to an antigen expressed on the cell surface that is different from aforesaid cell surface antigen.
[16C] The antigen-binding molecule of any one of [16]-[16B], wherein the antigen-binding molecule is a multifunctional antibody or a multifunctional antibody derivative.
[16D] The antigen-binding molecule of any one of [16]-[16C], wherein the antigen-binding molecule is a bifunctional antibody or a bifunctional antibody derivative.
[16E] The antigen-binding molecule of any one of [16]-[16D], wherein the heterologous moiety is a peptide, nucleic acid, chemotherapeutic agent or drug, growth inhibitory agent, toxin (e.g., protein toxin, enzymatically active toxin of bacterial, fungal, plant, or animal origin, or a fragment thereof), or radioactive isotope.
[16F] The antigen-binding molecule of any one of [16]-[16E], wherein the heterologous moiety is a peptide.
[17] An antigen-binding molecule comprising a first and a second Fab regions, wherein
   (a) the first Fab region binds specifically to a cell surface antigen, and
   (b) the second Fab region has cytosol-penetrating ability, and wherein the antigen-binding molecule is conjugated to a heterologous moiety.
[17A] The antigen-binding molecule of [17], further comprising an Fc region.
[17B] The antigen-binding molecule of [17] or [17A], wherein the Fc region comprises a modification that enhances association of a first Fc subunit and a second Fc subunit.
[17C] The antigen-binding molecule of any one of [17] to [17B], wherein the heterologous moiety is a peptide moiety.
[17D] A nucleic acid encoding the antigen-binding molecule of any one of [16]-[17C].
[17E] A vector comprising the nucleic acid of [17D].
[17F] A cell comprising the nucleic acid of [17D] or the vector of [17E].
[17G] A method of producing the antigen-binding molecule of any one of [16]-[17C], comprising culturing the cell of [17F].
[17H] A pharmaceutical composition comprising the antigen-binding molecule of any one of [16]-[17C] and a pharmaceutically acceptable carrier.
[17I] A method of delivering the heterologous moiety specifically into cytosol of a target cell, the method comprising contacting the antigen-binding molecule of any one of [16]-[17C] with the target cell.
[17J] The method of [17I], wherein the method substantially does not deliver the heterologous moiety to cells that do not express the cell surface antigen.
[18] A peptide comprising a mitochondrial outer membrane protein and a first fragment of a luminescent protein,
   wherein the first fragment and a second fragment of the luminescent protein together contain the full complement of the luminescent protein.
[18A] The peptide of [18], wherein each of the first fragment and the second fragment of the luminescent protein does not have the feature of exhibiting luminescence, but the luminescence-exhibiting feature is recovered when the first fragment of the luminescent protein comprised in the peptide binds to the second fragment of the luminescent protein.
[18B] The peptide of [18] or [18A] wherein the mitochondrial outer membrane protein is an additional mitochondrial kinase anchoring protein 1 (AKAP1).
[18C] The peptide of any one of [18] to [18B], wherein AKAP1 protein comprises an amino acid sequence of SEQ ID NO: 61.
[18D] The peptide of any one of [18] to [18C], wherein the luminescent protein comprises an amino acid sequence of SEQ ID NO: 64.
[18E] The peptide of any one of [18] to [18D], wherein the first fragment of the luminescent protein comprises an amino acid sequence of SEQ ID NO: 62, and the second fragment of the luminescent protein comprises an amino acid sequence of SEQ ID NO: 63.
[18F] The peptide of any one of [18] to [18E], which further comprises a green fluorescent protein (GFP) or its variant.
[18G] The peptide of [18F], wherein the GFP variant comprises an amino acid sequence of SEQ ID NO: 65.
[18H] The peptide of any one of [18] to [18G], which is for use in detecting a target molecule in cytosol with lower background signal compared with a peptide which is the same as any one of [18] to [18G] except that the peptide does not comprise the mitochondrial outer membrane protein, wherein the target molecule conjugated with the second fragment of the luminescent protein.
[19] A nucleic acid encoding the peptide of any one of [18] to [18H].
[19A] A vector comprising the nucleic acid of [19].
[19B] A cell comprising the nucleic acid of [19] or the vector of [19A].
[19C] A kit comprising the peptide of any one of [18] to [18H], the nucleic acid of [19], the vector of [19A], the cell of [19B].
[20] A split protein system, comprising
   (i) a peptide comprising a mitochondrial outer membrane protein and a first fragment of a luminescent protein, and
   (ii) a second fragment of the luminescent protein,
      wherein the first fragment and the second fragment of the luminescent protein together contain the full complement of the luminescent protein.
[20A] The split protein system of [20], wherein each of the first fragment and the second fragment of the luminescent protein does not have the feature of exhibiting luminescence, but the luminescence-exhibiting feature is recovered when the first fragment of the luminescent protein comprised in the peptide binds to the second fragment of the luminescent protein.
[20B] The split protein system of [20] or [20A] wherein the mitochondrial outer membrane protein is an additional mitochondrial kinase anchoring protein 1 (AKAP1).
[20C] The split protein system of any one of [20] to [20B], wherein AKAP1 protein comprises an amino acid sequence of SEQ ID NO: 61.
[20D] The split protein system of any one of [20] to [20C], wherein the luminescent protein comprises an amino acid sequence of SEQ ID NO: 64.
[20E] The split protein system of any one of [20] to [20D], wherein the first fragment of the luminescent protein comprises an amino acid sequence of SEQ ID NO: 62, and the second fragment of the luminescent protein comprises an amino acid sequence of SEQ ID NO: 63.
[20F] The split protein system of any one of [20] to [20E], wherein the peptide of (i) further comprises a green fluorescent protein (GFP) or its variant.
[20G] The split protein system of [20F], wherein the GFP variant comprises an amino acid sequence of SEQ ID NO: 65.
[20F] The split protein system of any one of [20] to [20G], which is for use in detecting a target molecule in cytosol with lower background signal compared with a split protein system which is the same as any one of [20]
[20G] except that the peptide of (i) does not comprise the mitochondrial outer membrane protein, wherein the target molecule conjugated with the second fragment of the luminescent protein.
[21] A method for detecting a cytosol-penetrating ability of a target molecule, which comprises
   (i) providing a cell comprising a nucleic acid encoding a peptide comprising a mitochondrial outer membrane protein and a first fragment of a luminescent protein,
   (ii) providing a target molecule conjugated with a second fragment of the luminescent protein,
   (iii) contacting the cell of (i) and the target molecule of (ii), and
   (iv) detecting luminescence signal in the cell of (iii),
      wherein the first fragment and the second fragment of the luminescent protein together contain the full complement of the luminescent protein.
[21A] The method of [21], wherein each of the first fragment and the second fragment of the luminescent protein does not have the feature of exhibiting luminescence, but the luminescence-exhibiting feature is recovered when the first fragment of the luminescent protein comprised in the peptide binds to the second fragment of the luminescent protein.
[21B] The method of [21] or [21A] wherein the mitochondrial outer membrane protein is an additional mitochondrial kinase anchoring protein 1 (AKAP1).
[21C] The method of any one of [21] to [21B], wherein AKAP1 protein comprises an amino acid sequence of SEQ ID NO: 61.
[21D] The method of any one of [21] to [21C], wherein the luminescent protein comprises an amino acid sequence of SEQ ID NO: 64.
[21E] The method of any one of [21] to [21D], wherein the first fragment of the luminescent protein comprises an amino acid sequence of SEQ ID NO: 62, and the second fragment of the luminescent protein comprises an amino acid sequence of SEQ ID NO: 63.
[21F] The method of any one of [21] to [21E], wherein the peptide of (i) further comprises a green fluorescent protein (GFP) or its variant.
[21G] The method of [21F], wherein the GFP variant comprises an amino acid sequence of SEQ ID NO: 65.
[21H] The method of any one of [21] to [21G], wherein the background signal is lower compared with a method which is the same as any one of [21] to [21G] except that the peptide of (i) does not comprise the mitochondrial outer membrane protein.
[21J] A method of screening a cytosol-penetrating antigen binding molecule, which comprises
   (i) providing a test antigen binding molecule,
   (ii) detecting a cytosol-penetrating ability of the test antigen binding molecule of (i) by the method according to any one of [21] to [21H],
      wherein the cytosol-penetrating ability of the test antigen binding molecule is detected, the test antigen binding molecule is identified as a cytosol-penetrating antigen binding molecule.

### BRIEF DESCRIPTION OF DRAWINGS

FIG. 1 is a schematic drawing showing the concept of the antigen-binding molecules of the present disclosure. In FIG. 1, an antigen-binding molecule in the form of a bifunctional antibody is shown for example to illustrate the concept of the target cell-specific delivery of the antigen-binding molecules of the present disclosure.

FIG. 2 is a schematic drawing showing molecular forms of multivalent antibodies, as examples of molecular forms for the antigen-binding molecules of the present disclosure.

FIG. 3 shows the results of evaluating accumulation of bifunctional antibodies in the cytosol of CHO cells or IL6R+CHO cells by BirA assay, which cells were incubated for 6 hours in a culture medium containing each of the bifunctional antibodies indicated in the figure. The band displayed at the position marked with the arrowhead below 66 kDa is the luminescent signal from HRP, showing the biotin-labeled antibody heavy chain by BirA.

FIG. 4 shows the results of evaluating accumulation of antibodies in the cytosol of CHO cells or IL6R+CHO cells by imaging analysis using fluorescent microscopy, which cells were incubated for 1 hour in a culture medium containing each of the bifunctional antibodies indicated in the figure. FIG. 4(1) is a graph plotting values of fluorescent signals from detected antibodies, normalized by the number of cells. The vertical axis is the fluorescence signal (A.U.: Arbitrary Unit). FIG. 4(2) is images from fluorescent microscopy.

FIG. 5 shows the results of evaluating accumulation of antibodies in the cytosol of IL6R+CHO cells by BirA assay, which cells were incubated for 6 hours in a culture medium containing the antibodies at each concentration indicated in the figure. The band displayed at the position marked with the arrowhead below 40 kDa is the luminescent signal from HRP, showing the biotin-labeled antibody light chain by BirA.

FIG. 6 shows the results of evaluating accumulation of antibodies in the cytosol of CHO cells or Hela cells by imaging analysis using fluorescent microscopy, which cells were incubated for 1 hour in a culture medium adjusted at pH7.4 or pH5.5 and containing each of the antibodies indicated in the figure. FIG. 6(1) is a graph plotting values of fluorescent signals from detected antibodies, normalized by the number of cells. The vertical axis is the fluorescence signal (A.U.: Arbitrary Unit). FIG. 6(2) is images from fluorescent microscopy.

FIG. 7 is a schematic drawing showing exemplary molecular forms of the antigen-binding molecules of the present disclosure. The antigen-binding molecules comprise a first and a second Fab regions.

FIG. 8 is a schematic drawing showing exemplary molecular forms of the antigen-binding molecules of the present disclosure. The antigen-binding molecules comprise a first and a second Fab regions and a single-chain unit.

FIG. 9 is a schematic drawing showing exemplary molecular forms of the antigen-binding molecules of the present disclosure. The antigen-binding molecules comprise a first and a second single-domain antibody variable regions and a single-chain unit.

FIG. 10 is a schematic drawing showing exemplary molecular forms of the antigen-binding molecules of the present disclosure. The antigen-binding molecules have the molecular form of DVD-Ig (registered trademark).

FIG. 11 is a schematic drawing showing exemplary molecular forms of the antigen-binding molecules of the present disclosure. The antigen-binding molecules comprise a first, a second, and a third Fab regions.

FIG. 12 is a schematic drawing showing exemplary molecular forms of the antigen-binding molecules of the present disclosure. The antigen-binding molecules comprise a first, a second, a third, and a fourth Fab regions.

FIG. 13 shows the difference of split Nanoluc signals of EGFP-Nanoluc LgBiT and AKAP-EGFP-Nanoluc LgBiT HeLa cells.

FIG. 14A shows the results of split-Nanoluc assay to assess cytosol trafficking of bifunctional antibodies for 3D8 antibody in AKAP-EGFP-Nanoluc LgBiT HeLa cells. FIG. 14B shows the results of split-Nanoluc assay assessed cytosol trafficking of bifunctional antibodies for 2C10 antibody in AKAP-EGFP-Nanoluc LgBiT HeLa cells.

FIG. 15 shows the results of biotin ligase (BirA) assay to assess cytosol trafficking of bifunctional antibodies in HeLa/BirA/IL6R cells.

FIG. 16 shows the results of biotin ligase (BirA) assay to assess cytosol trafficking of bifunctional antibodies in HeLa/ASGPR/BirA cells.

FIG. 17 shows the results of imaging analysis for cytosol trafficking of cell penetrating antibodies and receptor binding antibodies in bivalent format, in HeLa/BirA cells (FIG. 17A), in HeLa/BirA/IL6R cells (FIG. 17B), and in HeLa/ASGPR/BirA cells (FIG. 17C).

FIG. 18A shows the results of imaging analysis for cytosol trafficking of bifunctional antibodies in HeLa/BirA cells.

FIG. 18B shows the results of imaging analysis for cytosol trafficking of bifunctional antibodies in HeLa/BirA/IL6R cells.

FIG. 18C shows the results of imaging analysis for cytosol trafficking of bifunctional antibodies in HeLa/ASGPR/BirA cells.

FIG. 19 shows cytosolic fluorescence detected in the imaging analysis for cytosol trafficking of bifunctional antibodies, in HeLa/BirA cells (FIG. 19A), in HeLa/BirA/IL6R (FIG. 19B), and in HeLa/ASGPR/BirA cells (FIG. 19C).

FIG. 20 shows the results of imaging analysis for cytosol delivery of cargo molecule by bifunctional antibodies in HeLa/BirA cells (FIG. 20A), in HeLa/BirA/IL6R cells (FIG. 20B), and in HeLa/ASGPR/BirA cells (FIG. 20C).

FIG. 21 shows cytosolic fluorescence detected in the imaging analysis for cytosol delivery of cargo molecule by bifunctional antibodies, in HeLa/BirA cells (FIG. 21A), in HeLa/BirA/IL6R (FIG. 21B), and in HeLa/ASGPR/BirA cells (FIG. 21C).

### DESCRIPTION OF EMBODIMENTS

### 1. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “antigen-binding molecule” refers, in its broadest sense, to a molecule that specifically binds to an antigenic determinant. In one embodiment, the antigen-binding molecule is an antibody, antibody fragment, or antibody derivative.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD) or “amount of bound analyte per unit amount of ligand”. Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Binding activity” refers to the strength of the sum total of noncovalent interactions between one or more binding sites of a molecule (e.g., an antibody) and its binding partner (e.g., an antigen). Herein, “binding activity” is not strictly limited to a 1:1 interaction between members of a binding pair (e.g., antibody and antigen). For example, when a member of a binding pair is capable of both monovalent binding and multivalent binding, the binding activity is the sum of each binding strength. The binding activity of a molecule X for its partner Y can generally be represented by the dissociation constant (KD) or “amount of bound analyte per unit amount of ligand”. Specific illustrative and exemplary embodiments for measuring binding affinity are described in the following.

The term “antibody” herein is used in the broadest sense and encompasses various antibody structures, including but not limited to monoclonal antibodies, polyclonal antibodies, multifunctional antibodies, multispecific antibodies (e.g., bispecific antibodies), and antibody fragments so long as they exhibit the desired antigen-binding activity.

An “agonist” antigen-binding molecule or “agonist” antibody, as used herein, is an antibody which significantly potentiates a biological activity of the antigen it binds.

A “blocking” antigen-binding molecule or “blocking” antibody or an “antagonist” antigen-binding molecule or “antagonist” antibody, as used herein, is an antibody which significantly inhibits (either partially or completely) a biological activity of the antigen it binds.

An “antibody fragment” refers to a molecule other than an intact antibody that comprises a portion of an intact antibody. Examples of antibody fragments include but are not limited to Fv (VH and VL), Fab, Fab′, Fab′-SH, F(ab′)2; diabodies; linear antibodies; single-chain antibody molecules (e.g. scFv); and multifunctional antibodies and multispecific antibodies that are formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., 211At, 131I, 125I, 90Y, 186Re, 188Re, 153Sm, 212Bi, 32P, 212Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Herein, amino acid alterations or substitutions within an Fc region or a constant region may be represented by the combination of the EU numbering system and amino acids. For example, 5424N stands for substitution at position 424 in EU numbering from serine (Ser) to asparagine (Asn). EU424N stands for substitution at position 424 in EU numbering from an amino acid (any type) to asparagine (Asn).

The term “Fc receptor” or “FcR” refers to a receptor that binds to the Fc region of an antibody. An Fc receptor is a protein on the surface of immune cells such as natural killer cells, macrophages, neutrophils and mast cells. Fc receptors bind to the Fc (crystallizable fragment) region of antibodies attached to infected cells or invading pathogens, stimulate phagocytes or cytotoxic cells, and thereby destroy microorganisms or infected cells by antibody-mediated phagocytosis or antibody-dependent cell-mediated cytotoxicity. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

The term “Fc region-comprising antibody” herein refers to an antibody that comprises an Fc region. The C-terminal lysine (residue 447 according to the EU numbering system) or C-terminal glycine-lysine (residues 446-447) of the Fc region may be removed, for example, during purification of the antibody or by recombinant engineering of the nucleic acid encoding the antibody. Accordingly, a composition comprising an antibody having an Fc region according to present disclosure can comprise an antibody with G446-K447, with G446 and without K447, with all G446-K447 removed, or a mixture of three types of antibodies described above.

The terms “a subunit of an Fc domain” and “an Fc subunit” herein indicate one of the two polypeptides forming a dimeric Fc region, in other words, a polypeptide comprising a C-terminal constant region of an immunoglobulin heavy chain that is capable of stable self-association. For example, a subunit of an Fc domain of IgG comprises IgG CH2- and IgG CH3-constant domains.

“Modification that enhances association of a first Fc subunit and a second Fc subunit” is engineering of a peptide backbone or post-translational modifications on an Fc domain subunit that reduce or prevent association of a polypeptide with the same species of polypeptide, which polypeptide comprises the Fc domain subunit capable of forming a homodimer. The association-enhancing modifications used herein include separate modifications each one of which is made to one of the two Fc domain subunits for which association is desired (i.e., a first and a second subunits of an Fc domain), the modifications being complementary to each other such that it enhances association of the two Fc domain subunits. For example, the association-enhancing modifications may alter the structure or electric charge of one or both in the Fc domain for making the association thereof sterically or electrostatically desirable. Therefore, (hetero-)dimerization occurs between a polypeptide comprising the first Fc domain subunit and a polypeptide comprising the second Fc domain subunit, which polypeptides may be nonidentical in that further components (e.g., an antigen-binding portion) fused to each subunit are not identical. In some embodiments, the association-enhancing modifications comprise amino acid mutations, in particular amino acid substitutions, within a Fc domain. In a certain embodiment, the association-enhancing modifications comprise different amino acid mutations, in particular amino acid substitutions, in each of the two subunits of a Fc domain.

In one embodiment, the aforementioned modifications are so-called “knob into hole” modifications, including a knob modification in one of the two subunits of an Fc domain and a hole modification in the other of the two subunits of the Fc domain. The knob-into-hole technology is described, for example, in U.S. Pat. Nos. 5,731,168; 7,695,936; Ridgway et al., Prot Eng 9, 617-621 (1996); and Carter, J Immunol Meth 248, 7-15 (2001). Generally, this method includes introducing a projection (knob) at the interface of a first polypeptide and a corresponding cavity (hole) at the interface of a second polypeptide so that the projection can pack into the cavity to enhance heterodimer formation while to prevent homodimer formation. The projection can be constructed by substituting a small side chain of an amino acid from the interface of the first polypeptide by a larger side chain (e.g., thyrosin or tryptophan). A complementary cavity of the same or smaller size with the projection can be created at the interface of the second polypeptide by substitution a large amino acid side chain by a smaller one (e.g., alanine or threonine).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding a cytosol-penetrating antigen-binding molecule” refers to one or more nucleic acid molecules encoding a cytosol-penetrating antigen-binding molecule, including nucleic acid molecules carried on a single vector or separate vectors, and nucleic acid molecules present at a single site or a plurality of sites within a host cell. In the case that TR cytosol-penetrating antigen-binding molecule is an antibody, the “isolated nucleic acid encoding a cytosol-penetrating antigen-binding molecule” refers to one or more nucleic acid molecules encoding the heavy chain and the light chain (or fragments thereof) of the antibody, including nucleic acid molecules carried on a single vector or separate vectors, and nucleic acid molecules present at a single site or a plurality of sites within a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (fE) and lambda (fE), based on the amino acid sequence of its constant domain.

The term “cytosol-penetrating antigen-binding molecule” refers to an antigen-binding molecule that can penetrate into the cytosol of a living cell. The mode for the penetration into the cytosol is not particularly limited, and the antigen-binding molecule may be uptaken via the endocytosis process and then go through the endosome escape, or may undergo other processes. Examples other than the endosome escape include the case where an antigen-binding molecule directly penetrates through the cell membrane. The same antigen-binding molecule may penetrate into cytosol by both modes, i.e. the endosome escape and the direct penetration of the cell membrane. Antigen-binding molecules capable of penetrating into cytosol via methods other than endosome escape are reported (e.g., WO 2013102659). In one embodiment, the cytosol-penetrating antigen-binding molecules are antibodies or antibody derivatives. In another embodiment, the cytosol-penetrating antigen-binding molecules comprise a cytosol-penetrating domain.

The terms “cytosol-penetrating domain” and “region having cytosol-penetrating ability” are used interchangeably to refer to a domain that allows for penetration into the cytosol of a living cell. The mode for the penetration into the cytosol is not particularly limited, and may be through the uptake via the endocytosis process and following endosome escape, or may through other processes. The mode for the escape from endosomes is not particularly limited. For example, transfer into cytosol is achieved by the cytosol-penetrating domain in an endosome (acidic pH) interacting with the endosome membrane to create a pore on the endosome membrane. Examples other than the endosome escape include the case where an antigen-binding molecule directly penetrates through the cell membrane. The same antigen-binding molecule may penetrate into cytosol by both modes, i.e. the endosome escape and the direct penetration of the cell membrane. In one embodiment, the cytosol-penetrating domain has one or both of the endosome-escaping ability and cell membrane-permeating ability. In one embodiment, the cytosol-penetrating domain does not have an ability to be uptaken by endocytosis. In another embodiment, the cytosol-penetrating domain has an ability to be uptaken by endocytosis. The cytosol-penetrating domain may be of any type as long as it has cytosol-penetrating ability, but in one preferred embodiment, the cytosol-penetrating domain is an antibody or a fragment thereof, a peptide moiety, or a nucleic acid moiety. In the case that the cytosol-penetrating domain is an antibody or a fragment thereof, the cytosol-penetrating domain includes the whole or a portion of a combination of an antibody light chain variable region (VL) and an antibody heavy chain variable region (VH); or a single-domain antibody variable region, such as a heavy-chain antibody variable region (Variable region of Heavy chain of Heavy chain antibody: VHH), an antibody heavy chain variable region (VH), an antibody light chain variable region (VL), and a variable region of an immunoglobulin new antigen receptor (IgNAR) (VNAR). Examples of an antibody or a fragment thereof having cytosol-penetrating ability include, for example, Cytotransmab comprising a light chain variable region which exhibits cytosol-penetrating ability (Nat Commun. 2017 May 10; 8:15090). Other examples of cytosol-penetrating domains are “scFv (single chain Fv)”, “single chain antibody”, “Fv”, “scFv2 (single chain Fv 2)”, “Fab” or “F(ab)2”, etc. Examples of peptide moieties as cytosol-penetrating domain include cell-penetrating peptides (CPP), protein transduction domains (PTD), etc. (see, for example, WO 2017156630). Examples of nucleic acid moieties as cytosol-penetrating domain include oligo-nucleic acids having a phosophorothioate backbone (see, for example, WO 2015031837). In one embodiment, the cytosol-penetrating antigen-binding molecule is a fusion or a complex of an antigen-binding molecule having no cytosol-penetrating ability and a cytosol-penetrating domain.

Whether a domain of interest is a cytosol-penetrating domain or not (whether the domain of interest has cytosol-penetrating ability or not) can be checked by various methods known to those skilled in the art. For example, assessment can be made by fusing the domain of interest with a molecule having no cytosol-penetrating ability (e.g., an antigen-binding molecule), contacting the fused molecule with a cell, and then seeing if the fused molecule is detected in the cytosol of the cell. Various methods are known for detecting the presence of a molecule of interest in cytosol, and, for example, BirA assay and imaging analysis using fluorescence microscopy described in the Examples below are known.

In one embodiment, the cytosol-penetrating domain may interact with a cell surface antigen and/or a cytosolic antigen.

“Cytosol-trafficking” ability of an antigen-binding molecule means the ability of the antigen-binding molecule to traffic to the cytosol, and the term “penetrate”, “traffic” or “traffick” are used interchangeably.

“Fuse/fusing/fusion” means that components (e.g., a Fab region and an Fc domain subunit) are linked together directly or by peptide bond via one or more peptide linkers.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules of the present disclosure are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The phrase “substantially reduced”, “substantially increased”, or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., KD values).

The term “substantially similar”, “substantially unchanged”, or “substantially the same,” as used herein, refers to a sufficiently high degree of similarity between two numeric values (for example, one associated with an antigen-binding molecule of the present disclosure and the other associated with a reference/comparator antigen-binding molecule), such that one of skill in the art would consider the difference between the two values to be of little or no biological and/or statistical significance within the context of the biological characteristic measured by said values (e.g., KD values).

The expression “substantially not detected” used herein means that something is on or below the detection limit in standard detection techniques known to those skilled in the art (e.g., Western blotting, capillary immunoassay, imaging by fluorescent microscopy, etc.), which means that the something is totally not detected or, even when detected the detection level is near the background or noises. Specifically, the antigen-binding molecules of the present disclosure are said as being substantially not detected, for example, when measurements for the antigen-binding molecules of the present disclosure are substantially similar to or substantially the same as (e.g., there is no statistical significance from) the measurements for negative controls.

The expression “substantially not delivered” means that the delivery of something is substantially not detected in standard detection techniques for detecting the delivery. For example, for the delivery into the cytosol of a cell, an antigen-binding molecule is said as being substantially not delivered into the cytosol of a cell when the amount of the antigen-binding molecule of the present disclosure or a complex thereof in the cytosol of a cell having been contacted with the antigen-binding molecule or a complex thereof is substantially similar to or substantially the same as (e.g., there is no statistical significance from) the amount in the cytosol of a cell for which no such contact has been made.

Similarly, the expression substantially does not remove, suppress, or activate means that the removal, suppression, activation is substantially not detected in standard detection techniques for detecting them. For example, for the removal, suppression, activation of a cytosolic antigen, the cytosolic antigen is said as being substantially not removed, suppressed, or activated when the measurements in the case of using the antigen-binding molecule of the present disclosure or a complex thereof are substantially similar to or substantially the same as (e.g., there is no statistical significance from) the measurements for controls.

### II. Compositions and Methods

In one aspect, the present disclosure is based, in part, on the finding that multifunctional antigen-binding molecules comprising a cytosol-penetrating antibody or a fragment of the antibody, an antibody binding to a cell surface antigen or a fragment of the antibody, and/or an antibody binding to a cytosolic antigen or a fragment of the antibody in combination are delivered into cytosol in a target cell-specific manner. In certain embodiments, antigen-binding molecules comprising a cell surface antigen-binding domain, a cytosolic antigen-binding domain, and a cytosol-penetrating domain are provided. The antigen-binding molecules of the present disclosure are useful, for example, for diagnosis, prevention, or treatment of a disease that arises due to the cytosolic antigen.

### A. Cytosol-Penetrating Antigen-Binding Molecules

In one aspect, the antigen-binding molecules of the present disclosure are cytosol-penetrating antigen-binding molecules. In one embodiment, the cytosol-penetrating antigen-binding molecules of the present disclosure comprise a cell surface antigen-binding domain, a cytosolic antigen-binding domain, and a cytosol-penetrating domain. In one preferred embodiment, the cytosol-penetrating antigen-binding molecules of the present disclosure do not bind to antigens that are expressed on the surface of a cell and different from the aforementioned cell surface antigen. In one preferred embodiment, the cytosolic antigen-binding domain and/or the cytosol-penetrating domain (i) do not bind to antigens that are expressed on the surface of a cell and different from the aforementioned cell surface antigen; or (ii) do not bind to any antigens expressed on the surface of a cell.

It is known that one already existing cell-penetrating antibody Tmab4 (heavy chain: 3D8, light chain: hT4VL) is uptaken into intracellular endosomes by cell surface heparin sulfate proteoglycan (HSPG)-dependent endocytosis, goes through structural changes of the light chain under acidic pH in the endosomes and comes to interact with the endosome membrane to create pores on the membrane, and thereby achieves translocation into the cytosol (J Control Release. 2016 Aug. 10; 235:165-175). It is also reported that the antibody made to have reduced binding toward HSPG (Tmab4-03 (heavy chain: 3D8, light chain: hT4VL.03) has lost its ability to be uptaken by endocytosis, while maintaining pore-forming ability at pH5.5 (J Control Release. 2016 Aug. 10; 235:165-175). This means that Tmab4-03, in the form as it stands, cannot achieve translocation into cell in a cell-specific manner. In addition, it is expected that membrane deformation through interaction of Tmab4 with molecules on the endosome membrane is crucial for allowing pores to be formed in the endosome membrane (PNAS, 2011 October108; 41; 16883-16888). Therefore, in one embodiment, it is desirable that there are multivalent cytosol-penetrating domains that interact with the endosome membrane, in order to improve cytosol-penetrating ability.

On the other hand, as described above, Tmab4 goes through endocytosis via HSPG which is universally expressed on epithelial cells, and therefore, it is difficult to deliver cytosolic antigen-binding antibodies in a target cell-specific manner. In other words, it is expected that delivery of cytosolic antigen-binding antibodies to a target tissue upon systemic administration will be low. In view of these, in one embodiment, the present inventors arrived at the idea of using a monovalent cytosol-penetrating domain to suppress nonspecific uptake, adding a target cell surface-binding domain to improve target specificity, and, at the same time, creating conditions where the interaction of antibodies and endosome membrane occurs in a multivalent fashion, in order to keep or improve cytosol-penetrating ability.

Unlike the above-described already existing antibodies, in one embodiment, the cytosol-penetrating antigen-binding molecules of the present disclosure comprise a cell surface antigen-binding domain, cytosolic antigen-binding domain, and a cytosol-penetrating domain. Therefore, in one embodiment, for the cytosol-penetrating antigen-binding molecules of the present disclosure it is expected that it binds to a cell surface antigen on a target cell and thereby undergoes endocytosis in a target cell-specific manner, and then it translocates itself from the endosomes into the cytosol. In one embodiment, the cytosol-penetrating antigen-binding molecules of the present disclosure are more selectively delivered into the cytosol of target cells as compared to the already existing cytosol-penetrating antibodies. In one embodiment, a larger amount of the antigen-binding molecules is delivered to the cytosol of a target cell when a fixed amount of the cytosol-penetrating antigen-binding molecules of the present disclosure is administered or is contacted with a subject, as compared to the case of the same amount of the already existing cytosol-penetrating antibodies. In one embodiment, the cytosol-penetrating antigen-binding molecules of the present disclosure are delivered specifically into the cytosol of a target cell, while substantially not delivered to cells that do not express the cell surface antigen to which the antigen-binding molecules specifically bind. In one embodiment, when the cytosol-penetrating antigen-binding molecules of the present disclosure are used as pharmaceuticals, the cytosol-penetrating antigen-binding molecules exert stronger drug efficacy and/or cause lesser side effects, as compared to the already existing cytosol-penetrating antibodies. In one embodiment, pharmaceutical compositions comprising the cytosol-penetrating antigen-binding molecule of the present disclosure can exert drug efficacy with smaller amount of administration and/or lesser number of administrations as compared to pharmaceutical compositions comprising the already existing cytosol-penetrating antibody.

The term “antigen binding domain” herein refers to a portion of an antigen-binding molecule, the portion comprising a region that specifically binds and is complementary to the whole or a portion of an antigen. The antigen binding domain may bind only to a specific portion of an antigen if the antigen is of high molecular weight. That specific portion is called “epitope”. The antigen binding domain may be provided with one or more antibody variable domains. In one preferred embodiment, the antigen binding domain comprises the whole or a portion of a combination of an antibody light chain variable region (VL) and an antibody heavy chain variable region (VH); or a single-domain antibody variable region, such as a heavy-chain antibody variable region (VHH), an antibody heavy chain variable region (VH), an antibody light chain variable region (VL), and a variable region of an immunoglobulin new antigen receptor (IgNAR) (VNAR). Other examples of antigen binding domains are “scFv (single chain Fv)”, “single chain antibody”, “Fv”, “scFv2 (single chain Fv 2)”, “Fab” or “F(ab′)2”, etc.

The terms “single-domain antibody”, “single-domain antibody variable region”, “one-domain antibody”, and “one-domain antibody variable region” used herein are not limited by the structure as long as the domain can exert antigen binding activity by itself. It is known that a typical antibody, such as IgG antibodies for example, shows antigen binding activity in a state where a variable region is formed by the pairing of VH and VL, whereas, the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR (variable region of immunoglobulin new antigen receptor: IgNAR), and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention. The “humanized single-domain antibody” refers to a chimeric single-domain antibody comprising amino acid residues from non-human CDRs and amino acid residues from human FRs. In an embodiment, in the humanized single-domain antibody, all or substantially all CDRs correspond to those of a non-human antibody and all or substantially all FRs correspond to those of a human antibody. Even in the case that a part of FR residues does not correspond to those from a human antibody in a humanized antibody, it is considered as one example of the case where substantially all FRs correspond to those from a human antibody. For example, to humanize a VHH, an embodiment of single-domain antibodies, a part of the FR residues has to be residues that do not correspond to those in a human antibody (C Vincke et al., The Journal of Biological Chemistry 284, 3273-3284).

The single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta-Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

The expression “specifically bind/binds” used herein means that one of specifically binding molecules shows binding in the condition where it does not substantially bind to molecules other than its one or more binding partner molecules. This expression is also used when the antigen binding domain has specificity for a particular epitope out of a plurality of epitopes contained in an antigen. In the case that an epitope to which the antigen-binding domain binds is contained in a plurality of different antigens, an antigen-binding molecule having that antigen-binding domain can bind to various antigens which contain the epitope.

The “cell surface antigen-binding domain” in the antigen-binding molecule of the present disclosure can bind to an epitope that is present in an antigen expressed on the surface of a cell. The “cell surface antigen” represents an antigen structure that is expressed by a cell and is present on the surface of the cell such that an antigen-binding domain can access it. In one embodiment, the cytosol-penetrating antigen-binding molecules of the present disclosure comprise a cell surface antigen-binding domain, and therefore, are specifically incorporated into cells that express the cell surface antigen (target cells). This enables specific delivery of the cytosol-penetrating antigen-binding molecules to target cells. In one embodiment, the cell surface antigen-binding domain can be provided with one or more antibody variable domains, and may be in any form as exemplified above as “antigen binding domain”.

The “cytosolic antigen-binding domain” in the antigen-binding molecules of the present disclosure can bind to an epitope that is present in an antigen expressed in the cytosol. The “cytosolic antigen” represents an antigen structure that is expressed by a cell and is present in the cytosol. Cytosolic antigen may be a protein, a nucleic acid such as DNA or RNA, or a molecule expressed in organelle such as cell nucleus and mitochondria. The cytosolic antigen-binding molecule of the present disclosure comprises a cytosolic antigen-binding domain, and therefore, can bind to the cytosolic antigen in the cytosol of a target cell and thus can neutralize, inhibit, or activate, etc. the function of the antigen. In one embodiment, the cytosolic antigen-binding domain can be provided with one or more antibody variable domains, and may be in any form as exemplified above as “antigen binding domain”. In one further embodiment, the cytosolic antigen-binding domain may be enzymes or shRNAs, etc.

In one embodiment, when it is said that a cytosol-penetrating antigen-binding molecule is delivered into the cytosol in a target cell-specific manner, it means that a) the amount of the cytosol-penetrating antigen-binding molecule present in the cytosol of non-target cells at an arbitrary time point after allowing the cytosol-penetrating antigen-binding molecule to contact with the target cells and non-target cells is small or is substantially reduced as compared to b) the amount of the cytosol-penetrating antigen-binding molecule present in the cytosol of the target cells at the same time point. In one preferred embodiment, the cytosol-penetrating antigen-binding molecule of the present disclosure is substantially not detected from the cytosol of non-target cells after allowing the cytosol-penetrating antigen-binding molecule to contact with the non-target cells. In one preferred embodiment, the cells are those derived from Hela cell line, CHO cell line, MDCK cells, or HepG2 cell line, expressing or not expressing cell surface antigens. The amount of the cytosol-penetrating antigen-binding molecule to contact with the cells may be freely selected, but the amount of the cytosol-penetrating antigen-binding molecule for the above a) and the amount of the cytosol-penetrating antigen-binding molecule for the above b) are the same. The contact between the cytosol-penetrating antigen-binding molecules and cells can be made by any methods, including incubation.

In one embodiment, the amount of the cytosol-penetrating antigen-binding molecule present in the cytosol is measured after 0 hour, 0.25 hour, 0.5 hour, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, and/or 16 hours from the time of allowing the cytosol-penetrating antigen-binding molecule to contact with cells. The amount of the cytosol-penetrating antigen-binding molecule present in the cytosol may be measured only once or measured twice or more times after allowing the cytosol-penetrating antigen-binding molecule to contact with cells. By measuring the amount of the cytosol-penetrating antigen-binding molecules present in the cytosol several times, it is possible to observe over time changes in the amount of the cytosol-penetrating antigen-binding molecule present in the cytosol.

In one preferred embodiment, a) the amount of the cytosol-penetrating antigen-binding molecule present in the cytosol of non-target cells at an arbitrary time point after allowing the cytosol-penetrating antigen-binding molecule to contact with the target cells and non-target cells is 50% or less, 40% or less, 30% or less, 20% or less, 10% or less, or 5% or less of b) the amount of the cytosol-penetrating antigen-binding molecule present in the cytosol of the target cells at the same time point.

In one embodiment, when the amount of the cytosol-penetrating antigen-binding molecule present in the cytosol at an arbitrary time point is expressed by HRP luminescence signal or fluorescence signal or luminescence signal, a) the HRP luminescence signal intensity or fluorescence signal intensity or luminescence signal intensity by non-target cells at an arbitrary time point after allowing the cytosol-penetrating antigen-binding molecule to contact with the non-target cells and target cells is 0.5-fold or less, 0.4-fold or less, 0.3-fold or less, 0.2-fold or less, 0.1-fold or less, or 0.05-fold or less of b) the HRP luminescence signal intensity or fluorescence signal intensity or luminescence signal intensity by the target cells at the same time point. In one embodiment, when the method comprising cells expressing biotin ligase (BirA) (such as the method described in Example 4) is used, the amount of the cytosol-penetrating antigen-binding molecule present in the cytosol at an arbitrary time point can be expressed by HRP luminescence signal. In a different embodiment, when the imaging analysis (such as the method described in Example 5) is used, the amount of the cytosol-penetrating antigen-binding molecule present in the cytosol at an arbitrary time point can be expressed by fluorescence signal. In a different embodiment, when the split protein system (such as the method described in Example 9) is used, the amount of the cytosol-penetrating antigen-binding molecule present in the cytosol at an arbitrary time point can be expressed by luminescence signal.

In one working embodiment of the present invention, peptide linkers may be used to fuse the cell surface antigen-binding domain and the cytosol-penetrating domain, the cell surface antigen-binding domain and the cytosolic antigen-binding domain, the cytosol-penetrating domain and the cytosolic antigen-binding domain, the cell surface antigen-binding domain and an Fc subunit, the cytosol-penetrating domain and an Fc subunit, the cytosolic antigen-binding domain and an Fc subunit. In addition, peptide linkers may be used in the antigen binding molecules having molecular forms 1 to 7 described below, in order to fuse one Fab region to another Fab region and to fuse the Fab region to an Fc subunit. For example, amino acids freely selected from Arg, Ile, Gln, Glu, Cys, Tyr, Trp, Thr, Val, His, Phe, Pro, Met, Lys, Gly, Ser, Asp, Asn, Ala, etc., particularly, Gly, Ser, Asp, Asn, Ala, in particular, Gly and Ser, and especially Gly are included.

Suitable peptide linkers can be easily selected by those skilled in the art, and suitable ones may be selected from those with different lengths, such as 1 amino acid (Gly, etc.) to 21 amino acids, 2 amino acids to 15 amino acids, or 3 amino acids to 12 amino acids (such as 4 amino acids to 10 amino acids, 5 amino acids to 9 amino acids, 6 amino acids to 8 amino acids, or 7 amino acids to 8 amino acids).

Examples of peptide linkers include, but are not limited to, glycine polymers (G)n, glycine-serine polymers (including e.g., (GS)n, (GSGGS: SEQ ID NO: 10)n and (GGGS: SEQ ID NO: 1)n, wherein n is an integer of at least 1), glycine-alanine polymers, alanine-serine polymers, and other flexible linkers well known in conventional techniques.

Among them, glycine and glycine-serine polymers are receiving attention because these amino acids are relatively unstructured and easily function as neutral tethers between components.

Examples of the flexible linker consisting of the glycine-serine polymer include, but are not limited to,

Ser
Gly-Ser (GS)
Ser-Gly (SG)
Gly-Gly-Ser (GGS)
Gly-Ser-Gly (GSG)
Ser-Gly-Gly (SGG)
Gly-Ser-Ser (GSS)
Ser-Ser-Gly (SSG)
Ser-Gly-Ser (SGS)
Gly-Gly-Gly-Ser (GGGS: SEQ ID NO: 1)
Gly-Gly-Ser-Gly (GGSG: SEQ ID NO: 2)
Gly-Ser-Gly-Gly (GSGG: SEQ ID NO: 3)
Ser-Gly-Gly-Gly (SGGG: SEQ ID NO: 4)
Gly-Ser-Ser-Gly (GSSG: SEQ ID NO: 5)
Gly-Gly-Gly-Gly-Ser (GGGGS: SEQ ID NO: 6)
Gly-Gly-Gly-Ser-Gly (GGGSG: SEQ ID NO: 7)
Gly-Gly-Ser-Gly-Gly (GGSGG: SEQ ID NO: 8)
Gly-Ser-Gly-Gly-Gly (GSGGG: SEQ ID NO: 9)
Gly-Ser-Gly-Gly-Ser (GSGGS: SEQ ID NO: 10)
Ser-Gly-Gly-Gly-Gly (SGGGG: SEQ ID NO: 11)
Gly-Ser-Ser-Gly-Gly (GSSGG: SEQ ID NO: 12)
Gly-Ser-Gly-Ser-Gly (GSGSG: SEQ ID NO: 13)
Ser-Gly-Gly-Ser-Gly (SGGSG: SEQ ID NO: 14)
Gly-Ser-Ser-Ser-Gly (GSSSG: SEQ ID NO: 15)
Gly-Gly-Gly-Gly-Gly-Ser (GGGGGS: SEQ ID NO: 16)
Ser-Gly-Gly-Gly-Gly-Gly (SGGGGG: SEQ ID NO: 17)
Gly-Gly-Gly-Gly-Gly-Gly-Ser
(GGGGGGS: SEQ ID NO: 18)
Ser-Gly-Gly-Gly-Gly-Gly-Gly
(SGGGGGG: SEQ ID NO: 19)
(Gly-Gly-Gly-Gly-Ser (GGGGS: SEQ ID NO: 6))n
(Ser-Gly-Gly-Gly-Gly (SGGGG: SEQ ID NO: 11))n

[wherein n is an integer of 1 or greater] It should be noted that the length and sequence for peptide liners can be suitably selected by those skilled in the art depending on the purposes.

In one further preferred embodiment, the cytosol-penetrating antigen-binding molecules of the present disclosure may be of any one of molecular forms 1 to 8, described below.

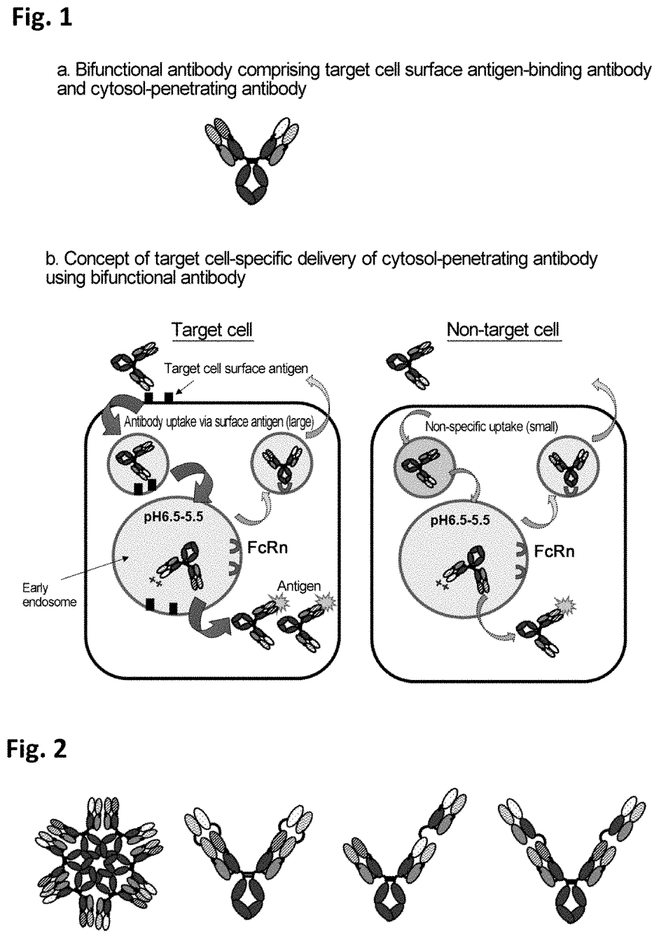

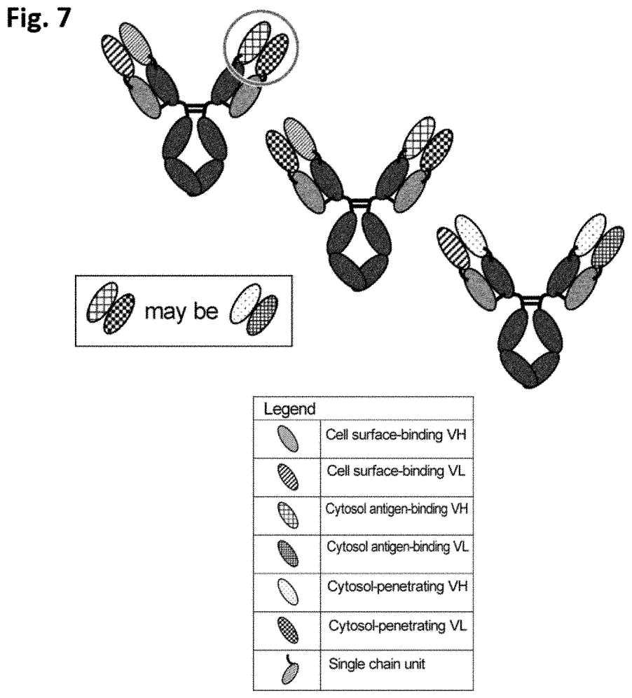

### 1. Cytosol-Penetrating Antigen-Binding Molecules Comprising a First and a Second Fab Regions (FIG. 1 and FIG. 7)

In one preferred embodiment of the present disclosure, the cytosol-penetrating antigen-binding molecules comprise a first and a second Fab regions.

In a certain embodiment, (a) the first Fab region binds specifically to a cell surface antigen; (b) the second Fab region comprises (i) a pair of a heavy chain variable region (VH) binding specifically to a cytosolic antigen and a light chain variable region (VL) having cytosol-penetrating ability or (ii) a pair of a heavy chain variable region (VH) having cytosol-penetrating ability and a light chain variable region (VL) binding specifically to a cytosolic antigen.

In another certain embodiment, (a) the first Fab region comprises a pair of a heavy chain variable region (VH) binding specifically to a cell surface antigen and a light chain variable region (VL) having cytosol-penetrating ability, (b) the second Fab region comprises a pair of a heavy chain variable region (VH) binding specifically to a cytosolic antigen and a light chain variable region (VL) having cytosol-penetrating ability.

In yet another certain embodiment, (a) the first Fab region comprises a pair of a heavy chain variable region (VH) having cytosol-penetrating ability and a light chain variable region (VL) binding specifically to a cell surface antigen, (b) the second Fab region comprises a pair of a heavy chain variable region (VH) having cytosol-penetrating ability and a light chain variable region (VL) binding specifically to a cytosolic antigen.

The cytosol-penetrating antigen-binding molecules in these embodiments may further comprise an Fc region, and the Fc region may comprise a modification that enhances association of a first Fc subunit and a second Fc subunit.

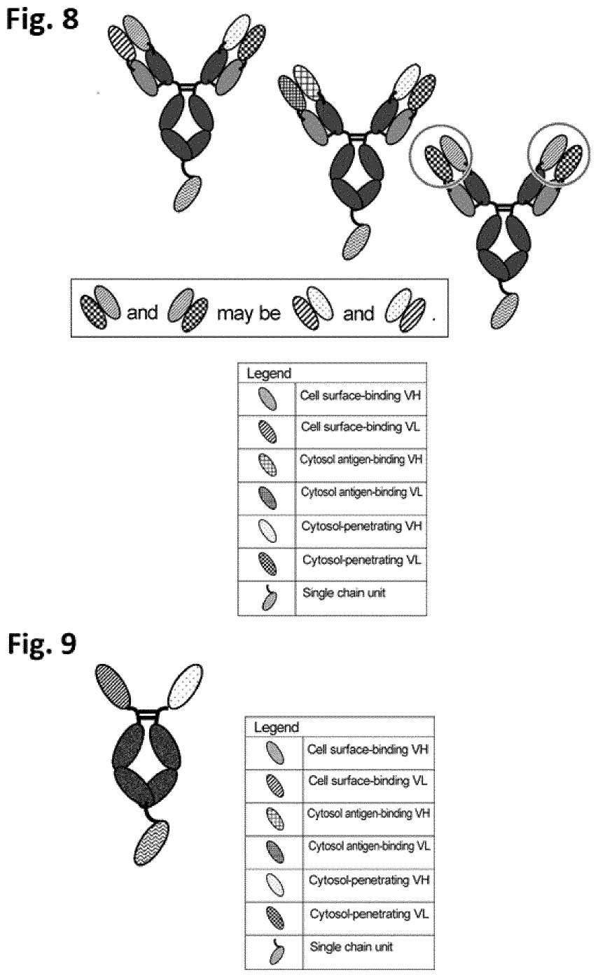

### 2. Cytosol-Penetrating Antigen-Binding Molecules Comprising a First and a Second Fab Regions and a Single-Chain Unit (FIG. 8)

In one preferred embodiment of the present disclosure, the cytosol-penetrating antigen-binding molecules comprise a first and a second Fab regions and a single-chain unit.

In a certain embodiment, (a) the first Fab region binds specifically to a cell surface antigen; (b) the second Fab region has cytosol-penetrating ability; (c) the single-chain unit binds specifically to a cytosolic antigen.

In another certain embodiment, (a) the first Fab region binds specifically to a cytosolic antigen; (b) the second Fab region has cytosol-penetrating ability; (c) the single-chain unit binds specifically to a cell surface antigen.

In yet another certain embodiment, (a) the first Fab region binds specifically to a cell surface antigen; (b) the second Fab region binds specifically to a cytosolic antigen; (c) the single-chain unit has cytosol-penetrating ability.

In yet another certain embodiment, (a) the first and the second Fab regions comprise (i) a pair of a heavy chain variable region (VH) binding specifically to a cell surface antigen and a light chain variable region (VL) having cytosol-penetrating ability or (ii) a pair of a heavy chain variable region (VH) having cytosol-penetrating ability and a light chain variable region (VL) binding specifically to a cell surface antigen; (b) the single-chain unit binds specifically to a cytosolic antigen.

In a certain embodiment, the single-chain unit is fused to (i) the N terminus of the heavy chain variable region (VH) of the first Fab region and/or the N terminus of the heavy chain variable region (VH) of the second Fab region; (ii) the N terminus of the light chain variable region (VL) of the first Fab region and/or the N terminus of the light chain variable region of the second Fab region; or (iii) the C terminus of the light chain constant region (CL) of the first Fab region and/or the C terminus of the light chain constant region (CL) of the second Fab region.

The cytosol-penetrating antigen-binding molecules in these embodiments may further comprise an Fc region which comprises a first Fc subunit and a second Fc subunit, and the single-chain unit may be fused to (i) the C terminus of the first Fc subunit or (ii) the C terminus of the second Fc subunit. The first Fc subunit and the second Fc subunit may be heavy chain constant region CH2 and CH3 domains of an IgG antibody, and may comprise a modification that enhances association of the first Fc subunit and the second Fc subunit.

The single-chain unit comprised in the cytosol-penetrating antigen-binding molecules in these embodiments may be a single-domain antibody variable region or a single-chain antibody (scFv). Examples of the single-domain antibody variable region include, but are not limited to, a heavy-chain antibody variable region (VHH), a heavy chain variable region (VH), a light chain variable region (VL), and a variable region of an immunoglobulin new antigen receptor (VNAR).

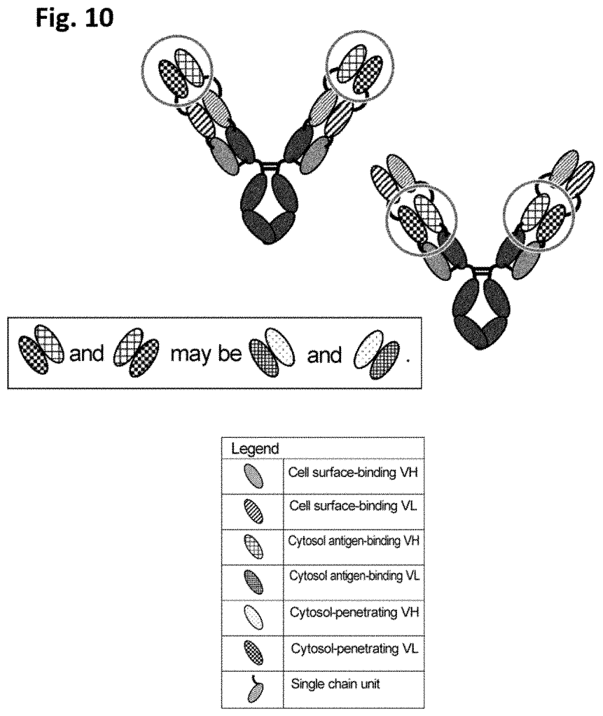

### 3. Cytosol-Penetrating Antigen-Binding Molecules Comprising a First and a Second Single-Domain Antibody Variable Regions, and a Single-Chain Unit (FIG. 9)

In one preferred embodiment of the present disclosure, the cytosol-penetrating antigen-binding molecules comprise a first and a second single domain-antibody variable regions and a single-chain unit.

In a certain embodiment, (a) the first single-domain antibody variable region binds specifically to a cell surface antigen; (b) the second single-domain antibody variable region has cytosol-penetrating ability; (c) the single-chain unit binds specifically to a cytosolic antigen. In a certain embodiment, the single-chain unit is fused to the N terminus of the first single-domain antibody variable region and/or the N terminus of the second single-domain antibody variable region.

The cytosol-penetrating antigen-binding molecules in these embodiments may further comprise an Fc region which comprises a first Fc subunit and a second Fc subunit, and the single-chain unit may be fused to (i) the C terminus of the first Fc subunit or (ii) the C terminus of the second Fc subunit. The first Fc subunit and the second Fc subunit may be heavy chain constant region CH2 and CH3 domains of an IgG antibody, and may comprise a modification that enhances association of the first Fc subunit and the second Fc subunit.

The single-chain unit comprised in the cytosol-penetrating antigen-binding molecules in these embodiments may be a single-domain antibody variable region or a single-chain antibody (scFv). Examples of the single-domain antibody variable region include, but are not limited to, a heavy-chain antibody variable region (VHH), a heavy chain variable region (VH), a light chain variable region (VL), and a variable region of an immunoglobulin new antigen receptor (VNAR).

### 4. Cytosol-Penetrating Antigen-Binding Molecules Having a Molecular Form of DVD-Ig (Registered Trademark) (FIG. 10)

In one preferred embodiment, the cytosol-penetrating antigen-binding molecules of the present disclosure comprise a first and a second polypeptide chains and have the molecular form called DVD-Ig (registered trademark).

In a certain embodiment,
(a) the first polypeptide chain comprises a first VD1-(X1)n-VD2-C-(X2)n, in which VD1 is a first heavy chain variable region binding specifically to a cytosolic antigen, VD2 is a second heavy chain variable region binding specifically to a cell surface antigen, C is a heavy chain constant region CH1, X1 is a linker other than CH1, X2 is an Fc region, and n is 0 or 1;
(b) the second polypeptide chain comprises a second VD1-(X1)n-VD2-C-(X2)n, in which VD1 is a first light chain variable region binding specifically to a cytosolic antigen, VD2 is a second light chain variable region binding specifically to a cell surface antigen, C is a light chain constant region CL, X1 is a linker other than CL, X2 does not comprise an Fc region, and n is 0 or 1.

In another certain embodiment, (a) the first polypeptide chain comprises a first VD1-(X1)n-VD2-C-(X2)n, in which VD1 is a first heavy chain variable region having cytosol-penetrating ability, VD2 is a second heavy chain variable region binding specifically to a cell surface antigen, C is a heavy chain constant region CH1, X1 is a linker other than CH1, X2 is an Fc region, and n is 0 or 1; (b) the second polypeptide chain comprises a second VD1-(X1)n-VD2-C-(X2)n, in which VD1 is a first light chain variable region having cytosol-penetrating ability, VD2 is a second light chain variable region binding specifically to a cell surface antigen, C is a light chain constant region CL, X1 is a linker other than CL, X2 does not comprise an Fc region, and n is 0 or 1.

In yet another certain embodiment, (a) the first polypeptide chain comprises a first VD1-(X1)n-VD2-C-(X2)n, in which VD1 is a first heavy chain variable region binding specifically to a cell surface antigen, VD2 is a second heavy chain variable region binding specifically to a cytosolic antigen, C is a heavy chain constant region CH1, X1 is a linker other than CH1, X2 is an Fc region, and n is 0 or 1; (b) the second polypeptide chain comprises a second VD1-(X1)n-VD2-C-(X2)n, in which VD1 is a first light chain variable region binding specifically to a cell surface antigen, VD2 is a second light chain variable region having cytosol-penetrating ability, C is a light chain constant region CL, X1 is a linker other than CL, X2 does not comprise an Fc region, and n is 0 or 1.

In yet another certain embodiment, (a) the first polypeptide chain comprises a first VD1-(X1)n-VD2-C-(X2)n, in which VD1 is a first heavy chain variable region binding specifically to a cell surface antigen, VD2 is a second heavy chain variable region having cytosol-penetrating ability, C is a heavy chain constant region CH1, X1 is a linker other than CH1, X2 is an Fc region, and n is 0 or 1; (b) the second polypeptide chain comprises a second VD1-(X1)n-VD2-C-(X2)n, in which VD1 is a first light chain variable region binding specifically to a cell surface antigen, VD2 is a second light chain variable region binding specifically to a cytosolic antigen, C is a light chain constant region CL, X1 is a linker other than CL, X2 does not comprise an Fc region, and n is 0 or 1.

Examples of linkers in these embodiments include, but are not limited to, ASTKGP (SEQ ID NO: 20), ASTKGPSVFPLAP (SEQ ID NO: 21), GGGGSG (SEQ ID NO: 22), GGGGSGGGGS (SEQ ID NO: 23), GGGGSGGGGSGGGG (SEQ ID NO: 24), etc. as examples for a linker between the heavy chain variable regions VD1 and VD2; RTVAAP (SEQ ID NO: 25), RTVAAPSVFIFPP (SEQ ID NO: 26), GGSGG (SEQ ID NO: 8), GGSGGGGSG (SEQ ID NO: 27), GGSGGGGSGGGGS (SEQ ID NO: 28), etc. as examples for a linker between the light chain variable regions VD1 and VD2 in the case that the light chain is a kappa chain; GQPKAAP (SEQ ID NO: 29), GQPKAAPSVTLFPP (SEQ ID NO: 30), GGSGG (SEQ ID NO: 8), GGSGGGGSG (SEQ ID NO: 27), GGSGGGGSGGGGS (SEQ ID NO: 28), etc. as examples for a linker between the light chain variable regions VD1 and VD2 in the case that the light chain is a lambda chain. It should be noted that the length and sequence for peptide linkers can be suitably selected by those skilled in the art depending on the purposes.

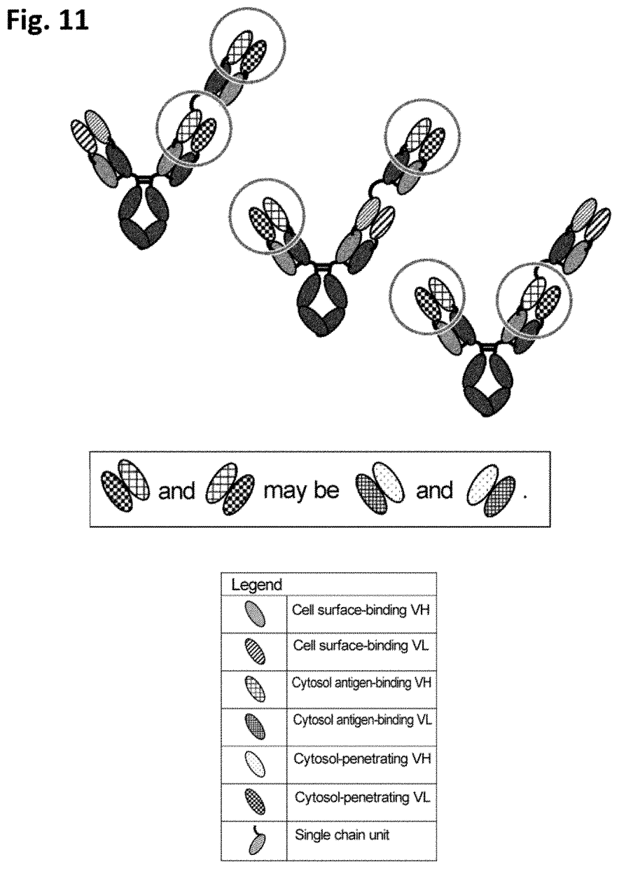

### 5. Cytosol-Penetrating Antigen-Binding Molecules Comprising a First, a Second, and a Third Fab Regions (FIG. 11)

In one preferred embodiment, the cytosol-penetrating antigen-binding molecules of the present disclosure comprise a first, a second, and a third Fab regions.

In a certain embodiment, (a) the first Fab region binds specifically to a cell surface antigen; (b) the second and the third Fab regions comprise (i) a pair of a heavy chain variable region binding specifically to a cytosolic antigen and a light chain variable region having cytosol-penetrating ability or (ii) a pair of a heavy chain variable region having cytosol-penetrating ability and a light chain variable region binding specifically to a cytosolic antigen; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In another certain embodiment, (a) the first and the third Fab regions comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen; (b) the second Fab region binds specifically to a cell surface antigen; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In yet another certain embodiment, (a) the first Fab region and the second Fab region comprise (i) a pair of a heavy chain variable region binding specifically to a cytosolic antigen and a light chain variable region having cytosol-penetrating ability or (ii) a pair of a heavy chain variable region having cytosol-penetrating ability and a light chain variable region binding specifically to a cytosolic antigen; (b) the third Fab region binds specifically to a cell surface antigen; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In a certain embodiment, the first Fab region and/or the second Fab region comprises any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the first Fab region and the second Fab region do not comprise the same exchange.

In these embodiments, the first Fc subunit and the second Fc subunit may be heavy chain constant region CH2 and CH3 domains of an IgG antibody, and may comprise a modification that enhances association of the first Fc subunit and the second Fc subunit.

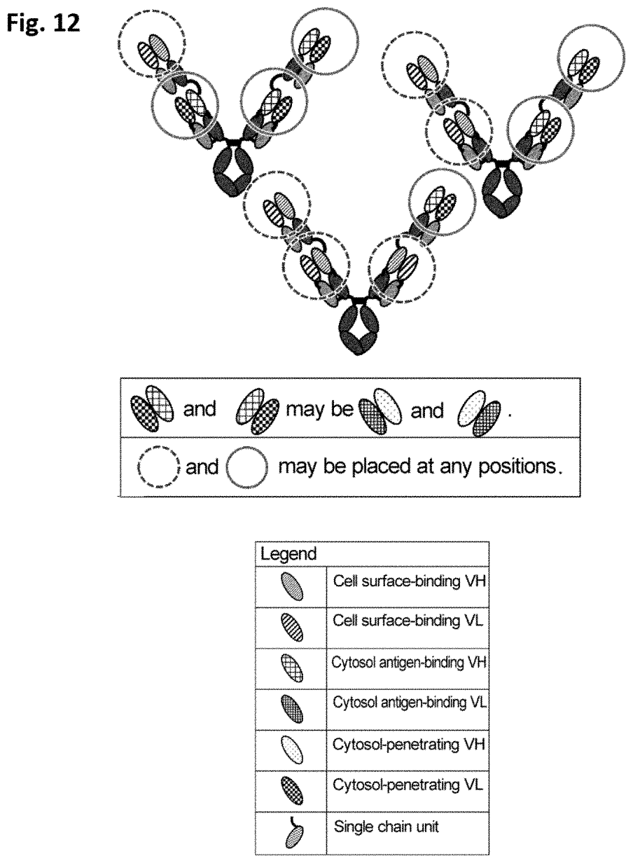

### 6. Cytosol-Penetrating Antigen-Binding Molecules Comprising a First, a Second, a Third, and a Fourth Fab Regions (FIG. 12)

In one preferred embodiment, the cytosol-penetrating antigen-binding molecules of the present disclosure comprise a first, a second, a third, and a fourth Fab regions.

In a certain embodiment,
(a) one Fab region selected from the first, the second, the third, and the fourth Fab regions binds specifically to a cell-surface antigen; (b) three Fab regions other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In another certain embodiment, (a) two Fab regions selected from the first, the second, the third, and the fourth Fab regions bind specifically to a cell surface antigen; (b) two Fab regions other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In yet another certain embodiment, (a) three Fab regions selected from the first, the second, the third, and the fourth Fab regions bind specifically to a cell surface antigen; (b) one Fab region other than (a) comprise (i) a pair of a heavy chain variable region binding specifically to a cytosolic antigen and a light chain variable region having cytosol-penetrating ability, or (ii) a pair of a heavy chain variable region having cytosol-penetrating ability and a light chain variable region binding specifically to a cytosolic antigen; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In a certain embodiment, the first Fab region and/or the second Fab region comprise any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the first Fab region and the second Fab region do not comprise the same exchange.

In another certain embodiment, the third Fab region and/or the fourth Fab region comprise any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the third Fab region and the fourth Fab region do not comprise the same exchange.

In these embodiments, the first Fc subunit and the second Fc subunit may be heavy chain constant region CH2 and CH3 domains of an IgG antibody, and may comprise a modification that enhances association of the first Fc subunit and the second Fc subunit.

7. Cytosol-Penetrating Antigen-Binding Molecules Comprising an Altered Fc Region that Enhances Formation of a Multimer (FIG. 2)

In one preferred embodiment, the cytosol-penetrating antigen-binding molecule of the present disclosure comprises a region having cytosol-penetrating ability and an Fc region, the Fc region comprising one or more amino acid alterations for enhancing formation of a multimer of the antigen-binding molecule, the antigen-binding molecule having an elevated cytosol-penetrating ability as compared to a cytosol-penetrating antigen-binding molecule comprising a parent Fc region which does not comprise the one or more amino acid alterations. The cytosol-penetrating antigen-binding molecule may further comprise a cell surface antigen-binding domain and a cytosolic antigen-binding domain.

In a certain embodiment, the cytosol-penetrating antigen-binding molecule comprises a first and a second Fab regions and an Fc region, and (a) the first Fab region binds specifically to a cell surface antigen; (b) the second Fab region comprises (i) a pair of a heavy chain variable region (VH) binding specifically to a cytosolic antigen and a light chain variable region (VL) having cytosol-penetrating ability, or (ii) a pair of a heavy chain variable region (VH) having cytosol-penetrating ability and a light chain variable region (VL) binding specifically to a cytosolic antigen; (c) the Fc region comprises one or more amino acid alterations for enhancing formation of a multimer of the antigen-binding molecule. The cytosol-penetrating antigen-binding molecules comprising such an altered Fc region have an elevated cytosol-penetrating ability as compared to a cytosol-penetrating antigen-binding molecule comprising a parent Fc region which does not comprise the one or more amino acid alterations, and can allow formation of a multimeric complex of the cytosol-penetrating antigen-binding molecule that comprises a plurality of the cytosol-penetrating antigen-binding molecules.

The multimer may be a dimer, a trimer, a tetramer, a pentamer, or a hexamer. The amino acid alterations may be the combination E345R/E430G/S440Y or the combination T437R/K248E (the numbers represent positions of substitutions according to EU numbering).

In these embodiments, the first Fc subunit and the second Fc subunit may be heavy chain constant region CH2 and CH3 domains of an IgG antibody, and may comprise a modification that enhances association of the first Fc subunit and the second Fc subunit.

### 8. Cytosol-Penetrating Antigen-Binding Molecules Conjugated to Heterologous Moieties

In one aspect, the antigen-binding molecule of the present disclosure is a cytosol-penetrating antigen-binding molecule, which is conjugated to a heterologous moiety. In one embodiment, the cytosol-penetrating antigen-binding molecule of the present disclosure comprises a cell surface antigen-binding domain and a cytosol-penetrating domain, and does not comprise a cytosolic antigen-binding domain. In one preferred embodiment, the cytosol-penetrating antigen-binding molecule of the present disclosure does not bind to antigens that are expressed on the surface of a cell and different from the aforementioned cell surface antigen. In one preferred embodiment, the cytosol-penetrating domain and/or the heterologous moiety (i) do not bind to antigens that are expressed on the surface of a cell and different from the aforementioned cell surface antigen; or (ii) do not bind to any antigens expressed on the surface of a cell.

In one aspect, as described above, the present inventors arrived at the idea of using a monovalent cytosol-penetrating domain to suppress nonspecific uptake, adding a target cell surface-binding domain to improve target specificity, and, at the same time, creating conditions where the interaction of antibodies and endosome membrane occurs in a multivalent fashion, in order to keep or improve cytosol-penetrating ability.

In one embodiment, the cytosol-penetrating antigen-binding molecules of the present disclosure comprise a cell surface antigen-binding domain, and a cytosol-penetrating domain. Therefore, in one embodiment, for the cytosol-penetrating antigen-binding molecules of the present disclosure it is expected that it binds to a cell surface antigen on a target cell and thereby undergoes endocytosis in a target cell-specific manner, and then it translocates itself from the endosomes into the cytosol, and thereby delivers the heterologous moieties into the cytosol.

In one embodiment, the cytosol-penetrating antigen-binding molecules of the present disclosure are more selectively delivered into the cytosol of target cells as compared to the already existing cytosol-penetrating antibodies, so that the cytosol-penetrating antigen-binding molecules of the present disclosure can deliver the heterologous moieties more selectively into the cytosol of target cells. In one embodiment, a larger amount of the heterologous moieties conjugated to the cytosol-penetrating antigen-binding molecules is delivered to the cytosol of a target cell when a fixed amount of the cytosol-penetrating antigen-binding molecules of the present disclosure is administered or is contacted with a subject, as compared to the case of the same amount of the heterologous moieties conjugated to the already existing cytosol-penetrating antibodies. In one embodiment, the heterologous moieties conjugated to the cytosol-penetrating antigen-binding molecules of the present disclosure are delivered specifically into the cytosol of a target cell, while substantially not delivered to cells that do not express the cell surface antigen to which the antigen-binding molecules specifically bind.

In one embodiment, when the cytosol-penetrating antigen-binding molecules of the present disclosure are used as pharmaceuticals, the heterologous moieties conjugated to the cytosol-penetrating antigen-binding molecules exert stronger drug efficacy and/or cause lesser side effects, as compared to the heterologous moieties conjugated to the already existing cytosol-penetrating antibodies. In one embodiment, pharmaceutical compositions comprising the heterologous moieties conjugated to the cytosol-penetrating antigen-binding molecules can exert drug efficacy with smaller amount of administration and/or lesser number of administrations as compared to pharmaceutical compositions comprising the heterologous moieties conjugated to the already existing cytosol-penetrating antibodies.

In one embodiment, the cytosol-penetrating antigen-binding molecule comprises a first and a second Fab regions, wherein (a) the first Fab region binds specifically to a cell surface antigen, and (b) the second Fab region has cytosol-penetrating ability, and wherein the antigen-binding molecule is conjugated to a heterologous moiety. In a preferred embodiment, the heterologous moiety is conjugated to the C-terminus (preferably, the biotinylated C-terminus) of the L chains of the antigen-binding molecule.

In one embodiment, the heterologous moiety conjugated to the cytosol-penetrating antigen-binding molecule is a peptide, nucleic acid, chemotherapeutic agent or drug, growth inhibitory agent, toxin (e.g., protein toxin, enzymatically active toxin of bacterial, fungal, plant, or animal origin, or a fragment thereof), or radioactive isotope. In one embodiment, the heterologous moiety is a peptide.

### B. Antibodies

In a further aspect of the present disclosure, the antigen-binding molecules according to any of the above-described embodiments are antibodies and, in one preferred embodiment, monoclonal antibodies including chimeric antibodies, humanized antibodies, or human antibodies. In one embodiment, the antigen-binding molecules are antibody fragments such as Fv, Fab, Fab′, scFv, diabodies, or F(ab′)2 fragments. In another embodiment, the antibodies are full-length antibodies, for example, intact IgG1 antibodies or full-length antibodies of other antibody classes or isotypes defined herein.

In a further aspect, the antibodies according to any of the above-described embodiments may incorporate any characteristics described below in items 1 to 7, alone or in combination.

### 1. Binding Activity and Affinity of Antibodies

In certain embodiments, binding activity or affinity of antibodies provided herein is a dissociation constant (KD) of 1 micro M or less, 100 nM or less, 10 nM or less, 1 nM or less, 0.1 nM or less, 0.01 nM or less, or 0.001 nM or less (for example, 10-8 M or less, for example, 10-8 M to 10-13 M, and for example, 10-9 M to 10-13 M).

### 2. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

### 4. Human Antibodies

In certain embodiments, an antibody provided herein is a human antibody.

Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

### 5. Library-Derived Antibodies

Antibodies of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

### 6. Multifunctional Antibodies

In certain embodiments, the antibodies provided herein are multifunctional antibodies. Multifunctional antibodies are monoclonal antibodies having functions different from one another at least two different sites. For example, multifunctional antibodies are monoclonal antibodies having two functions, that is, (i) binding specificity and (ii) a function other than binding specificity. Examples of functions other than binding specificity include cytosol-penetrating ability. In one embodiment, the multifunctional antibodies are bifunctional antibodies (having two functions which are antigen-binding specificity and cytosol-penetrating ability). In one embodiment, the multifunctional antibodies are multispecific antibodies (e.g., bispecific antibodies). Multispecific antibodies are monoclonal antibodies having binding specificities at least two different sites. Bispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

It will be understood that, for making multifunctional antibodies, techniques similar to the above-described techniques for making multispecific antibodies can be used.

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACT1TM non-radioactive cytotoxicity assay for flow cytometry (CellTechnology, Inc. Mountain View, Calif.; and CytoTox 96 (registered trademark) non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### C. Recombinant Methods and Compositions

Antibodies may be produced using recombinant methods and compositions, e.g., as described in U.S. Pat. No. 4,816,567. In one embodiment, isolated nucleic acid encoding an antigen-binding molecule described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp2/0 cell). In one embodiment, a method of making an antigen-binding molecule of the present disclosure is provided, wherein the method comprises culturing a host cell comprising a nucleic acid encoding the antigen-binding molecule, as provided above, under conditions suitable for expression of the antigen-binding molecule, and optionally recovering the antigen-binding molecule from the host cell (or host cell culture medium).

For recombinant production of an antigen-binding molecule of the present disclosure, nucleic acid encoding an antigen-binding molecule, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When the antigen-binding molecule of the present disclosure is an antibody, suitable host cells for cloning or expression of vectors that encode the antibody include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### D. Assays

Antigen-binding molecules provided herein may be identified, screened for, or characterized for their physical/chemical properties and/or biological activities by various assays known in the art.

### 1. Measurement of Binding Activity and Affinity

In one embodiment, for measuring binding activity or affinity of an antibody, ligand-capturing methods using BIACORE (registered trademark) T200 or BIACORE (registered trademark) 4000 (GE Healthcare, Uppsala, Sweden), the measurement by which relies upon the principle of surface plasmon resonance analysis, are used. BIACORE (registered trademark) Control Software is used for operation of devices. In one embodiment, amine-coupling kit (GE Healthcare, Uppsala, Sweden) is used according to the manufacturer's instructions to immobilize a molecule for ligand capturing, for example, an anti-tag antibody, an anti-IgG antibody, protein A, etc., onto a sensor chip (GE Healthcare, Uppsala, Sweden) coated with carboxymethyldextran. The ligand-capturing molecule is diluted with a 10 mM sodium acetate solution at an appropriate pH and is injected at an appropriate flow rate and for an appropriate injection time. Binding activity is measured using a 0.05% polysorbate 20 (otherwise known as Tween (registered trademark)-20)-containing buffer as a measurement buffer, at a flow rate of 10-30 micro L/minute, and at a measurement temperature of preferably at 25 degrees C. or 37 degrees C. For the measurement carried out by allowing the ligand-capturing molecule to capture an antibody as a ligand, an antibody is injected to let a target amount of the antibody captured, and then a serial dilution of an antigen and/or an Fc receptor (analyte) prepared using the measurement buffer is injected. For the measurement carried out by allowing the ligand-capturing molecule to capture an antigen and/or an Fc receptor as a ligand, an antigen and/or an Fc receptor is injected to let a target amount thereof captured, and then a serial dilution of an antibody (analyte) prepared using the measurement buffer is injected.

In one embodiment, the measurement results are analyzed using BIACORE (registered trademark) Evaluation Software. For example, kinetics parameter analysis is carried out by fitting sensorgrams of association and dissociation at the same time using a 1:1 binding model, and an association rate (kon or ka), a dissociation rate (koff or kd), and an equilibrium dissociation constant (1(D) may be calculated. For the case of weak binding activity, in particular, for the cases where dissociation is fast and kinetics parameters are difficult to calculate, the Steady state model may be used to calculate the equilibrium dissociation constant (KD). As additional parameters concerning binding activity, “amount of bound analyte per unit ligand amount” may be calculated by dividing the amount of bound analyte (RU) at a specific concentration by the amount of captured ligand.

### 2. Measurement of Antigen-Binding Molecules in the Cytosol

In one embodiment, an antigen-binding molecule in the cytosol is measured at an arbitrary time point after letting the antigen-binding molecule contact with a cell. The contact between the antigen-binding molecule and cell is achieved by any methods including incubation. When the antigen-binding molecule is contacted with a cell by incubation, the duration of the incubation and the period of time after the incubation and before the measurement of the antigen-binding molecule in the cytosol are arbitrarily selected. For example, in the case that an antigen-binding molecule present within the cytosol is measured only once after contacting the antigen-binding molecule with a cell, the antigen-binding molecule and the cell are incubated for 1 hour, 2 hours, 3 hours, 4 hours, 5 hours, or 6 hours, and after that the culture medium containing the antigen-binding molecule is removed and the antigen-binding molecule in the cytosol may be measured immediately. For example, in the case that the antigen-binding molecule present within the cytosol is measured for two or more times after contacting the antigen-binding molecule with a cell, the antigen-binding molecule and the cell are incubated for 1 hour, 2 hours, 3 hours, 4 hours, 5 hours, or 6 hours, and after that the culture medium containing the antigen-binding molecule is removed, and further incubation is carried out using a new culture medium free of the antigen-binding molecule for 0 hour, 0.25 hour, 0.5 hour, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, and/or 16 hours, and then, the antigen-binding molecule in the cytosol may be measured. By detecting an antigen-binding molecule in the cytosol at an arbitrary time point after letting the antigen-binding molecule contact with a cell, the cytosol-penetrating ability of the antigen-binding molecule can also be detected or assessed. Therefore it is understood that the present disclosure also provides a method for detecting the cytosol-penetrating ability of the antigen binding molecule.

### a) BirA Assay

In one embodiment, the amount of an antigen-binding molecule present in the cytosol at an arbitrary time point can be evaluated in the BirA assay by detecting a fusion of a biotinylated Avi tag and the antigen-binding molecule by using a labeled biotin-binding protein. The biotin-labeled Avi tag is measured using an avidin-peroxidase conjugate and an appropriate substrate. For example, when the biotinylated Avi tag is detected using streptavidin-HRP, the amount of the antigen-binding molecule present in the cytosol can be represented by the HRP luminescence signal intensity. The BirA assay is a widely-known method among those skilled in the art and is described, for example, in W. P. R. Verdurmen et al., Journal of Controlled Release 200 (2015) 13-22 and in the Examples of the present disclosure. The conditions used for the assays to determine the amount of the antigen-binding molecule present in the cytosol may be suitably selected by those skilled in the art, and thus are not particularly limited. The fusion of the biotinylated Avi tag and the antigen-binding molecule in the cytosol may be further labeled with another labeling substance that can be detected or measured. Specifically, radioactive labels and fluorescent labels are known. The detection of labeled biotin-binding protein in the BirA assay (e.g., detection of HRP luminescence signal for the case of streptavidin-HRP) can be suitably carried out by methods known among those skilled in the art. Specifically, western blotting or capillary immuno assay (ProteinSimple, Inc.), etc. are known.

### b) Imaging by Fluorescent Microscopy

In another embodiment, the amount of the antigen-binding molecule present in the cytosol at an arbitrary time point can be evaluated by detecting using a labeled protein that binds to the antigen-binding molecule. For example, when the antigen-biding molecule comprise an IgG antibody, the antigen-binding molecule present in the cytosol can be detected using a labeled anti-IgG antibody. Regarding the label, radioactive labels and fluorescent labels are known, for example. The detection of the labeled antigen-binding molecule can be suitably carried out by methods known among those skilled in the art. For example, the labeled antigen-binding molecule can be detected by imaging the fluorescent signal using a fluorescent microscope, as described herein and in the Examples of the present disclosure.

### c) Split-GFP

In another embodiment, the amount of the antigen-binding molecule present in the cytosol at an arbitrary time point can be evaluated by detecting GFP fluorescent signal in the split-GFP complementation system. Specific methods are known to those skilled in the art and are described, for example, in WO 2016/013870. Specifically, when green fluorescent protein (GFP) is split into fragments 1-10 and fragment 11, GFP's feature of exhibiting fluorescence is removed, but the fluorescence-exhibiting feature can be recovered if the two fragments are brought close and bind to each other (Cabantous et al., 2005). To make use of this, the fragment of GFP1-10 is expressed in the cytosol and the fragment of GFP11 is fused to an arbitrary site on an antigen-binding molecule. If GFP fluorescence is observed in this method, it is indicated that the two GFP fragments bind together, i.e., the antigen-binding molecule is present in the cytosol.

### d) Improved Split Protein System

In another embodiment, the amount of the antigen-binding molecule present in the cytosol at an arbitrary time point can be evaluated by detecting a luminescent and/or fluorescent signal in the split protein system. In one embodiment, the split protein system comprises (i) a peptide comprising a mitochondrial outer membrane protein and a first fragment of a luminescent protein, and (ii) a second fragment of the luminescent protein, wherein the first fragment and the second fragment of the luminescent protein together contain the full complement of the luminescent protein. In the split protein system, the first fragment and the second fragment of the luminescent protein does not have the feature of exhibiting luminescence, but the luminescence-exhibiting feature is recovered when the first fragment of the luminescent protein comprised in the peptide binds to the second fragment of the luminescent protein. Methods to evaluate cytosol penetrating ability using Nanoluc complementation (Schaub et al., Cancer Res. (2015) 75(23):5023, Dixon et al., ACS Chem Biol. (2016) 11(2):400) were reported, but the background luminescence and/or fluorescent signal of the methods were still high. In view of these, in one embodiment, the present inventors arrived at the idea of using a mitochondrial outer membrane protein in the split protein system in order to reduce the background signal.

In one embodiment, the split luminescent protein is a split NanoLuc (registered trademark). NanoLuc is a small 19 kDa luciferase enzyme engineered from a deep sea luminous shrimp. The split NanoLuc assay utilizes NanoLuc which is cleaved into two sub-units—a small 1.3 kDa Small BiT (SmBiT) (SEQ ID NO: 63) and a larger 18 kDa Large BiT (LgBiT) (SEQ ID NO: 62). Complementation of the LgBiT and SmBiT is observed as luminescence from, enzymatic reaction on substrate, in live cells by use of the NanoLuc Live Cell Assay System (Promega). In the split NanoLuc assay, HeLa cells were genetically modified to stably express NanoLuc LgBiT intracellularly. Usually, the NanoLuc LgBiT can be expressed as free cytosolic protein. However, in one embodiment, when the NanoLuc LgBiT is tethered to an intracellular structure within the cytosol, for instance, the mitochondrial outer membrane. The present inventors surprisingly discovered that the tethering of the NanoLuc LgBiT to the mitochondrial outer membrane resulted in significantly lower background signal and better assay sensitivity compared to when NanoLuc LgBiT was expressed as free cytosolic protein. On the other hand, the SmBiT conjugate can be fused to an arbitrary site on an antigen-binding molecule. Preferably, the antigen-binding molecule is an antibody and the second fragment of a luminescent protein is fused to the C-terminus of the heavy chains of the antibody. The antigen-binding molecule-SmBiT conjugates penetrate into the cytosol and thereby complement the NanoLuc LgBiT luciferase enzyme, which can be detected by enhancement of luminescence.

In one example, the present disclosure provides an improved split protein system, which comprises (i) a peptide comprising a mitochondrial outer membrane protein and a first fragment of a luminescent protein, and (ii) a second fragment of the luminescent protein, wherein the first fragment and the second fragment of the luminescent protein together contain the full complement of the luminescent protein. In another example, the present disclosure provides a peptide used in the improved split protein system, wherein the peptide comprising a mitochondrial outer membrane protein and a first fragment of a luminescent protein, and wherein the first fragment and a second fragment of the luminescent protein together contain the full complement of the luminescent protein.

An example of the first fragment of the luminescent protein is NanoLuc Large BiT (LgBiT). An example of the second fragment of the luminescent protein is NanoLuc SmBiT (SmBiT). In a further embodiment, the peptide comprising the mitochondrial outer membrane protein and the first fragment of the luminescent protein further comprises a green fluorescent protein (GFP) or its variant. Preferably, the peptide comprising the mitochondrial outer membrane protein, the first fragment of the luminescent protein, and a GFP variant comprises an amino acid sequence of SEQ ID NO: 50.

A mitochondrial outer membrane protein used in the improved split protein system can be any protein which is localized on the mitochondrial outer membrane. The mitochondrial outer membrane protein can be, for example, AKAP1, BAK1, FIS1, CYBSB, FISS, FUNDC1, GDAP1, MARC1, MARC2, MFN1, MFN2, MFN3, MIEF1, MIEF2, MID49, MID51, MIGA1, MIGA2, MIRO1, MIRO2, MSTO1, MTX1, MTX2, MTX3, PGAMS, PLD6, RAB32, RMDN3, SYNJ2BP, TOMS, TOM6, TOM7, TOM20, TOM22, TOM34, TOM70, TSPO, and/or UBP30.

In a preferred embodiment, the mitochondrial outer membrane protein is an additional mitochondrial kinase anchoring protein 1 (AKAP1; also known as A-kinase anchoring protein 1). In a preferred embodiment, the mitochondrial outer membrane protein comprises an amino acid sequence of SEQ ID NO: 61. In a preferred embodiment, the peptide comprising the first fragment of the luminescent protein comprises an amino acid sequence of SEQ ID NO: 50 (AKAP1-eGFP-NanoLuc LgBit).

In one embodiment, the present disclosure provides a nucleic acid encoding the peptide comprising the mitochondrial outer membrane protein and the first fragment of the fluorescent protein, a vector comprising said nucleic acid, a cell comprising said nucleic acid or said vector, or a kit comprising said protein, nucleic acid, vector, or cell. In further embodiment, the present disclosure provides a method for detecting a target molecule present in the cytosol, which comprises (i) providing a cell comprising a nucleic acid encoding a peptide comprising a mitochondrial outer membrane protein and a first fragment of a fluorescent protein, (ii) providing a target molecule conjugated with a second fragment of the fluorescent protein, (iii) contacting the cell of (i) and the target molecule of (ii), and (iv) detecting luminescence signal in the cell of (iii), wherein the first fragment and the second fragment of the fluorescent protein together contain the full complement of the fluorescent protein. The method provided herein has lower background luminescence signal compared with a method which is the same except that the peptide of (i) does not comprise the mitochondrial outer membrane protein. In a preferred embodiment, the target molecule is a cytosol-penetrating antigen binding molecule.

In another embodiment, in the improved split protein system, any protein which is localized on any intracellular structures within the cytosol can be used instead of the mitochondrial outer membrane protein. An example of the protein localized on the intracellular structure is a nucleus outer membrane protein, and the nucleus outer membrane protein can be any protein which is localized on the nucleus outer membrane. The nucleus outer membrane protein can be, for example, NUP37, NUP43, NUP96, NUP85, NUP88, NUP107, NUP133, NUP160, NUP214, and/or NUP358.

### E. Methods of Producing an Antigen-Binding Molecule, Methods of Screening for an Antigen-Binding Molecule, and Methods of Imaging a Cytosolic Antigen

### 1. Methods of Producing an Antigen-Binding Molecule

In one aspect, the present disclosure provides methods of producing the antigen-binding molecules of the present disclosure.

In one embodiment, the method of producing comprises:
(a) obtaining an expression vector that comprises a gene encoding the antigen-binding molecule and an operably linked suitable promoter,
(b) introducing the vector into a host cell and culturing the host cell to allow production of the antigen-binding molecule, and
(c) recovering the antigen-binding molecule from the host cell culture.

In another embodiment, the above-described method of production comprises introducing one or more amino acid alterations into an Fc region of a parent cytosol-penetrating antigen-binding molecule, the alterations enhancing formation of multimers of the cytosol-penetrating antigen-binding molecule as compared to the parent cytosol-penetrating antigen-binding molecule, and cytosol-penetrating ability of the cytosol-penetrating antigen-binding molecule being potentiated as compared to the parent cytosol-penetrating antigen-binding molecule.

### 2. Methods of Screening for a Cytosol-Penetrating Antigen-Binding Molecule

In one aspect, the present disclosure provides methods of screening for a cytosol-penetrating antigen-binding molecule.

In one embodiment, the screening method comprises
(a) providing a parent cytosol-penetrating antigen-binding molecule comprising an Fc region,
(b) obtaining a candidate molecule comprising an altered Fc region by introducing one or more amino acid alterations into the Fc region of the parent cytosol-penetrating antigen-binding molecule,
(c) determining whether the candidate molecule forms a multimer,
(d) identifying the candidate molecule as a suitable molecule when the candidate molecule forms more multimers as compared to the parent cytosol-penetrating antigen-binding molecule.

In a certain embodiment, cytosol-penetrating ability of the cytosol-penetrating antigen-binding molecule comprising the altered Fc region is potentiated as compared to the parent cytosol-penetrating antigen-binding molecule.

### 3. Methods of Imaging a Cytosolic Antigen

In another aspect, the present disclosure provides methods of imaging a cytosolic antigen comprising using the antigen-binding molecule of the present disclosure, the antigen-binding molecule produced by the above-described production method, or the cytosol-penetrating antigen-binding molecule identified as a suitable molecule by the above-described screening method.

In another aspect, the present disclosure provides the antigen-binding molecule of the present disclosure, the antigen-binding molecule produced by the above-described production method, or the cytosol-penetrating antigen-binding molecule identified as a suitable molecule by the above-described screening method for use in imaging a cytosolic antigen in a sample cell.

In yet another aspect, the present disclosure provides use of the antigen-binding molecule of the present disclosure, the antigen-binding molecule produced by the above-described production method, or the cytosol-penetrating antigen-binding molecule identified as a suitable molecule by the above-described screening method in the manufacture of an agent for imaging a cytosolic antigen.

In certain embodiments of these aspects, the antigen-binding molecule to be used is labeled.

### F. Immunoconjugates

The present disclosure also provides immunoconjugates comprising an antigen-binding molecule herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

G. Methods of Delivering an Antigen-Binding Molecule Specifically into Cytosol of a Target Cell and Methods of Potentiating Cytosol-Penetrating Ability of a Cytosol-Penetrating Antigen-Binding Molecule

In one aspect, the present disclosure provides methods of delivering an antigen-binding molecule specifically into cytosol of a target cell, in which methods a cell surface antigen to which the antigen-binding molecule binds is an antigen expressed specifically on the target cell. In one embodiment, the methods comprise contacting the antigen-binding molecule with the target cell. In one embodiment, the antigen-binding molecule is a multifunctional antigen-binding molecule comprising a cytosol-penetrating antibody or a fragment of the antibody, an antibody binding to a cell surface antigen or a fragment of the antibody, and/or an antibody binding to a cytosolic antigen or a fragment of the antibody in combination. In a certain embodiment, the above-described method substantially does not deliver the antigen-binding molecule to cells that do not express the cell surface antigen. In a certain embodiment, the antigen-binding molecule binds to a cell surface antigen specific to the target cell, and thereby undergoes internalization specifically by the target cell and transfers into the cytosol. In a certain embodiment, the antigen-binding molecule forms a multimer, and displays high cytosol-penetrating ability.

In another aspect, the present disclosure provides methods of potentiating cytosol-penetrating ability of a cytosol-penetrating antigen-binding molecule as compared to a parent cytosol-penetrating antigen-binding molecule, the methods comprising introducing into an Fc region one or more amino acid alterations for enhancing formation of a multimer, the parent cytosol-penetrating antigen-binding molecule comprising a parent Fc region that does not comprise the one or more amino acid alterations.

In another aspect, the present disclosure provides a method of delivering a heterologous moiety specifically into cytosol of a target cell, wherein the heterologous moiety is conjugated to an antigen-binding molecule, which comprises a cell surface antigen-binding domain and a cytosol-penetrating domain. Preferably, the antigen-binding molecule comprises a first and a second Fab regions, wherein (a) the first Fab region binds specifically to a cell surface antigen, and (b) the second Fab region has cytosol-penetrating ability, and wherein the antigen-binding molecule is conjugated to the heterologous moiety. More preferably, the antigen-biding molecule is a bifunctional antibody and does not comprise a cytosolic antigen-binding domain. In one embodiment, the cell surface antigen to which the antigen-binding molecule binds is an antigen expressed specifically on the target cell. In one embodiment, the method comprises contacting said antigen-binding molecule conjugated to the heterologous moiety with the target cell. In a preferred embodiment, the method comprises delivering the heterologous moiety specifically into cytosol of the target cell. In a preferred embodiment, the method substantially does not deliver the heterologous moiety to cells that do not express the cell surface antigen.

### H. Methods for Knocking-Down a Cytosolic Antigen

In a certain embodiment, the binding molecules of the present disclosure have an elevated ability to remove a cytosolic antigen in a target cell. Therefore, the antigen-binding molecules provided herein are useful for knocking-down, at protein level, the cytosolic antigen in the target cell. The term “knock(ing) down” used herein means that the amount of expression or abundance of a certain protein is reduced. Specifically, when a cytosolic antigen is knocked-down at protein level, the amount of the cytosolic antigen per cell is reduced.

### I. Methods and Compositions for Diagnosis and Detection

In a certain embodiment, the antigen-binding molecules provided herein are useful in detecting the presence of a cytosolic antigen in a target cell in a biological sample. The term “detection/detecting” used herein encompasses quantitative or qualitative detection.

In one embodiment, the antigen-binding molecules for use in the methods of diagnosis or the methods of detection are provided. In a further aspect, methods of detecting the presence of a cytosolic antigen in a target cell in a biological sample are provided. In a certain embodiment, the method comprises allowing an antigen-binding molecule described herein with a biological sample under the conditions permissible for the binding of the antigen-binding molecule to a cytosolic antigen, and detecting if a complex is formed between the antigen-binding molecule and the cytosolic antigen. Such methods may be in vitro methods or in vivo methods. In a different embodiment, the antigen-binding molecules of the present disclosure are useful for the detection of a cytosolic antigen by imaging or such methods.

In certain embodiments, labeled antigen-binding molecules of the present disclosure are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### J. Pharmaceutical Formulations

Pharmaceutical formulations of an antigen-binding molecule as described herein are prepared by mixing such antigen-binding molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antigen-binding molecule, which matrices are in the form of shaped articles, e.g. films, or microcapsules.

The formulations to be used for in vivo administration are generally sterile. Sterility may be readily accomplished, e.g., by filtration through sterile filtration membranes.

### K. Therapeutic Methods and Compositions

In one aspect, the antigen-binding molecules provided herein may be used in therapeutic methods.

In one embodiment, the antigen-binding molecules of the present disclosure for use as pharmaceuticals are provided. In certain embodiments, the antigen-binding molecules of the present disclosure for use in methods of treatment are provided. In one of these embodiments, the method further comprises a step of administering an effective amount of at least one additional therapeutic agent (e.g., such as those described below) to the subject. The “subject” according to any of the above-described embodiments is preferably a human.

In a further aspect, the present disclosure provides use of the antigen-binding molecules of the present disclosure in the manufacture or preparation of medicaments. In one of such embodiments, the method further comprises a step of administering an effective amount of at least one additional therapeutic agent (e.g., such as those described below) to the subject. The “subject” according to any of the above-described embodiments may be a human.

In a further aspect, the present disclosure provides pharmaceutical formulations comprising any of the antigen-binding molecules provided herein, e.g., for use in any of the above therapeutic methods. In one embodiment, a pharmaceutical formulation comprises any of the antigen-binding molecules provided herein and a pharmaceutically acceptable carrier. In another embodiment, a pharmaceutical formulation comprises any of the antigen-binding molecules provided herein and at least one additional therapeutic agent.

An antigen-binding molecule of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antigen-binding molecules of the present disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antigen-binding molecule need not be, but is optionally formulated with one or more agents currently used to prevent or treat the disorder in question. The effective amount of such other agents depends on the amount of antigen-binding molecule present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antigen-binding molecule of the present disclosure (when used alone or in combination with one or more other additional therapeutic agents) will depend on the type of disease to be treated, the type of antibody when the antigen-binding molecule of the present disclosure is an antibody, the severity and course of the disease, whether the antigen-binding molecule is administered for preventive or therapeutic purposes, previous therapy, the patient's clinical history and response to the antigen-binding molecule, and the discretion of the attending physician. The antigen-binding molecule is suitably administered to the patient at one time or over a series of treatments. Depending on the type and severity of the disease, about 1 micro g/kg to 15 mg/kg (e.g. 0.1 mg/kg-10 mg/kg) of antigen-binding molecule can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen-binding molecule would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antigen-binding molecule). An initial higher loading dose, followed by one or more lower doses may be administered. The progress of this therapy is easily monitored by conventional techniques and assays.

It is understood that any of the above formulations or therapeutic methods may be carried out using an immunoconjugate of the present disclosure in place of or in addition to an antigen-binding molecule.

### L. Articles of Manufacture

In another aspect of the present disclosure, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antigen-binding molecule of the present disclosure. The label or package insert indicates that the composition is used for treating the condition of choice. Moreover, the article of manufacture may comprise (a) a first container with a composition contained therein, wherein the composition comprises an antigen-binding molecule of the present disclosure; and (b) a second container with a composition contained therein, wherein the composition comprises a further cytotoxic or otherwise therapeutic agent. The article of manufacture in this embodiment of the present disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the present disclosure in place of or in addition to an antigen-binding molecule.

### EXAMPLES

The following are examples of methods and compositions of the present disclosure. It is understood that various other embodiments may be practiced, given the general description provided above.

Although the foregoing invention has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

### Example 1

### Concept

Drug discovery technologies that deliver an antibody to the cytosol and allow it to act on an antigen in the cytosol are needed. For example, as for cytotransmab, it has been reported that the antibody in which a light chain variable region that exhibits cytosol-penetrating ability and a heavy chain variable region that binds to activated Ras which is an antigen in the cytosol are combined, shows anti-tumor effects by neutralizing activated Ras in the cytosol (Nat Commun. 2017 May 10; 8:15090). Meanwhile, it has been reported that known cytosol-penetrating antibodies exhibit non-specific cytosol-penetrating ability in various cells. For example, since cytotransmab is internalized into a cell via heparin sulfate proteoglycan (HSPG) which is ubiquitously expressed in epithelial cells, its delivery to cancer tissues is expected to be lowered when it is systemically administered. Thus, to enhance the cancer tissue specificity, Orum Therapeutics prepared the RT11-i antibody in which cytotransmab is fused with the RGD10 circular peptide having binding ability to integrin which is specifically expressed in cancer. However, since the RT11-i antibody also has a light chain that has HSPG-dependent cytosol-penetrating ability (Nat Commun. 2017 May 10; 8:15090), it is possible that the target specificity is still insufficient. To allow an antibody pharmaceutical to act more efficiently on a target molecule in cells and prevent side effects, it is essential to achieve target cell-specific cytosol-penetrating ability. Furthermore, to reduce the dosage and number of administration and develop a more convenient antibody pharmaceutical, it is necessary to increase the cytosol-penetrating ability.

As for the mechanism in which the light chain of cytotransmab has gained cytosol-penetrating ability, it is known that the antibody is incorporated into an endosome by HSPG-dependent endocytosis, and it forms a pore by interacting with the endosomal membrane to allow cytosol penetration (J Control Release. 2016 Aug. 10; 235:165-175). To form the pore on the endosomal membrane, it is thought to be important that the membrane is pulled (twisted) by multivalent interaction with molecules on the membrane. Thus, if the HSPG-binding cytosol-penetrating domain is made to be monovalent to reduce endocytosis into non-target cells via HSPG, the cytosol-penetrating ability is expected to be decreased.

To solve the above problems, the inventors considered that the cytosol-penetrating ability can be maintained or enhanced by constructing a bifunctional antibody from a cytosol-penetrating antibody and an antibody that binds to a receptor on the cell membrane (cell surface antigen) to increase the target tissue specificity and maintain the multivalency of the intraendosomal interaction. Meanwhile, a variant (3D8.03 or Tmab4-03) which lacks HSPG-binding ability but maintains the penetration ability from endosome to cytosol has been reported. Thus, it was thought that, if the bifunctional antibody is constructed from the variant and a cell surface-binding antibody, the incorporation into non-target cells via HSPG can be hampered. Furthermore, it was considered that the cell specificity and cytosol-penetrating ability can be enhanced by making the cell surface antigen-binding domain to be multivalent, and the cytosol-penetrating ability can be increased by making the antibody domain that shows cytosol-penetrating ability to be multivalent.

Thus, attempts were made to construct bifunctional and multivalent antibodies from a known cytosol-penetrating antibody and an antibody against a cell surface antigen, and specifically deliver the bifunctional antibodies to the cytosol of target cells in a cell surface antigen-dependent manner.

### Example 2

### Preparation of Bifunctional Antibodies

Expression vectors of known cytosol-penetrating antibodies, known IL6R-binding antibodies, and known control antibodies without cytosol-penetrating ability shown in Table 2 were constructed by a method known in the art, and the nucleotide sequences of the obtained vectors were determined by a method known in the art. 3D8VH-G4T1E356K.Avi/hT4VL-KT0, 3D8VH-G4T1E356K.Avi/hT4VL03-KT0, 2C10VH-G4T1E356K.Avi/2C10VL-KT0, and MRAH-G4T1K439E.Avis/MRAL-KT0 are respectively abbreviated as 3D8, 3D8.03, 2C10, and MRA.

TABLE 2

| Name | Heavy chain | Light chain |
|---|---|---|
| Cytosol-penetrating antibody 3D8: | 3D8VH-G4T1E356K.Avi | Light chain hT4VL-KT0 |
| 3D8VH-G4T1E356K.Avi/hT4VL-KT0 | (SEQ ID NO: 31) | (SEQ ID NO: 32) |
| Cytosol-penetrating antibody 3D8.03: | 3D8VH-G4T1E356K.Avi | Light chain hT4VL03-KT0 |
| 3D8VH-G4T1E356K.Avi/hT4VL03-KT0 | (SEQ ID NO: 31) | (SEQ ID NO: 33) |
| Cytosol-penetrating antibody 2C10: | 2C10VH-G4T1E356K.Avi | Light chain 2C10VL-KT0 |
| 2C10VH-G4T1E356K.Avi/2C10VL-KT0 | (SEQ ID NO: 34) | (SEQ ID NO: 35) |
| IL6R-binding antibody MRA: | MRAH-G4T1K439E.Avi | MRAL-KT0 |
| MRAH-G4T1K439E.Avi/MRAL-KT0 | (SEQ ID NO: 36) | (SEQ ID NO: 37) |
| IL6R-binding antibody: | H237-G4TlK439E.Avi | L104-KT0 |
| I1237-G4T1K439E.Avi/L104-KT0 | (SEQ ID NO: 38) | (SEQ ID NO: 39) |
| Control antibody IC17: | IC17HdK-G4T1K439E.Avi | IC17L-KT0 |
| IC17HdK-G4T1K439E.Avi/IC17L-KT0 | (SEQ ID NO: 40) | (SEQ ID NO: 41) |
| Control antibody IC17: | IC17HdK-G4T1E356K.Avi | IC17L-KT0 |
| IC17HdK-G4T1E356K.Avi/IC17L-KT0 | (SEQ ID NO: 42) | (SEQ ID NO: 41) |

The expression vectors of the antibodies with the Avi tag fused to the C terminus of the heavy chain were constructed by linking the heavy chain-encoding gene with a gene encoding the Avi tag (Protein Science 1999, 8:921-929) which is a peptide sequence known to be biotinylated by biotin ligase (BirA).

The constructed expression vectors were transiently transfected with Expi293 cells (Thermo Fisher Scientific) to express the antibodies. The antibodies were purified from the obtained culture supernatants by a method known in the art using MabSelect SuRe pcc (5 mL) (GE Healthcare) and AKTA Xpress (GE Healthcare). The absorbance at 280 nm was measured using a spectrometer, and the concentration of the purified antibodies was calculated using the absorption coefficient calculated from the obtained value by the PACE method (Protein Science 1995; 4:2411-2423). The purified antibodies were concentrated using Amicon Ultra-4 (30K) (Merck Millipore) as necessary.

Bifunctional antibodies were prepared from the purified antibodies by a method known in the art. The names and the combinations of arms of the bifunctional antibodies are shown in Table 3. The bifunctional antibodies of 3D8VH-G4T1E356K.Avi/hT4VL03-KT0 with MRAH-G4T1K439E.Avi/MRAL-KT0, with H237-G4T1K439E.Avis/L104-KT0, and with IC17HdK-G4T1K439E.Avi/IC17L-KT0 are respectively abbreviated as 3D8.03//MRA, 3D8.03//SA, and 3D8.03//IC17. Meanwhile, the bifunctional antibodies prepared by combination with 2C10VH-G4T1E356K.Avi/2C10VL-KT0 instead of 3D8VH-G4T1E356K.Avi/hT4VL03-KT0 are respectively abbreviated as 2C10//MRA, 2C10//SA, and 2C10//IC17. Furthermore, the bifunctional antibodies prepared by combination of IC17HdK-G4T1E356K.Avi/IC17L-KT0 with MRAH-G4T1K439E.Avi/MRAL-KT0 and with H237-G4T1K439E.Avi/L104-KT0 are respectively abbreviated as IC17//MRA and IC17//SA.

TABLE 3

| Bifunctional | | |
|---|---|---|
| antibody | First arm | Second arm |
| 3D8.03//MRA | 3D8VH-G4T1E356K.Avi/hT4VL03-KT0 | MRAH-G4T1K439E.Avi/MRAL-KT0 |
| 3D8.03//SA | 3D8VH-G4T1E356K.Avi/hT4VL03-KT0 | H237-G4T1K439E.Avi/L104-KT0 |
| 3D8.03//IC17 | 3D8VH-G4T1E356K.Avi/hT4VL03-KT0 | IC17HdK-G4T1K439E.Avi/IC17L-KT0 |
| 2C10//MRA | 2C10VH-G4T1E356K.Avi/2C10VL-KT0 | MRAH-G4T1K439E.Avi/MRAL-KT0 |
| 2C10//SA | 2C10VH-G4T1E356K.Avi/2C10VL-KT0 | H237-G4T1K439E.Avi/L104-KT0 |
| 2C10//IC17 | 2C10VH-G4T1E356K.Avi/2C10VL-KT0 | IC17HdK-G4T1K439E.Avi/IC17L-KT0 |
| IC17//MRA | IC17HdK-G4T1E356K.Avi/IC17L-KT0 | MRAH-G4T1K439E.Avi/MRAL-KT0 |
| IC17//SA | IC17HdK-G4T1E356K.Avi/IC17L-KT0 | H237-G4T1K439E.Avi/L104-KT0 |

### Example 3

Construction of Target Cell Lines

A DNA in which the P2A peptide derived from porcine teschovirus-1 and GFP were added to the downstream of the human IL6R gene (Accession no. NP_000556) registered in GenPept was synthesized and cloned into the pCXND3 vector constructed by the inventor's company to prepare pCXND3-IL6R/P2A/GFP. Transfection was conducted using 2.5 micrograms (micro g) of the prepared vector with Lipofectamine 3000 (Invitrogen) for 3×105 cells of Flp-In-CHO cells (Invitrogen) which were cultured in Ham's F-12 medium (Gibco) containing 10% FBS (Sigma Aldrich) and 100 unit/mL penicillin—100 micro g/mL streptomycin (Gibco). From day 3 after the transfection, selection was carried out by adding Geneticin (Invtrogen) at a concentration of 400 to 500 micro g/mL to the medium. Selected cells were analyzed using FACSAria (BD Bioscience) to conduct single cloning of GFP-positive cells. The cell lines thus established were stained with the anti-human IL6R antibody PF-1 (prepared by the inventor's company) and a PE-labelled anti-human kappa chain antibody (SouthernBiotech) and analyzed again using FACSAria to confirm IL6R expression. In Example 4 described below, the Flp-In-CHO cell expressing IL6R is called IL6R+CHO cell, and the Flp-In-CHO cell is called CHO cell.

### Example 4

### Detection of Antibodies Translocated to the Cytosol by an Assay Using Biotin Ligase

To assess the cytosol translocation ability of each antibody prepared in Example 2, antibodies translocated to the cytosol were detected referring to the method described in W. P. R. Verdurmen et al., Journal of Controlled Release 200 (2015) 13-22. Specifically, in the method described below, cells expressing biotin ligase (BirA) and an Avi tag-fused antibody are incubated, and if the antibody is translocated to the cytosol, the Avi tag is biotinylated by BirA expressed in the cytosol. The quantity of the antibody present in the cytosol can be evaluated by detecting the biotinylated antibody with Streptavidin-HRP.

### (1) Cell-Antibody Reaction

Suspensions of CHO cells and IL6R+CHO cells prepared in Ham's F-12 Nutrient Mix (Gibco, Cat #11765-054) containing 10% FBS (Sigma Aldrich, Cat #182012-500ML) and Penicillin-Streptomycin (Gibco, Cat #15140-122) were seeded at 250 microliters (micro L)/well (IL6R+CHO: 3.0×10⁴ cells/well; CHO: 6×10⁴ cells/well) in Costar (registered trademark) 48 well cell culture cluster Flat bottom with Lid (Corning, Cat #3548), and this was cultured overnight under the conditions of 37 degrees Celsius (degrees C.) at 5% CO₂.

Next, the IL6R+CHO cells and CHO cells were transiently transfected with the expression vector of BirA (SEQ ID NO: 43) by a method known in the art using Opti-MEM I (lx) (Gibco, Cat #31985-062) and Lipofectamin 3000 kit (Life Technologies, Cat #L3000-015), and BirA was expressed by overnight culture under the conditions of 37 degrees C. at 5% CO₂.

After removing the culture supernatant, a medium containing the antibody (final concentration: 4 micromolar (micro M)), D-Biotin (Sigma Aldrich, Cat #B4501-10G) (final concentration: 0.1 mM), and MG-132 (Merck Millipore, Cat #474790-5MG) (final concentration: 50 micro M) was added at 225 micro L/well, and this was incubated at 37 degrees C. for 6 hours. The solution containing the antibody was removed using an aspirator, and cells were washed three times with 250 micro L of D-PBS(−). Accutase (Nacalai Tesque, Cat #12679-54) was added at 100 micro L/welland incubated at 37 degrees C. to detach cells from the culture flask. Then, a medium was added at 800 micro L/well, and the cells were collected in microtubes. The supernatant was removed by centrifugation (400×g, two minutes), and 800 micro L of D-PBS(−) was added. Centrifugation was conducted again, and the supernatant was removed. Sample Buffer Solution with 3-Mercapto-1,2-propanediol (Wako, Cat #199-16132) was mixed with an equal amount of MQ, and this was pre-heated at 96 degrees C. 10 micro L of this solution was added to the collected cells and heated at 96 degrees C. for eight minutes. Then, the samples were cooled on ice, and stored at −20 degrees C.

### (2) Detection of Biotinylated Antibodies

Biotinylated antibodies in the cell lysate prepared in (1) were detected using Simple Western Wes (Protein Simple) and 12-230 kDa Wes Separation Module, 8×25 capillary cartridges (Protein Simple, Cat #SM-W004). The prepared cell lysate was thawed at room temperature, and centrifuged (15,000 rpm, three minutes) to collect the supernatant. The supernatant was diluted 8-fold with MQ. Then, the diluted supernatant was mixed at a volume ratio of 4:1 with 5×Fluorescent Master Mix prepared by pre-mixing 20 micro L of 10×Sample Buffer, 20 micro L of 400 mM, and a tube of Fluorescent Standard, and heated at 95 degrees C. for five minutes. After mixing, the mixture was stored on ice and used as a measurement sample. HRP substrate was prepared by mixing 200 micro L of Luminol-S and 200 micro L of peroxide.

3 micro L of the measurement sample, 10 micro L of Antibody Diluent II, 10 micro L of Streptavidin-HRP, and 15 micro L of HRP substrate were respectively added to A, B, C, and E columns of Pre-filled Micro Plate of 12-230 kDa Wes Separation Module, 8×25 capillary cartridges, and measurement was conducted. The measurement was carried out together using as a molecular weight marker 5 micro L of Biotin Ladder prepared by pre-mixing 16 micro L of MilliQ, 2 2 micro L of 10×Sample Buffer, 2 micro L of 400 mM DTT, and a tube of Biotin Ladder. The reagents attached to Simple Western Wes (Protein Simple) and 12-230 kDa Wes Separation Module, 8×25 capillary cartridges (Protein Simple, Cat #SM-W004) and Biotin Detection Module for Wes, Peggy Sue or Sally Sue (Cat #DM-004) were used.

The measurement by Wes was conducted as follows: Separation Matrix Load Time, 200 seconds; Stacking Matrix Load Time, 15 seconds; Sample Load Time, 9 seconds; Separation Time, 25 seconds; Separation voltage, 375 V; Standards Exposure, 4 seconds; EE Immobilization Time, 230 seconds; Matrix washes, 3 times; Matrix Wash Soak Time, 150 seconds; Wash Soak Time, 150 seconds, Antibody Diluent Time, 5 minutes; Primary Antibody Time, 30 minutes; Washes, twice; Wash Soak Time, 150 seconds; and Detection Profile, HDR.

Actin contained in the measurement sample was detected using beta Actin (13E5) Rabbit mAb (HRP conjugate) (Cell Signaling, Cat #51255) diluted 50-fold with Antibody Diluent II, instead of Streptavidin-HRP. In this case, after thawing the cell lysate at room temperature, this was centrifuged (15,000 rpm, three minutes), and the collected supernatant was diluted 2-fold with MQ, and used to prepare a measurement sample.

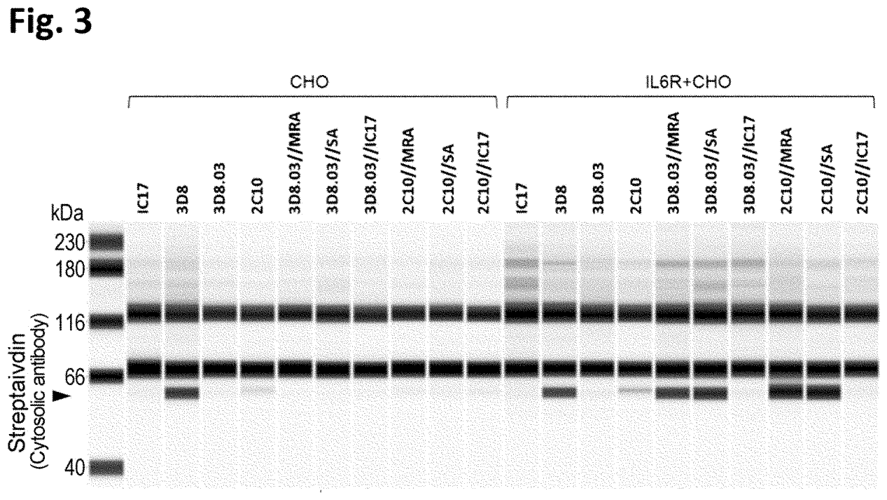

The detected HRP signals were analyzed using Compass for Simple Western Version 3.1.7 (Protein Simple), and the lane image as shown in FIG. 3 was generated.

As a result, 3D8 which is a known cytosol-penetrating antibody was detected in the lysates of CHO cells and IL6R+CHO cells, and it was confirmed that the antibody is non-specifically translocated to the cytosol regardless of the expression of the cell surface antigen (FIG. 3). On the other hand, 3D8.03 which is a 3D8 mutant lacking heparin sulfate proteoglycan (HSPG)-binding ability was not detected in the lysate of CHO cells or IL6R+CHO cells, and it was confirmed that the antibody cannot reach the cytosol of neither of the cell lines. Meanwhile, 3D8.03//MRA which is a bifunctional antibody from 3D8.03 and the anti-IL6R antibody was scarcely detected in the lysate of CHO cells, but provides a strong detection signal in the lysate of IL6R+CHO cells. 3D8.03//IC17 was not detected in the lysate of neither of the cell lines. These results indicate that, by providing the bifunctional antibody from 3D8.03 and the anti-IL6R antibody, the antibody can be delivered to the cytosol of target cells in an IL-6R-dependent manner.

Furthermore, similar results were obtained for bifunctional antibodies using 2C10. 2C10 was detected in the lysates of both of the cell lines, 2C10//MRA was detected with a strong detection signal only in the lysate of IL6R+CHO cells as opposed to CHO cells. Meanwhile, 2C10//IC17 was only scarcely detected in either of the cell lysates.

SA is a variant (pH-dependent antibody) in which a modification for allowing dissociation from the cell surface antigen IL6R under the acidic pH environment in endosome is introduced into MRA using a method known in the art (e.g., WO 2009/125825). For 3D8.03//SA and 2C10//SA, strong detection signals were also detected in the lysate of IL6R+CHO cells (FIG. 3). It is known that the half-life of an antibody in blood is prolonged by conferring pH dependency to an antibody binding to a cell surface antigen, and thus when pH dependency is provided for a cytosol-penetrating antigen-binding molecule, lower administration frequency and reduced dosage can be expected for the antigen-binding molecule. pH-dependent antibodies can be suitably prepared by those skilled in the art based on the disclosure of, for example, WO 2009/125825.

From the above results, it was demonstrated that, when a multifunctional antigen-binding molecule from a cytosol-penetrating antibody such as 3D8.03 and 2C10 and a cell surface antigen-binding antibody is provided, the antibody can be delivered to the cytosol of target cells in a cell surface antigen-dependent manner. In particular, 3D8.03 is an antibody that lacks binding ability to HSPG which is a cell surface antigen, and only has endosomal escape ability. Thus, when a multifunctional antigen-binding molecule in which such an endosomal escape antibody and a cell surface antigen-binding antibody are combined is provided, it is possible to efficiently deliver the multifunctional antigen-binding molecule to the cytosol of target cells, minimizing the delivery to non-target cells. It is thought that this technology can specifically deliver an antigen-binding molecule to the cytosol of target cells, and allow it to act on an antigen in the cytosol.

### Example 5

### Imaging Analysis of Antibodies in the Cytosol Using a Fluorescent Microscope

Imaging analysis was carried out using a fluorescent microscope for the antibodies prepared in Example 2.

Suspensions of Flp-In-CHO cells (Invitrogen) prepared in Ham's F-12 Nutrient Mix (Gibco, Cat #11765-054) containing 10% FBS (GE Healthcare Japan, Cat #SH30070.03) and Penicillin-Streptomycin (Gibco, Cat #15140-122), or Flp-In-CHO cells in which IL6R (GenPept Accession no. NP_000556) was overexpressed by a method known in the art, were seeded at 50 micro L/well (8.0×105 cells/mL and 4.0×105 cells/mL, respectively) in a 96-well plate (Corning, Cat #354649) whose bottom surface was coated with type I collagen. Cells were cultured overnight under the conditions of 37 degrees C. at 5% CO2. On the following day, the culture supernatant was removed, and a medium containing the antibody (final concentration: 3 micro M) and MG-132 (Merck Millipore, Cat #474790-5MG) (final concentration: 30 micro M) was added at 30 micro L/well, and this was incubated at 37 degrees C. for an hour. Next, the medium was removed from the sample, and this was washed with D-PBS(−). Furthermore, to remove antibodies remaining on the cellular membrane, washing was conducted for 30 seconds twice with a washing solution comprising 200 mM Glycine (Wako Pure Chemical Industries, Cat #077-00735)—HCl (Wako Pure Chemical Industries, Cat #083-01095), 150 mM NaCl (Wako Pure Chemical Industries, Cat #191-01665), pH 2.5. Then, cells were fixed by reacting 4% paraformaldehyde-phosphate buffer (Nacalai Tesque, Cat #09154-56) at room temperature for 15 minutes. Next, cells were permeabilized by overnight incubation at 4 degrees C. in PBS containing 0.5% Triton X-100 (Bio-Rad, Cat #161-0407) and 5% FBS. The solution used in the permeabilization was also used in the subsequent steps of incubation with the labelled antibody and washing. Cytosol translocated antibodies were labelled with 500-fold dilution of Goat Anti-Human IgG-TRITC (Thermo, Cat #A18810), by incubating at room temperature for an hour. In this step, to visualize the nucleus, Hoechst 33342 (Thermo, Cat #H3570) was diluted by 2000-fold and added thereto. Finally, cells were washed twice with PBS to prepare measurement samples. Sample measurement was conducted using IN Cell Analyzer 6000 (GE Healthcare).

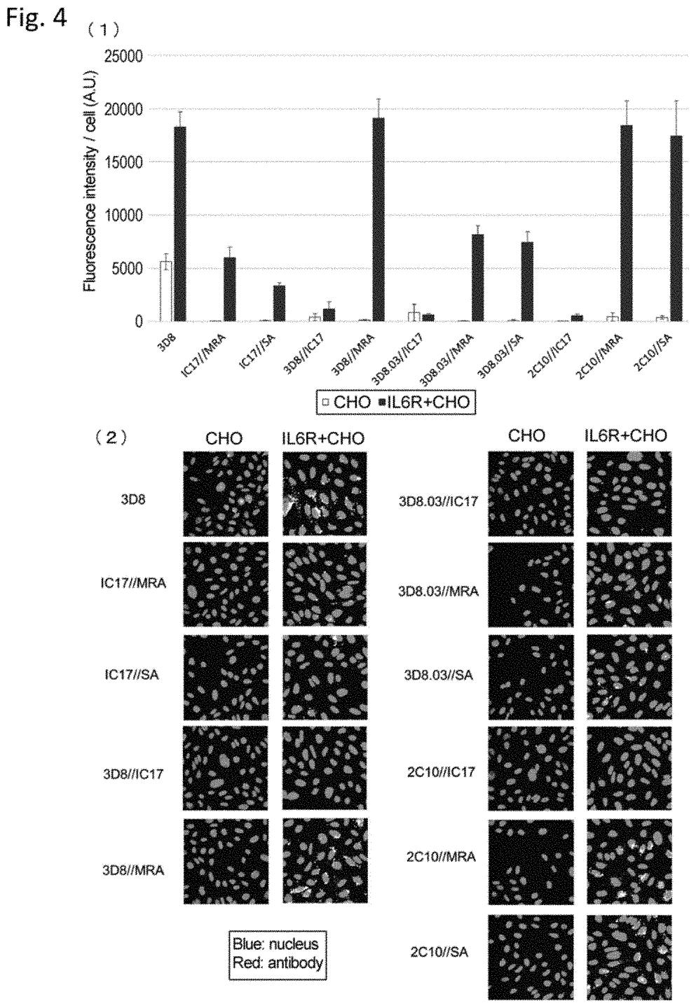

As a result, for 3D8//MRA, 3D8.03//MRA, 3D8.03//SA, 2C10//MRA, and 2C10//SA which are bifunctional antibodies of 3D8, 3D8.03, and 2C10, and the anti-IL6R antibody MRA or SA, only slight fluorescent signals were observed from CHO cells, whereas strong fluorescent signals were observed from IL6R+CHO cells. In IL6R+CHO cells, stronger fluorescent signals were detected for 3D8//MRA, 3D8.03//MRA, 3D8.03//SA, 2C10//MRA, and 2C10//SA, compared to 3D8HIC17, 3D8.03//IC17, 2C10//IC17, IC17//MRA, and IC17//SA. Furthermore, fluorescent signals were also observed from CHO cells for 3D8, and it was suggested that non-cell-specific cytosol penetration of the antibody occurred. For 3D8//MRA, cytosol penetration as with 3D8 occurred in IL6R+CHO cells, while almost no cytosol penetration was confirmed in CHO cells (FIG. 4). These results indicate that, when the bifunctional antibody from the cytosol-penetrating antibody such as 3D8 and the anti-IL6R antibody is provided, the antibody can be delivered to the cytosol of target cells in an IL6R-dependent manner, maintaining the cytosol-penetrating ability.

### Example 6

Assessment of the cytosol-penetrating ability of antibodies having an Fc region containing amino acid modifications that promote multimer formation

To produce hexamerized antibodies (hexabodies) for the known cytosol-penetrating antibodies and bifunctional antibodies described in Example 2, the antibody 3D8VH-G1 m.RGY/hT4VL03-KT0.Avi (3D8.03RGY.LAvi) (heavy chain: SEQ ID NO: 44; light chain: SEQ ID NO: 45) into which the R/G/Y modification (E345R/E430G/S440Y) (see WO2016/164480) was introduced into the heavy chain constant region by a method known in the art, was prepared. The Avi tag was fused with the C terminus of the light chain constant region, since if the Avi tag is fused to the C-terminus of the heavy chain constant region, antibody multimerization will be prevented. Furthermore, the antibody 3D8VH-G1m/hT4VL03-KT0.Avi (3D8.03.LAvi) (heavy chain: SEQ ID NO: 46; light chain: SEQ ID NO: 45) in which the Avi tag was fused with the C terminus of the light chain constant region, but no R/G/Y modification was introduced, was prepared. It has been reported that 3D8.03 shows cytosol-penetrating ability at pH 5.5 (Journal of Controlled Release 235 (2016) 165-175). Thus, 3D8.03RGY.LAvi and 3D8.03.LAvi were also incubated with cells under the condition of pH 5.5, and the cytosol-penetrating ability was assessed.

Suspensions of IL6R+CHO cells prepared in Ham's F-12 Nutrient Mix (Gibco, Cat #11765-054) containing 10% FBS (Sigma Aldrich, Cat #182012-500ML) and Penicillin-Streptomycin (Gibco, Cat #15140-122) were seeded at 250 micro L/well (IL6R+CHO: 3.0×10⁴ cells/well; CHO: 6×10⁴ cells/well) in Costar (registered trademark) 48 well cell culture cluster Flat bottom with Lid (Corning, Cat #3548), and this was cultured overnight under the conditions of 37 degrees C. at 5% CO₂.

Next, the IL6R+CHO cells were transiently transfected with the expression vector of BirA (SEQ ID NO: 43) by a method known in the art using Opti-MEM I (lx) (Gibco, Cat #31985-062) and Lipofectamine 3000 kit (Life Technologies, Cat #L3000-015), and BirA was expressed by overnight culture under the conditions of 37 degrees C. at 5% CO₂.

The antibody was added at a final concentration of 2, 4, or 10 micro M to a medium containing D-Biotin (Sigma Aldrich, Cat #B4501-10G) (final concentration: 0.1 mM), and MG-132 (Merck Millipore, Cat #474790-5MG) (final concentration: 50 micro M) supplemented with 0.5 M MES buffer (pH 4.5), to prepare a solution at pH 5.5. 225 micro L/well of the prepared solution was added to the cells from which the culture supernatant was removed, and this was incubated at 37 degrees C. for 6 hours. The solution containing the antibody was removed using an aspirator, and washing was carried out three times with 250 micro L of D-PBS(−). Accutase (Nacalai Tesque, Cat #12679-54) was added at 100 micro L/well, and this was incubated at 37 degrees C. Then, a medium was added at 800 micro L/well, and the cells were collected in microtubes. The supernatant was removed by centrifugation (400×g, two minutes), and 800 micro L of D-PBS(−) was added. Centrifugation was conducted again, and the supernatant was removed. Sample Buffer Solution with 3-Mercapto-1,2-propanediol (Wako, Cat #199-16132) was mixed with an equal amount of MQ, and this was pre-heated at 96 degrees C. 10 micro L of this solution was added to the collected cells, and this was heated at 96 degrees C. for eight minutes. Then, the samples were cooled on ice, and stored at −20 degrees C.

The detection of the biotinylated antibodies from the prepared cell lysates was performed according to the method described in Example 4-(2).

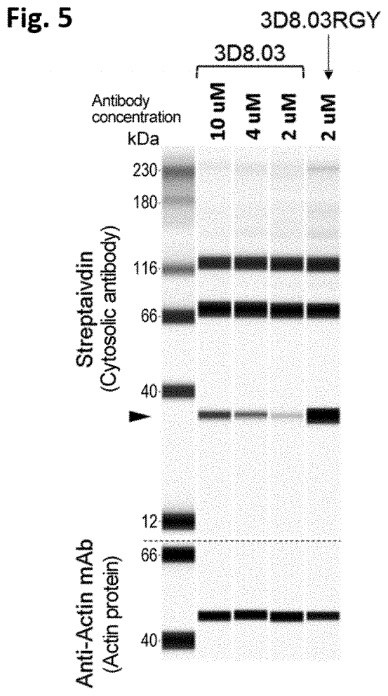

As a result, a strong detection signal was shown for a protein with a molecular weight corresponding to the light chain of the antibody fused with the Avi tag which was detected in the cell lysate incubated with 3D8.03RGY.LAvi, compared to the cell lysate incubated with 3D8.03.LAvi without the introduction of the RGY modification (FIG. 5). Furthermore, the detection signal was stronger for the protein corresponding to the antibody light chain when incubated with 2 micro M of 3D8.03RGY.LAvi, compared to when incubated with 10 micro M of 3D8.03.LAvi.

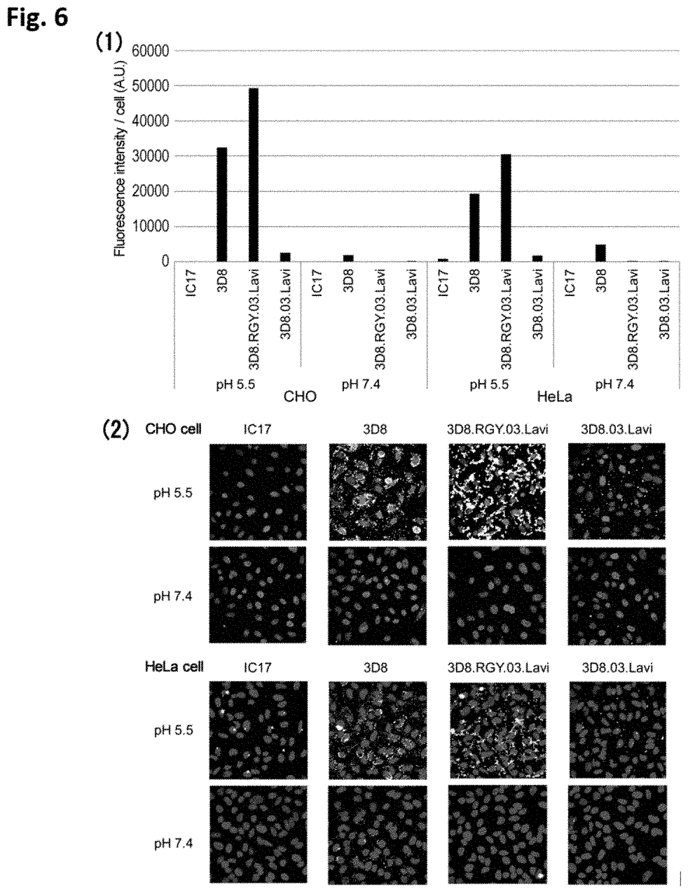

Furthermore, imaging analysis was performed using a microscope by the method described in Example 5. In addition to Flp-In-CHO cells (CHO cells) prepared by the method described in Example 5, HeLa cell suspensions were prepared in Minimum Essential Medium Eagle (Sigma, Cat #M4655-500ML) containing 10% FBS (Sigma Aldrich, Cat #182012-500ML) and Penicillin-Streptomycin (Gibco, Cat #15140-122). This was seeded at 50 micro L/well (Flp-In-CHO cells: 4.0×10⁴ cells/mL; HeLa: 3.0×10⁴ cells/mL) in a 96-well plate (Corning, Cat #354649) whose bottom surface was coated with type I collagen. This was cultured overnight under the conditions of 37 degrees C. at 5% CO₂, and used for the assay. The antibodies and the cells were incubated under the conditions where 0.5 M MES buffer (pH 4.5) was added to the cell suspensions to adjust pH to be 5.5, and the cytosol translocation of the antibodies was assessed. As a result, strong fluorescent signals were observed in cells incubated with 3D8.03RGY, compared to 3D8.03 without introduction of the RGY modification (FIG. 6). From these results, it was revealed that the cytosol-penetrating ability was enhanced in the cytosol-penetrating antibody introduced with the RGY modification which is known to promote hexamer formation. The above results show the possibility that the cytosol-penetrating ability can be enhanced by multimerization of a cytosol-penetrating antibody.

### Example 7

Assessment of the Target Cell-Specific Cytosol-Penetrating Ability of Antibodies Having an Fc Region Containing Amino Acid Modifications that Promote Multimer Formation

As mentioned above, HSPG-mediated endocytosis and interaction with a molecule on the membrane do not occur for 3D8.03 under neutral conditions, and thus the cytosol antigen-binding antibody, as it is, cannot be delivered to the cytosol. Meanwhile, the cytosol-penetrating ability is also expected to be enhanced if a 3D8. RGY antibody is similarly prepared using 3D8 which can be delivered to the cytosol under neutral conditions, and assessed; however, it is predicted that the target cell specificity will be attenuated. Thus, the heavy chain constant regions of bifunctional antibodies such as 3D8.03//MRA and 3D8//MRA are introduced with any of RGY(E345R/E430G/S440Y), RY(E345R/S440Y), RG(E345R/E430G), GY(E430G/S440Y), R(E345R) modification, or K428E/T437R modification (mAbs, 2017, 9, 7, 1129-1142), and antibodies that are present as a monomer in a solution, and form a hexamer only when they bind to an antigen, are prepared. Then, biotin ligase assay is carried out by the method described in Example 4-(2). As a result, in an IL6R+CHO cell lysate, a strong light emission signal of HRP is shown at the position of molecular weight corresponding to the antibody heavy or light chain linked to the Avi-tag, compared to the simple bifunctional antibodies, 3D8.03//MRA and 3D8//MRA. Similarly, as a result of performing imaging analysis by the method described in Example 5, a strong fluorescent signal is observed in IL6R+CHO cells compared to 3D8.03//MRA and 3D8//MRA. Accordingly, for these antibodies, it is shown that they form a hexamer and undergo endocytosis only when they bind to a target cell surface antigen, and they strongly interact with the membrane in the endosome, and thus the increase in target cell specificity and the enhancement of cytosol penetration can be achieved at the same time.

### Example 8

Assessment of the Cytosol-Penetrating Ability of Antibodies Produced by Making a Bifunctional Antibody with Enhanced Target Cell Specificity and Cytosol-Penetrating Ability to be Multivalent

By the assay utilizing biotin ligase and imaging analysis using a microscope, it is demonstrated that the target cell specificity and cytosol-penetrating ability are enhanced in an antibody in the molecular form in which a bifunctional antibody is made to be multivalent which is a molecular form described in FIG. 2, and other than the hexabody described in Examples 6 and 7.

The antibody is prepared by a method known in the art. Biotin ligase assay is carried out by the method described in Example 4-(2). As a result, in the molecular form shown in FIG. 2, a strong light emission signal of HRP is shown at the position of molecular weight corresponding to the antibody heavy or light chain linked to the Avi-tag, compared to the simple bifunctional antibody composed of a cytosol-penetrating antibody such as 3D8 or 3D8.03 and a cell surface antigen-binding antibody (FIG. 1a). Similarly, as a result of performing imaging analysis by the method described in Example 5, a strong fluorescent signal is observed compared to the simple bifunctional antibody. Accordingly, it is shown that the cell specificity and the cytosol-penetrating ability can be enhanced in various molecular forms described in FIG. 2 as with hexabody.

### Example 9

Detection of Bifunctional Antibodies with Anti-ASGPR Trafficked to the Cytosol by Split Nanoluc Assay

As another method to evaluate cytosol trafficking ability, it is known to utilize Nanoluc complementation (Schaub et al., Cancer Res. (2015) 75(23):5023, Dixon et al., ACS Chem Biol. (2016) 11(2):400). We created the improved method to reduce background signal, and tested bifunctional antibodies of known cytosol-penetrating antibodies and anti-ASGPR antibodies.

### (1) Concept of Split NanoLuc Assay

NanoLuc is a small 19 kDa luciferase enzyme engineered from a deep sea luminous shrimp. The split NanoLuc assay utilizes NanoLuc which is cleaved into two sub-units—a small 1.3 kDa Small BiT (SmBiT) and a larger 18 kDa Large BiT (LgBiT). Complementation of the LgBiT and SmBiT is observed as luminescence from, enzymatic reaction on substrate, in live cells by use of the NanoLuc Live Cell Assay System (Promega).

In the split NanoLuc assay, HeLa cells were genetically modified to stably express NanoLuc LgBiT intracellularly. The NanoLuc LgBiT was either expressed as free cytosolic protein, or tethered to an intracellular structure within the cytosol, for instance, the mitochondrial outer membrane. The tethering of the NanoLuc LgBiT to the mitochondrial outer membrane resulted in significantly lower background signal and better assay sensitivity compared to when NanoLuc LgBiT was expressed as free cytosolic protein. This was despite the tethered NanoLuc LgBiT having an overall lower expression as determined by GFP. The SmBiT conjugate was expressed on the antibodies tested. Penetration of antibody-SmBiT conjugates into the cytosol would complement the NanoLuc LgBiT luciferase enzyme, which could be detected by enhancement of luminescence.

### (2) Cell Line Construction

To generate HeLa cells stably expressing human ASGPR, cells were transfected with plasmids encoding human ASGPR-H1 (SEQ ID NO: 47) and ASGPR-H2 (SEQ ID NO: 48) subunits, in a ratio of 2:1 respectively, using Lipofectamine 3000 (Invitrogen). ASGPR-H1 and ASGPR-H2 were cloned into the plasmid pCXND3 which uses the CAG promoter and encodes neomycin antibiotic resistant gene. Antibiotic resistant cells were enriched further for high expression of ASGPR by cell sorting on FACSAria III (BD). The enriched cells were then isolated by limiting dilution and plating, and identified as single cell clones by microscopy (Solentim, Cell Metric CLD).

To generate cells expressing both ASGPR and eGFP-Nanoluc LgBiT, and cells expressing ASGPR and AKAP1-eGFP-NanoLuc LgBiT, HeLa cells stably expressing ASGPR were nucleofected using Cell Line Optimization 4D-Nucleofector™ X Kit (Lonza) and 4D-Nucleofector Core unit and 4D-Nucleofector X unit (Lonza), using the preset nucleofection program for HeLa. The LgBiT constructs were cloned into the plasmid pCXZD1 which uses the CAG promoter and encodes zeocin antibiotic resistant gene. The first was NanoLuc LgBit conjugated to the C-terminal of eGFP (eGFP-NanoLuc LgBit; SEQ ID NO: 49). The second was with an additional mitochondrial kinase anchoring protein 1 (AKAP1) conjugated to the N-terminal of eGFP (AKAP1-eGFP-NanoLuc LgBit; SEQ ID NO: 50). The antibiotic resistant cells were enriched further for high expression of eGFP by cell sorting on FACSAria III (BD). The enriched cells were then isolated by limiting dilution and plating, and identified as single cell clones by microscopy (Solentim, Cell Metric CLD).

HeLa cells were cultured in a standard culture media of Minimum Essential Media (Gibco) with 10% v/v Fetal Bovine Serum, 1% v/v MEM Non-Essential Amino Acids Solution (Gibco), 1% v/v Sodium Pyruvate (Gibco) and 1% v/v Penicillin-Streptomycin (Gibco). HeLa clones expressing Asialoglycoprotein receptor (ASGPR) were cultured with standard culture media supplemented with 1 mg/mL Geneticin (G418 Sulfate) (Gibco). HeLa cells expressing ASGPR and eGFP-NanoLuc LgBiT, or ASGPR and AKAP1-eGFP-NanoLuc LgBiT, were cultured in standard culture media with 1 mg/mL Geneticin and 600 micro g/mL Zeocin (Invivogen). All cell types were cultured in a humidified 5% CO₂, 37 degrees C. incubator.

### (3) Assay Set Conditions

HeLa cells expressing ASGPR and eGFP-NanoLuc LgBiT (hereafter termed as HeLa-Nluc), and HeLa cells expressing ASGPR and AKAP1-eGFP-Nanoluc LgBiT (hereafter termed as HeLa-AKAP1_Nluc) were rinsed with DPBS, no calcium, no magnesium (Gibco). The cells were then trypsinized with 0.25% Trypsin-EDTA (Gibco) at room temperature until detached from culture flask. Trypsin was then neutralized in excess culture media containing 10% FBS. Cells were pelleted at 200×g for 5 minutes at 4 degrees C., and rinsed twice with chilled Opti-MEM (Gibco). The cells were suspended in chilled Opti-MEM at a concentration of 1×10⁶ cells/mL, and kept cold for up to 30 minutes before use.

Antibodies to be tested were diluted in Histidine Buffer, 20 mM His-HCl+150 mM NaCl (Nacalai Tesque), to 11-times of the test concentrations. 5-times substrate from the NanoLuc Live Cell Assay System (Promega, Cat #N2011) was prepared at room temperature.

1×10⁵ cells was transferred at 100 micro L per well (Perkin Elmer, Cat #6005688), and 10 micro L of 11-times antibodies were added directly into cells and mixed by pipette. 5-times substrate was then added to the cells and antibody mixture immediately after mixing.

The plate was immediately placed in a pre-warmed 37 degrees C. luminescence plate reader (Promega, Glomax Explorer). A 2-step read cycle was done every 5 minutes for 12 cycles—the plate was shaken for 1 minute at 400 rpm, before reading luminescence.

### (4) Antibody Preparation

Expression vectors of known cytosol-penetrating antibodies and anti-ASGPR-binding antibodies shown in Table 4 were constructed by a method known in the art, and the nucleotide sequences of the obtained vectors were determined by a method known in the art. The expression vectors of the antibodies with the SmBiT fused to the C terminus of the heavy chain were constructed by linking the heavy chain-encoding gene with a gene encoding the SmBiT.AGA0078Ha-SG1.S3p.SmBiT/AGA0078L0030-kOMC is a variant of AGA0078Ha-SG1.S3p.SmBiT/AGA0078La-kOMC, which allowed to dissociate from ASGPR in the acidic environment such as endosome using a method known in the art (e.g., WO 2009/125825). 3D8H-SG1.S3n.SmBiT/hT4VL-SK1, 2C10VH-SG1. S3n. SmBiT/2C10VL-SK1, AGA0078Ha-SG1. S3p. SmBiT/AGA0078La-kOMC, AGA0078Ha-SG1.S3p.SmBiT/AGA0078L0030-kOMC and IC17HdK-SG1.S3p.SmBiT/IC17L-SK1 are abbreviated as 3D8, 2C10, NpH_ASGPR, pH_ASGPR and IC17dK respectively.

TABLE 4

| Antibody name | Heavy chain | Light chain |
|---|---|---|
| Cytosol penetrating antibody, 3D8: | 3D8H-SG1.S3n.SmBiT | hT4VL-SK1 |
| 3D8H-SG1.S3n.SmBiT/hT4VL-SK1 | (SEQ ID NO: 51) | (SEQ ID NO: 52) |
| Cytosol penetrating antibody, 2C10: | 2C10VH-SG1.S3n.SmBiT | 2C10VL-SK1 |
| 2C10VH-SG1.S3n.SmBiT/2C10VL-SK1 | (SEQ ID NO: 53) | (SEQ ID NO: 54) |
| Anti-ASGPR antibody, NpH_ASGPR: | AGA0078Ha-SG1.S3p.SmBiT | AGA0078La-k0MC |
| AGA0078Ha-SG1.S3p.SmBiT/AGA0078La-k0MC | (SEQ ID NO: 55) | (SEQ ID NO: 56) |
| Anti-ASGPR antibody, pH_ASGPR: | AGA0078Ha-SG1.S3p.SmBiT | AGA0078L0030-k0MC |
| AGA0078Ha-SG1.S3p.SmBiT/AGA0078L0030-k0MC | (SEQ ID NO: 55) | (SEQ ID NO: 57) |
| Negative control antibody IC17dK: | IC17HdK-SG1.S3n.SmBiT | IC17L-SK1 |
| IC17HdK-SG1.S3n.SmBiT/IC17L-SK1 | (SEQ ID NO: 58) | (SEQ ID NO: 59) |
| Negative control antibody IC17dK: | IC17HdK-SG1.S3p.SmBiT | IC17L-SK1 |
| IC17HdK-SG1.S3p.SmBiT/IC17L-SK1 | (SEQ ID NO: 60) | (SEQ ID NO: 59) |

For antibody preparation, heavy chain and light chain plasmids were transiently transfected in expi293-F cells (Thermo scientific) according to the manufacturer's instructions. Recombinant antibodies were purified with protein A (GE Healthcare) affinity chromatography and eluted in D-PBS, Tris Buffered Saline (TBS), or His buffer (20 mM Histidine, 150 mM NaCl, pH6.0). Eluates were further subjected to Size exclusion chromatography using Superdex 200 column (GE healthcare) to remove high molecular weight and/or low molecular weight components. The purified antibodies were concentrated using Amicon Ultra-4 (30K) (Merck Millipore) as necessary.

Bifunctional antibodies were prepared from the purified antibodies by a method known in the art. The names and the combinations of arms of the bifunctional antibodies are shown in Table 5. The bifunctional antibodies of 3D8H-SG1.S3n.SmBiT/hT4VL-SK1 with IC17HdK-SG1.S3p.SmBiT/IC17L-SK1, with AGA0078Ha-SG1.S3p.SmBiT/AGA0078La-kOMC, and with AGA0078Ha-SG1.S3p.SmBiT/AGA0078L0030-kOMC are respectively abbreviated as IC17dK//3D8, NpH_ASGPR//3D8 and pH_ASGRP//3D8. Meanwhile, the bifunctional antibodies prepared by combination with 2C10VH-SG1.S3n.SmBiT/2C10VL-SK1 instead of 3D8H-SG1.S3n.SmBiT/hT4VL-SK1 are respectively abbreviated as IC17dK//2C10, NpH_ASGPR//2C10 and pH_ASGPR//2C10. Furthermore, the bifunctional antibodies prepared by combination of IC17HdK-SG1.S3n.SmBiT/IC17L-SK1 with AGA0078Ha-SG1.S3p.SmBiT/AGA0078La-kOMC, with AGA0078Ha-SG1.S3p.SmBiT/AGA0078L0030-kOMC and with IC17HdK-SG1.S3p.SmBiT/IC17L-SK1 are respectively abbreviated as NpH_ASGPR//IC17dK, pH_ASGPR//IC17dK and IC17dK//IC17dK.

TABLE 5

| Bi-functional antibody | First atm | Second atm |
|---|---|---|
| IC17dK//3D8 | IC17HdK-SG1.S3p.SmBiT/IC17L-SK1 | 3D8H-SG1.S3n.SmBiT/hT4VL-SK1 |
| NpH_ASGPR//3D8 | AGA0078Ha-SG1.S3p.SmBiT/AGA0078La-k0MC | 3D8H-SG1.S3n.SmBiT/hT4VL-SK1 |
| pH_ASGPR//3D8 | AGA0078Ha-SG1.S3p.SmBiT/AGA0078L0030-k0MC | 3D8H-SG1.S3n.SmBiT/hT4VL-SK1 |
| IC17dK//2C10 | IC17HdK-SG1.S3p.SmBiT/IC17L-SK1 | 2C10VH-SG1.S3n.SmBiT/2C10VL-SK1 |
| NpH_ASGPR//2C10 | AGA0078Ha-SG1.S3p.SmBiT/AGA0078La-k0MC | 2C10VH-SG1.S3n.SmBiT/2C10VL-SK1 |
| pH_ASGPR//2C10 | AGA0078Ha-SG1.S3p.SmBiT/AGA0078L0030-k0MC | 2C10VH-SG1.S3n.SmBiT/2C10VL-SK1 |
| IC17dK//IC17dK | IC17HdK-SG1.S3p.SmBiT/IC17L-SK1 | IC17HdK-SG1.S3n.SmBiT/IC17L-SK1 |
| NpH_ASGPR//IC17dK | AGA0078Ha-SG1.S3p.SmBiT/AGA0078La-k0MC | IC17HdK-SG1.S3n.SmBiT/IC17L-SK1 |
| pH_ASGPR//IC17dK | AGA0078Ha-SG1.S3p.SmBiT/AGA0078L0030-k0MC | IC17HdK-SG1.S3n.SmBiT/IC17L-SK1 |

### (5) Results

To compare the sensitivity of the split Nanoluc cell lines generated, cytosol targeting antibodies 3D8 and 2C10 were tested in eGFP-NanoLuc LgBiT and AKAP1-eGFP-Nanoluc LgBiT HeLa cell lines. To evaluate background signal in each cell line, soluble SmBiT peptide (SEQ ID NO: 63) was added. The molar concentration of 3D8, 2C10 and SmBiT peptide used were identical at 0.2 micromolar. To enable comparison, the luciferase signal for each cell line was normalized to histidine buffer condition and set as 1.0 fold change of luciferase signal was calculated by taking reference to histidine buffer.

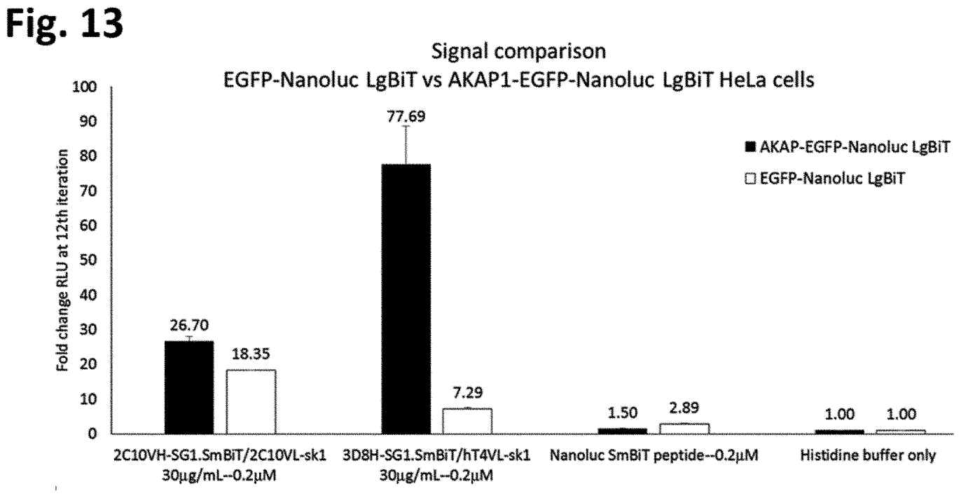

By comparing the signal of Nanoluc SmBiT peptide, the AKAP1-eGFP-Nanoluc LgBiT HeLa cell line showed lower background compared eGFP-NanoLuc LgBiT (FIG. 13). In addition, the fold change of cytosol penetrating antibodies 3D8 and 2C10 were also higher in the AKAP1-eGFP-Nanoluc LgBiT HeLa cell line.

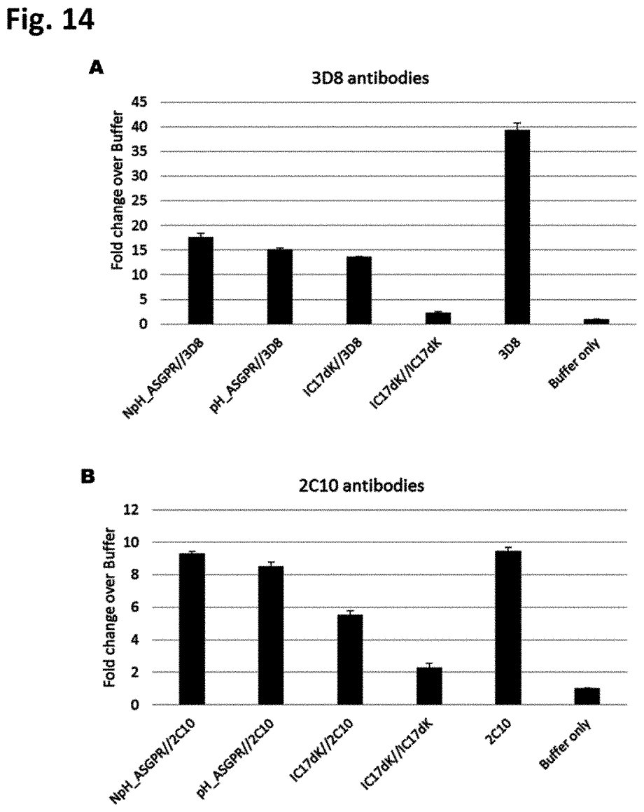

As for the evaluation of bifunctional antibodies, the fold change of monovalent form of cytosol penetrating antibodies such as IC17dK//3D8 and IC17dK//2C10 were lower than bivalent form of 3D8 and 2C10 suggesting that monovalent form was less penetrated into the cytosol. However, bifunctional antibodies that target ASGPR and have cell penetrating arms such as NpH_ASGPR//3D8, pH_ASGPR//3D8, NpH_ASGPR//2C10 and pH_ASGPR//2C10 showed higher fold change than that of monovalent form of cytosol-penetrating antibodies IC17dK//3D8 and IC17//2C10 (FIG. 14A and FIG. 14B). From those results, bifunctional antibodies internalize into cells by ASGPR mediated manner and escape from endosome by cytosol penetrating antibody function.

### Example 10

### Detection of Bifunctional Antibodies Towards IL6R and ASGPR Expressing Cell Lines by Biotin Ligase Assay

### (10-1) Cell Line Construction

### (1) HeLa/BirA

To generate HeLa cells stably expressing BirA, Hela cells (ATCC, Cat #CCL-2) were transfected with plasmids encoding BirA (SEQ ID NO: 43) using Lipofectamine 3000 (Theromo Fisher Scientific). BirA was cloned into the plasmid pCXND3 which uses the CAG promoter and encodes neomycin antibiotic resistant gene. Antibiotic resistant cells were enriched further for high expression of BirA by cell sorting on FACSAria IIu sorter (Becton Dickinson). The enriched cells were then isolated by limiting dilution and plating, and identified as single cell clones by microscopy.

Selected clones were then expanded. BirA expression was evaluated by capillary immunoassay using Simple Western Wes (Protein Simple) with 12-230 kDa Wes Separation Module, 8×25 capillary cartridges (Protein Simple, Cat #SM-W004), 1/500 diluted anti-BirA antibody (Novus Biologicals, Cat #NBP2-59938) and Anti-Mouse Detection Module for Wes, Peggy Sue or Sally Sue (Protein Simple, Cat #DM-002). The BirA expression was confirmed using Compass for Simple Western Version 3.1.7 (Protein Simple), and a cell clone showing high BirA expression was selected.

### (2) HeLa/BirA/IL6R

To generate HeLa cells stably expressing BirA (SEQ ID NO: 43) and human IL6R (Accession no. NP_000556), the IL6R gene was cloned into the plasmid pCXZD1 which uses the CAG promoter and encodes zeocin antibiotic resistant gene. HeLa/BirA cells were transfected with pCXZD1_IL6R plasmid using Lipofectamine 3000 (Thermo Fisher Scientific).

To select cells stably expressing IL6R, antibiotic resistant cells were incubated with 1 micro M of anti-IL6R antibody PF1H-G4T1K439E.Avi/PF1L-KT0 (heavy chain, PF1H-G4T1K439E.Avi (SEQ ID NO: 66); light chain, PF1L-KT0 (SEQ ID NO: 67) in MACS buffer (Auto MACS Rinsing Solution (Miltenyi Biotec, Cat #130-091-222) and MACS BSA Stock Solution (Miltenyi Biotec, Cat #1130-091-376)) for 30 min on ice. After wash in MACS buffer, cells were further incubated with R-phycoerythrin (PE) labelled anti-kappa antibody (Southern Biotech, Cat #2060-09) for 30 min on ice in MACS buffer. After wash in MACS buffer, cells expressing IL6R were sorted by FACSAria IIu sorter (Becton Dickinson). Selected clones were then expanded. Flow cytometry using anti-IL6R antibody PF1H-G4T1K439E.Avi/PF1L-KT0 and R-phycoerythrin (PE) labelled anti-kappa antibody (Southern Biotech, Cat #2060-09) with FACS Verse (Becton Dickinson) confirmed high expression of IL6R. BirA expression was also confirmed by capillary immunoassay using Simple Western Wes (Protein Simple).

### (3) HeLa/ASGPR

To generate HeLa cells stably expressing human ASGPR, cells were transfected with plasmids encoding human ASGPR-H1 SEQ ID NO: 47 and ASGPR-H2 SEQ ID: 48 subunits, in a ratio of 2:1 respectively, using Lipofectamine 3000 (Thermo Fisher Scientific). ASGPR-H1 and ASGPR-H2 were cloned into the plasmid pCXND3 which uses the CAG promoter and encodes neomycin antibiotic resistant gene. Antibiotic resistant cells were enriched further for high expression of ASGPR by cell sorting on FACSAria III (Becton Dickinson). The enriched cells were then isolated by limiting dilution and plating, and identified as single cell clones by microscopy (Solentim, Cell Metric CLD).

### (4) HeLa/ASGPR/BirA

To generate HeLa cells stably expressing BirA (SEQ ID NO: 43) and human ASGPR (ASGPR-H1 (SEQ ID NO: 47) and ASGPR-H2 (SEQ ID NO: 48)), the BirA gene was cloned into the plasmid pCXZD1 which uses the CAG promoter and encodes zeocin antibiotic resistant gene. HeLa/ASGPR cells were transfected with pCXZD1_BirA plasmid using Lipofectamine 3000 (Thermo Fisher Scientific). Antibiotic resistant cells were enriched further for high expression of BirA by cell sorting on FACSAria Ilu sorter (Becton Dickinson). The enriched cells were then isolated by limiting dilution and plating, and identified as single cell clones by microscopy.

Selected clones were then expanded. Flow cytometry analysis using FACS Verse (Becton Dickinson) with anti-ASGPR antibody AGA0008Ha-G4T1K439E.Avi/AGA0008La-kOMC (heavy chain, AGA0008Ha-G4T1K439E.Avi (SEQ ID NO: 68); light chain, AGA0008La-kOMC (SEQ ID NO: 69)) and Goat Anti-Human IgG-Alexa Fluor 647 (Southern Biotech, Cat #2040-31) confirmed ASGPR expression. BirA expression was also confirmed by capillary immunoassay using Simple Western Wes (Protein Simple).

For assays, HeLa/BirA cells were cultured in Minimum Essential Medium Eagle (Sigma Aldrich, Cat #M4655-500ML) containing 10% FBS (Sigma Aldrich, Cat #172012-500ML), Penicillin-Streptomycin (Gibco, Cat #15140-122) and 750 micro g/mL Geneticin (Cat #10131-027, Thermo Fisher Scientific). HeLa/BirA/IL6R and HeLa/ASGPR/BirA cells were cultured in Minimum Essential Medium Eagle (Sigma, Cat #M4655-500ML) containing 10% FBS (Sigma Aldrich, Cat #172012-500ML), Penicillin-Streptomycin (Gibco, Cat #15140-122), 750 micro g/mL Geneticin (Thermo Fisher Scientific, Cat #10131-027) and 100 micro g/mL of zeocin (Thermo Fisher Scientific, Cat #R25001).

### (10-2)Biotin Ligase (BirA) Assay

Biotin ligase (BirA) assay assessed the cytosol trafficking of bifunctional antibodies that target a receptor expressed on cell surface. Bifunctional antibodies with a cell penetrating arm and a receptor binding arm were incubated target cells which express BirA in cytosol and receptor antigen on cell surface. When bifunctional antibodies fused with Avitag on C-terminus of light chains penetrated into cytosol, antibodies were biotinylated. Then those biotinylated antibodies in cytosol were detected from cell lysate with streptavidin-HRP by capillary immunoassay using Simple Western Wes (Protein Simple).

### (10-3) Cell-Antibody Reaction

Antibodies listed in Table 6 were used to prepare bifunctional antibodies listed in Table 7 as described in Example 2. Avitag was fused to C-terminus of light chains of those antibodies. Suspensions of HeLa/BirA/IL6R and HeLa/ASGPR/BirA were seeded at 100 micro L/well (0.2×10⁵ cells/well) in Costar 96 well cell culture plate (Corning, Cat #3596). Then cells were cultured in a humidified 5% CO₂, 37 degrees C. incubator overnight. Then culture supernatant was removed by aspirator and cells were washed with 150 micro L/well of D-PBS(−). 3 micro M of antibody samples prepared in the culture medium containing 0.1 mM D-Biotin (Sigma Aldrich, Cat #B4501-10G) and 30 micro M MG-132 (Merck Millipore, Cat #474790-5MG) were added to the cells (50 micro L/well) and incubated for 3 hrs in a humidified 5% CO₂, 37 degrees C. incubator. Then, antibody solutions were removed by aspirator and cells were washed twice in 150 micro L of ice-cold D-PBS(−). Then 30 micro L of RIPA buffer (Thermo Fisher Scientific, Cat #89900) containing 1×Protease & Phosphatase Inhibitor Cocktail (Thermo Fisher Scientific, Cat #1861281), 5 mM EDTA (Thermo Fisher Scientific, Cat #1861274) and 100 micro M of Avitag peptide (SEQ ID NO: 90) (Genscript), which was acetylated on N-terminal and amidated on C-terminal, was added to cells and incubated on ice for 5 min to prepare cell lysate. Then, cell lysate was collected and stored at −80 degree C. until detection step.

Biotinylated antibodies in the cell lysate were detected by capillary immune assay using Simple Western Wes (Protein Simple) and 12-230 kDa Wes Separation Module, 8×25 capillary cartridges (Protein Simple, Cat #SM-W004). The cell lysate was mixed at a volume ratio of 4:1 with 5-times Fluorescent Master Mix prepared by pre-mixing 20 micro L of 10-times Sample Buffer, 20 micro L of 400 mM DTT, and a tube of Fluorescent Standard. Then samples were heated at 95 degrees C. for 5 min. Then the mixture was stored on ice as a measurement sample. HRP substrate was prepared by mixing 200 micro L of Luminol-S and 200 micro L of peroxide. 3 micro L of the measurement sample, 10 micro L of Antibody Diluent II, 10 micro L of Streptavidin-HRP, and 15 micro L of HRP substrate were applied to A, B, C and E columns of Pre-filled Micro Plate of 12-230 kDa Wes Separation Module, 8×25 capillary cartridges, and measurement was conducted. As a molecular weight marker, 5 micro L of Biotin Ladder prepared by pre-mixing 16 micro L of MQ, 2 micro L of 10×Sample Buffer, 2 micro L of 400 mM DTT and a tube of Biotin Ladder was used. Those reagents were used from 12-230 kDa Wes Separation Module, 8×25 capillary cartridges (Protein Simple, Cat #SM-W004) and Biotin Detection Module for Wes, Peggy Sue or Sally Sue (Protein Simple, Cat #DM-004). To detect total amount of antibody in cell lysate, Anti-Human IgG Detection Module for Wes, Peggy Sue or Sally Sue (Protein Simple, Cat #DM-005) and Anti-Human IgG Secondary HRP Antibody (Protein Simple, Cat #043-491) were used. To detect HSP90 as internal standard in cell lysate, 1/50 diluted Rabbit anti-HSP90 mAb (C45G5) HRP conjugate (Cell Signaling Technologies, Cat #79641) prepared in Antibody Diluent II was used.

The measurement by Simple Western Wes (Protein Simple) was conducted as follows: Separation Matrix Load Time, 200 seconds; Stacking Matrix Load Time, 15 seconds; Sample Load Time, 9 seconds; Separation Time, 25 seconds; Separation voltage, 375 V; Standards Exposure, 4 seconds; EE Immobilization Time, 230 seconds; Matrix washes, 3 times; Matrix Wash Soak Time, 150 seconds; Wash Soak Time, 150 seconds, Antibody Diluent Time, 5 minutes; Primary Antibody Time, 30 minutes; Washes, twice; Wash Soak Time, 150 seconds; and Detection Profile, HDR.

The detected HRP signals were analyzed using Compass for Simple Western Version 3.1.7 (Protein Simple).

TABLE 6

| Antibody name | Heavy chain | Ligh chain |
|---|---|---|
| Cytosol penetrating Ab, 3D8: | 3D3VH-G4T1E356K | hT4VL-KT0.Avi |
| 3D8VH-G4T1E356K/hT4VL-KT0.Avi | (SEQ ID NO: 70) | (SEQ ID NO: 71) |
| Cytosol penetrating Ab, 3D8.03: | 3D8VH-G4T1E356K | hT4VL03-KT0.Avi |
| 3D8VH-G4T1E356K/hT4VL03-KT0.Avi | (SEQ ID NO: 70) | (SEQ ID NO: 72) |
| Cytosol penetrating Ab, 2C10: | 2C10VH-G4T1E356K | 2C10VL-KT0.Avi |
| 2C10VH-G4T1E356K/2C10VL-KT0.Avi | (SEQ ID NO: 73) | (SEQ ID NO: 74) |
| Cytosol penetrating Ab, m3E10VH.D31N: | m3E10VH.D31N-G4T1E356K | m3E10VL-KT0.Avi |
| m3E10VH.D31N-G4T1E356K/m3E10VL-KT0.Avi | (SEQ ID NO: 75) | (SEQ ID NO: 76) |
| Anti-IL6R antibody, MRA: | MRAH-G4T1K439E | MRAL-KT0.Avi |
| MRAH-G4T1K439E/MRAL-KT0.Avi | (SEQ ID NO: 77) | (SEQ ID NO: 78) |
| Anti-IL6R antibody, H237: | H237-G4T1K439E | L104-KT0.Avi |
| H237-G4T1K439E/L104-KT0.Avi | (SEQ ID NO: 79) | (SEQ ID NO: 80) |
| Anti-ASGPR antibody, AGA0008: | AGA0008Ha-G4T1K439E | AGA0008La-KT0.Avi |
| AGA0008Ha-G4T1K439E/AGA0008La-KT0.Avi | (SEQ ID NO: 81) | (SEQ ID NO: 82) |
| Anti-ASGPR antibody, A0841: | A0841H0217-G4T1K439E | A0811L0125-KT0.Avi |
| A0841H0217-G4T1K439E/A0811L0125-KT0.Avi | (SEQ ID NO: 83) | (SEQ ID NO: 84) |
| Negative control Ab IC17dKp: | IC17HdK-G4T1E356K | IC17L-KT0.Avi |
| IC17HdK-G4T1E356K/IC17L-KT0.Avi | (SEQ ID NO: 85) | (SEQ ID NO: 86) |
| Negative control Ab IC17dKn: | IC17HdK-G4T1K439E | IC17L-KT0.Avi |
| IC17HdK-G4T1K439E/IC17L-KT0.Avi | (SEQ ID NO: 87) | (SEQ ID NO: 86) |

TABLE 7

| Bifunctional antibody | First arm | Second arm |
|---|---|---|
| IC17dKp//MRA | IC17HdK-G4T1E356K/IC17L-KT0.Avi | MRAH-G4T1K439E/MRAL-KT0.Avi |
| IC17dKp//H237 | IC17HdK-G4T1E356K/IC17L-KT0.Avi | H237-G4T1K439E/L104-KT0.Avi |
| IC17dKp//AGA0008 | IC17HdK-G4T1E356K/IC17L-KT0.Avi | AGA0008Ha-G4T1K439E/AGA0008La-KT0.Avi |
| IC17dKp//A0841 | IC17HdK-G4T1E356K/IC17L-KT0.Avi | A0841H0217-G4T1K439E/A0811L0125-KT0.Avi |
| 3D8//IC17dKn | 3D8VH-G4T1E356K/hT4VL-KT0.Avi | IC17HdK-G4T1K439E/IC17L-KT0.Avi |
| 3D8//MRA | 3D8VH-G4T1E356K/hT4VL-KT0.Avi | MRAH-G4T1K439E/MRAL-KT0.Avi |
| 3D8//H237 | 3D8VH-G4T1E356K/hT4VL-KT0.Avi | H237-G4T1K439E/L104-KT0.Avi |
| 3D8//AGA0008 | 3D8VH-G4T1E356K/hT4VL-KT0.Avi | AGA0008Ha-G4T1K439E/AGA0008La-KT0.Avi |
| 3D8//A0841 | 3D8VH-G4T1E356K/hT4VL-KT0.Avi | A0841H0217-G4T1K439E/A0811L0125-KT0.Avi |
| 3D8.03//IC17dKn | 3D8VH-G4T1E356K/hT4VL03-KT0.Avi | IC17HdK-G4T1K439E/IC17L-KT0.Avi |
| 3D8.03//MRA | 3D8VH-G4T1E356K/hT4VL03-KT0.Avi | MRAH-G4T1K439E/MRAL-KT0.Avi |
| 3D8.03//H237 | 3D8VH-G4T1E356K/hT4VL03-KT0.Avi | H237-G4T1K439E/L104-KT0.Avi |
| 3D8.03//AGA0008 | 3D8VH-G4T1E356K/hT4VL03-KT0.Avi | AGA0008Ha-G4T1K439E/AGA0008La-KT0.Avi |
| 3D8.03//A0841 | 3D8VH-G4T1E356K/hT4VL03-KT0.Avi | A0841H0217-G4T1K439E/A0811L0125-KT0.Avi |
| 2C10//IC17dKn | 2C10VH-G4T1E356K/2C10VL-KT0.Avi | IC17HdK-G4T1K439E/IC17L-KT0.Avi |
| 2C10//MRA | 2C10VH-G4T1E356K/2C10VL-KT0.Avi | MRAH-G4T1K439E/MRAL-KT0.Avi |
| 2C10//H237 | 2C10VH-G4T1E356K/2C10VL-KT0.Avi | H237-G4T1K439E/L104-KT0.Avi |
| 2C10//AGA0008 | 2C10VH-G4T1E356K/2C10VL-KT0.Avi | AGA0008Ha-G4T1K439E/AGA0008La-KT0.Avi |
| 2C10//A0841 | 2C10VH-G4T1E356K/2C10VL-KT0.Avi | A0841H0217-G4T1K439E/A0811L0125-KT0.Avi |
| m3E10.D31N//IC17dKn | m3E10VH.D31N-G4T1E356K/m3E10VL-KT0.Avi | IC17HdK-G4T1K439E/IC17L-KT0.Avi |
| m3E10.D31N//MRA | m3E10VH.D31N-G4T1E356K/m3E10VL-KT0.Avi | MRAH-G4T1K439E/MRAL-KT0.Avi |
| m3E10.D31N//H237 | m3E10VH.D31N-G4T1E356K/m3E10VL-KT0.Avi | H237-G4T1K439E/L104-KT0.Avi |
| m3E10.D31N//AGA0008 | m3E10VH.D31N-G4T1E356K/m3E10VL-KT0.Avi | AGA0008Ha-G4T1K439E/AGA0008La-KT0.Avi |
| m3E10.D31N//A0841 | m3E10VH.D31N-G4T1E356K/m3E10VL-KT0.Avi | A0841H0217-G4T1K439E/A0811L0125-KT0.Avi |

### (10-4) Result

BirA assay assessed cytosol penetration of bifunctional antibodies.

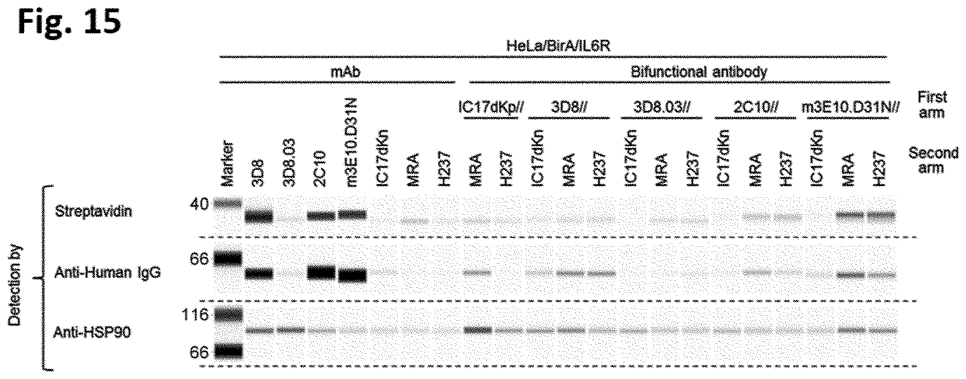

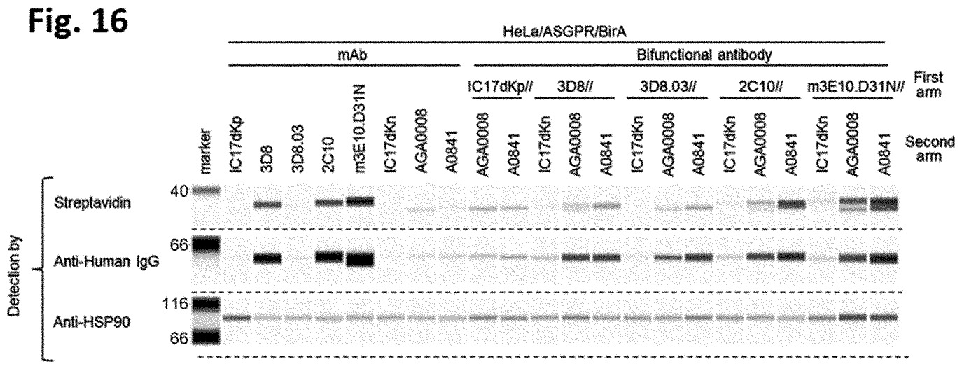

Biotinylated light chains of bivalent (mAb) format of cell penetrating antibodies such as 3D8, 2C10 and m3E10.D31N were detected by streptavidin-HRP from cell lysates of both HeLa/BirA/IL6R and HeLa/ASGPR/BirA (FIG. 15 and FIG. 16). However, monovalent format of cytosol penetrating antibodies such as 3D8//IC17dKn, 2C10//IC17dKn and m3E10.D31NHIC17dKn in which first arm is cytosol penetrating antibody and second arm is KLH binding antibody IC17dKn showed lower signals than bivalent format when detected by both streptavidin-HRP and anti-human IgG (FIG. 15 and FIG. 16). This result suggests that monovalent format of cell penetrating antibodies less internalized into cytosol than bivalent format of those antibodies.

To evaluate receptor dependent cytosol trafficking, bifunctional antibodies were prepared using anti-IL6R antibody (MRA) and pH dependent anti-IL6R antibody (H237). Bifunctional antibodies composed of cell penetrating antibodies and IL6R binding antibodies (3D8//MRA, 3D8//H237, 3D8.03//MRA, 3D8.03//H237, 2C10//MRA, 2C10//H237, m3E10.D31N//MRA and m3E10.D31N//H237) showed higher signal in HeLa/BirA/IL6R when detected by streptavidin HRP and anti-human IgG than 3D8//IC17dKn, 3D8.03//IC17dKn, 2C10//IC17dKn and m3E10.D31NHIC17dKn (FIG. 15). Those results indicate that bifunctional antibodies with a cell penetrating antibody arm and IL6R binding arm showed cytosol trafficking in IL6R dependent manner.

In HeLa/ASGPR/BirA cells, bifunctional antibodies showed ASGPR dependent cytosol trafficking as well. To target ASGPR, bifunctional antibodies were prepared using anti-ASGPR antibody (AGA0008) and pH dependent anti-ASGPR antibody (A0841). Bifunctional antibodies composed of cell penetrating antibodies and ASGPR binding antibodies (3D8//AGA0008, 3D8//A0841, 3D8.03//AGA0008, 3D8.03//A0841, 2C10//AGA0008, 2C10//A0841, m3E10.D31NHAGA0008 and m3E10.D31N//A0841) showed higher signal when detected by streptavidin HRP and anti-human IgG than 3D8//IC17dKn, 3D8.03//IC17dKn, 2C10//IC17dKn and m3E10.D31NHIC17dKn (FIG. 16). Those results indicate that bifunctional antibodies internalize into cells by ASGPR dependent manner and showed cytosol trafficking ability.

### Example 11

### Imaging Analysis of Cytosol Trafficking of Bifunctional Antibodies in IL6R and ASGPR Expressing Cell Lines

Imaging analysis was conducted to evaluate cytosol penetrating ability of bifunctional antibodies in receptor dependent manner. Antibodies listed in Table 8 were used to prepare bifunctional antibodies listed in Table 9 as described in Example 2. To target IL6R, anti-IL6R antibodies MRA and H54/L28 and pH dependent anti-IL6R antibody H237 were used. To target ASGPR, anti-ASGPR antibody AGA0008 was used.

Suspensions of HeLa/BirA, HeLa/BirA/IL6R and HeLa/ASGPR/BirA were seeded at 50 micro L/well (7.5×10⁵ cells/well) in BioCoat Collagen I Multiwell Plates 96-well Black/clear (Corning, Cat #354649). Then, cells were cultured for 24 hours in a humidified 5% CO₂, 37 degrees C. incubator. After removing the culture supernatant, cells were incubated with 3 micro M of antibodies in 30 micro L/well of culture medium for 3 hours in a humidified 5% CO₂, 37 degrees C. incubator. After incubation, cells were washed twice in 200 mM Glycine (Wako Pure Chemical Industries, Cat #077-00735)—HCl(Wako Pure Chemical Industries, Cat #083-01095), 150 mM NaCl (Wako Pure Chemical Industries, Cat #191-01665), pH 2.5. Then, cells were fixed by 4% paraformaldehyde-phosphate buffer (Nacalai Tesque, Cat #09154-56) at room temperature for 15 minutes and permeabilized by PBS containing 0.5% Triton X-100 (Bio-Rad, Cat #161-0407) and 5% FBS. The solution used in the permeabilization was also used in the subsequent steps for incubating with the secondary antibody and washing. Cells were incubated with 1/500-diluted Goat Anti-Human IgG-TRITC (Thermo Fisher Scientific, Cat #A18810) and 1/2000-diluted Hoechst 33342 (Thermo Fisher Scientific, Cat #H3570) for 1 hour at room temperature. After washing twice, cells were subjected to imaging analysis using IN Cell Analyzer 6000 (GE Healthcare) with UV (excitation wave length at 375 nm) and Green laser (excitation wave length at 561 nm).

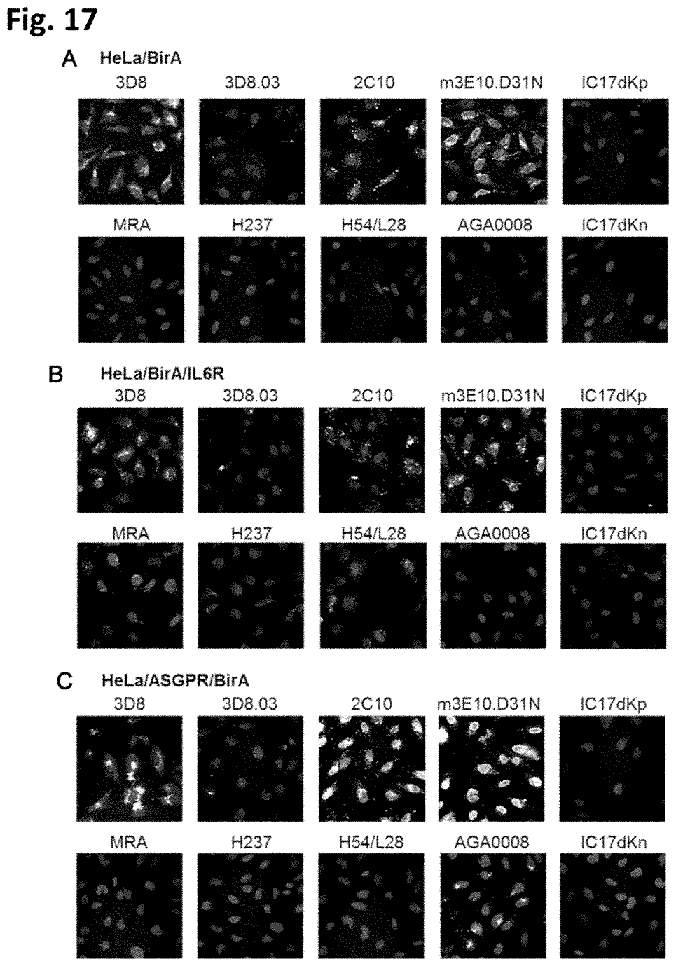

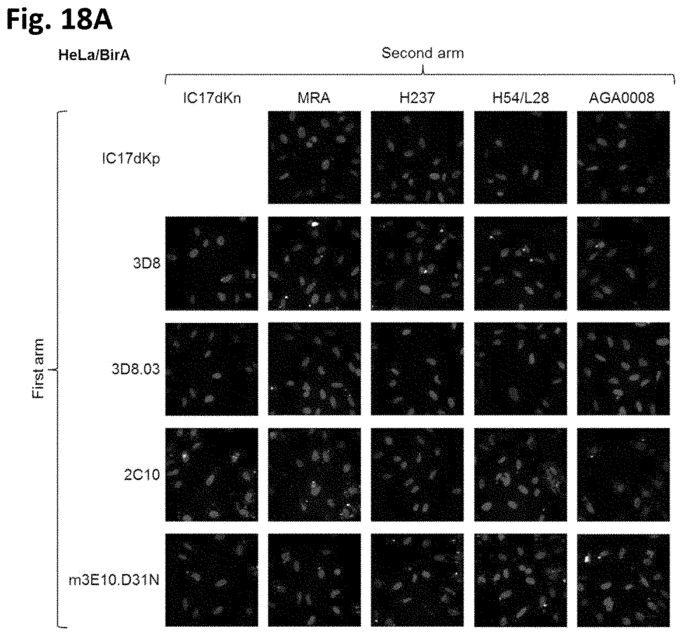

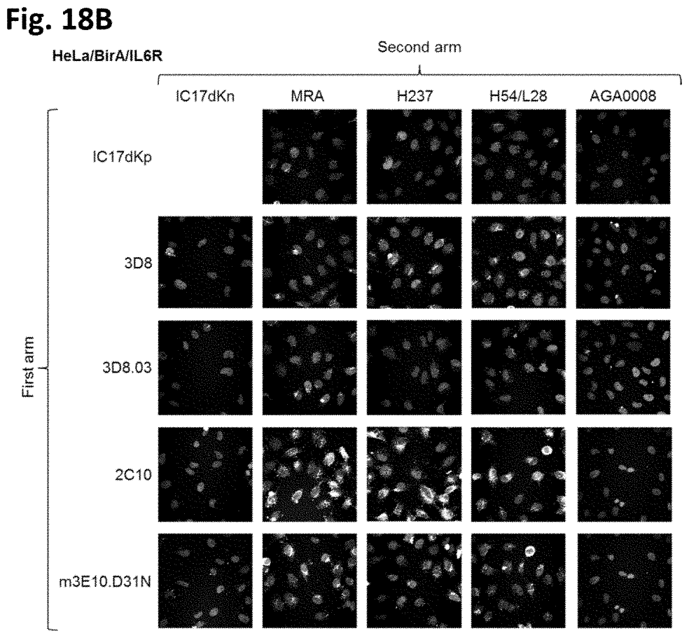

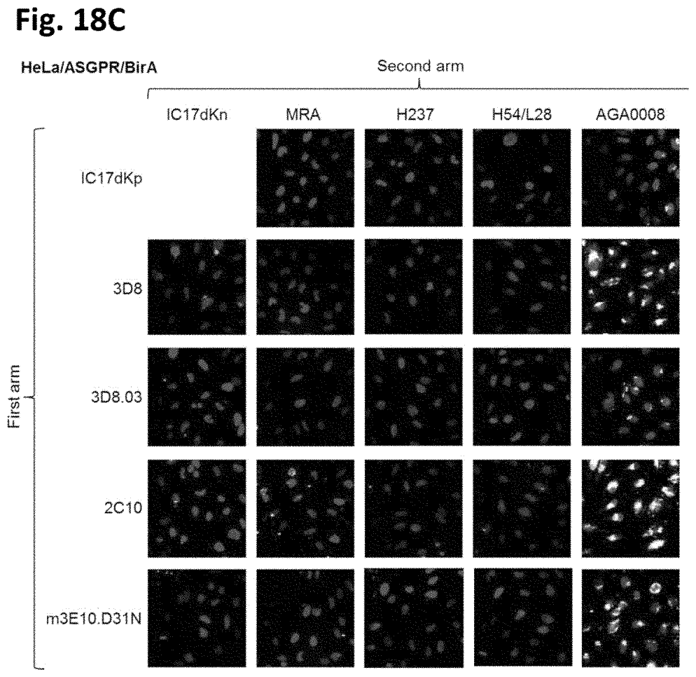

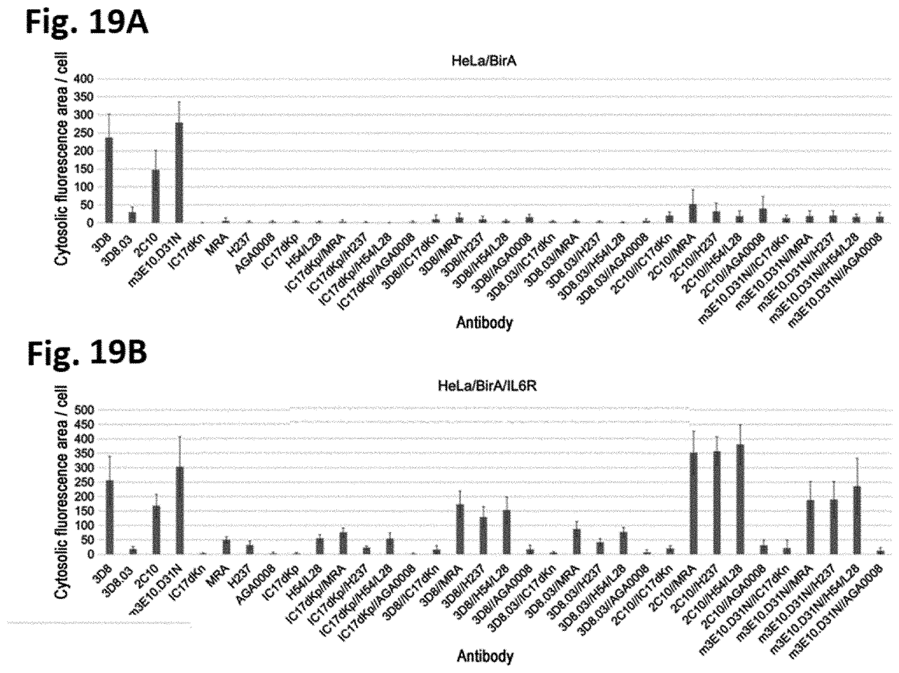

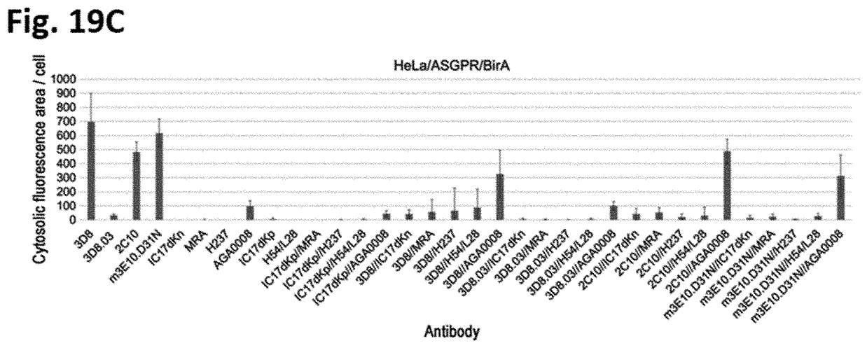

Fluorescence signals of TRITC and Hoechst33342 were merged and shown in FIGS. 17 and 18. Cytosolic fluorescence were assessed as average area size of fluorescence signal per cell (FIG. 19).

As shown in FIG. 17 and FIG. 19, cytosol penetration of antibodies such as 3D8, 2C10 and m3E10.D31N were detected from cytosol of HeLa/BirA, HeLa/BirA/IL6R and HeLa/ASGPR/BirA. Notably, 2C10 and m3E10.D31N were localized not only in cytosol but also with nucleus.

Antibodies against IL6R (MRA, H237 and H54/L27) and ASGPR (AGA0008) were detected from Hela/BirA/IL6R and HeLa/ASGPR/BirA, respectively. However those receptor binding antibodies showed vesicle like fluorescence suggesting those antibodies were entrapped in cellular compartments such as endosomes and were not escaping from endosomes.

Monovalent form of cytosol penetrating antibodies composed of one arm of cell penetrating antibody and one arm of KLH binding antibody IC17dKn (3D8//IC17dKn, 2C10//IC17dKn and m3E10.D31N//IC17dKn) showed less signals than bivalent format of 3D8, 2C10 and m3E10.D31N indicating that two arms of cytosol penetrating antibodies were required for internalization (FIGS. 18A to 18C and FIG. 19A to 19C).

Bifunctional antibodies that target IL6R (3D8//MRA, 3D8//H237, 3D8//H54/L28, 2C10//MRA, 2C10//H237, 2C10//H54/L28, m3E10.D31N//MRA, m3E10.D31N//H237 and m3E10.D31N//H54/L28) showed cytosol penetration in HeLa/BirA/IL6R (FIG. 18B and FIG. 19B). Those results indicate that bifunctional antibodies internalize into cells by IL6R mediated manner and escape from endosome by cell penetrating antibody function.

Bifunctional antibodies targeting ASGPR showed receptor mediated internalization and cell penetration activity as well. Bifunctional antibodies that target ASGPR (3D8//AGA0008, 2C10//AGA0008 and m3E10.D31N//AGA0008) showed fluorescence signal in cytosol (FIG. 18C and FIG. 19C). Those results indicate that bifunctional antibodies internalize into cells by ASGPR mediated manner and escape from endosome by cell penetrating antibody function.

Bifunctional antibodies that target IL6R (3D8//MRA, 3D8//H237, 3D8//H54/L28, 2C10//MRA, 2C10//H237, 2C10//H54/L28, m3E10.D31N//MRA, m3E10.D31N//H237 and m3E10.D31N//H54/L28) and ASGPR (3D8//AGA0008, 2C10//AGA0008 and m3E10.D31N//AGA0008) did not show efficient internalization into HeLa/BirA not expressing target receptors (FIG. 18A and FIG. 19A). This result indicates that the bifunctional antibody format can reduce off target cell penetration of antibody to cells not expressing target receptors.

Some of those bifunctional antibodies such as 2C10//MRA, 2C10//H237, 2C10//H54/L28, m3E10.D31N//MRA, m3E10.D31N//H237, m3E10.D31N//H54/L28, 2C10//AGA0008 and m3E10.D31N//AGA0008 were localized not only to cytosol but also to nucleus, while the bivalent form of cell penetrating antibodies 2C10 and m3E10.D31N showed localization to nucleus. Those results suggest that bifunctional antibodies composed of a cell penetrating antibody and a receptor binding antibody can deliver antibody to nucleus as well as cytosol by receptor mediated manner.

The bifunctional antibody formats achieved receptor specific delivery of antibodies into cytosol. From those results, the bifunctional antibody formats achieved receptor specific delivery of antibodies to cytosol and nucleus.

TABLE 8

| Antibody name | Heavy chain | Ligh chain |
|---|---|---|
| Cytosol penetrating Ab, 3D8: | 3D8VH-G4T1E356K | hT4VL-KT0.Avi |
| 3D8VH-G4T1E356K/hT4VL-KT0.Avi | (SEQ ID NO: 70) | (SEQ ID NO: 71) |
| Cytosol penetrating Ab, 3D8.03: | 3D8VH-G4T1E356K | hT4VL03-KT0.Avi |
| 3D8VH-G4T1E356K/hT4VL03-KT0.Avi | (SEQ ID NO: 70) | (SEQ ID NO: 72) |
| Cytosol penetrating Ab, 2C10: | 2C10VH-G4T1E356K | 2C10VL-KT0.Avi |
| 2C10VH-G4T1E356K/2C10VL-KT0.Avi | (SEQ ID NO: 73) | (SEQ ID NO: 74) |
| Cytosol penetrating Ab, m3E10VH.D31N: | m3E10VH.D31N-G4T1E356K | m3E10VL-KT0.Avi |
| m3E10VH.D31N-G4T1E356K/m3E10VL-KT0.Avi | (SEQ ID NO: 75) | (SEQ ID NO: 76) |
| Anti-IL6R antibody, MRA: | MRAH-G4T1K439E | MRAL-KT0.Avi |
| MRAH-G4T1K439E/MRAL-KT0.Avi | (SEQ ID NO: 77) | (SEQ ID NO: 78) |
| Anti-IL6R antibody, H237: | H237-G4T1K439E | L104-KT0.Avi |
| H237-G4T1K439E/L104-KT0.Avi | (SEQ ID NO: 79) | (SEQ ID NO: 80) |
| Anti-IL6R antibody, H54/L28: | H54-G4T1K439E | L28-KT0.Avi |
| H54-G4T1K439E/L28-KT0.Avi | (SEQ ID NO: 88) | (SEQ ID NO: 89) |
| Anti-ASGPR antibody, AGA0008: | AGA0008Ha-G4T1K439E | AGA0008La-KT0.Avi |
| AGA0008Ha-G4T1K439E/AGA0008La-KT0.Avi | (SEQ ID NO: 81) | (SEQ ID NO: 82) |
| Negative control Ab IC17dKp: | IC17HdK-G4T1E356K | IC17L-KT0.Avi |
| IC17HdK-G4T1E356K/IC17L-KT0.Avi | (SEQ ID NO: 85) | (SEQ ID NO: 86) |
| Negative control Ab IC17dKn: | IC17HdK-G4T1K439E | IC17L-KT0.Avis |
| IC17HdK-G4T1K439E/IC17L-KT0.Avi | (SEQ ID NO: 87) | (SEQ ID NO: 86) |

TABLE 9

| Bifunctional antibody | First arm | Second arm |
|---|---|---|
| IC17dKp//MRA | IC17HdK-G4T1E356K/IC17L-KT0.Avi | MRAH-G4T1K439E/MRAL-KT0.Avi |
| IC17dKp//H237 | IC17HdK-G4T1E356K/IC17L-KT0.Avi | H237-G4T1K439E/L104-KT0.Avi |
| IC17dKp//AGA0008 | IC17HdK-G4T1E356K/IC17L-KT0.Avi | AGA0008Ha-G4T1K439E/AGA0008La-KT0.Avi |
| 3D8//IC17dKn | 3D8VH-G4T1E356K/hT4VL-KT0.Avi | IC17HdK-G4T1K439E/IC17L-KT0.Avi |
| 3D8//MRA | 3D8VH-G4T1E356K/hT4VL-KT0.Avi | MRAH-G4T1K439E/MRAL-KT0.Avi |
| 3D8//H237 | 3D8VH-G4T1E356K/hT4VL-KT0.Avi | H237-G4T1K439E/L104-KT0.Avi |
| 3D8//H54/L28 | 3D8VH-G4T1E356K/hT4VL-KT0.Avi | H54-G4T1K439E/L28-KT0.Avi |
| 3D8//AGA0008 | 3D8VH-G4T1E356K/hT4VL-KT0.Avi | AGA0008Ha-G4T1K439E/AGA0008La-KT0.Avi |
| 3D8.03//IC17dKn | 3D8VH-G4T1E356K/hT4VL03-KT0.Avi | IC17HdK-G4T1K439E/IC17L-KT0.Avi |
| 3D8.03//MRA | 3D8VH-G4T1E356K/hT4VL03-KT0.Avi | MRAH-G4T1K439E/MRAL-KT0.Avi |
| 3D8.03//H237 | 3D8VH-G4T1E356K/hT4VL03-KT0.Avi | H237-G4T1K439E/L104-KT0.Avi |
| 3D8.03//H54/L28 | 3D8VH-G4T1E356K/hT4VL03-KT0.Avi | H54-G4T1K439E/L28-KT0.Avi |
| 3D8.03//AGA0008 | 3D8VH-G4T1E356K/hT4VL03-KT0.Avi | AGA0008Ha-G4T1K439E/AGA0008La-KT0.Avi |
| 2C10//IC17dKn | 2C10VH-G4T1E356K/2C10VL-KT0.Avi | IC17HdK-G4T1K439E/IC17L-KT0.Avi |
| 2C10//MRA | 2C10VH-G4T1E356K/2C10VL-KT0.Avi | MRAH-G4T1K439E/MRAL-KT0.Avi |
| 2C10//H237 | 2C10VH-G4T1E356K/2C10VL-KT0.Avi | H237-G4T1K439E/L104-KT0.Avi |
| 2C10//H54/L28 | 2C10VH-G4T1E356K/2C10VL-KT0.Avi | H54-G4T1K439E/L28-KT0.Avi |
| 2C10//AGA0008 | 2C10VH-G4T1E356K/2C10VL-KT0.Avi | AGA0008Ha-G4T1K439E/AGA0008La-KT0.Avi |
| m3E10.D31N//IC17dKn | m3E10VH.D31N-G4T1E356K/m3E10VL-KT0.Avi | IC17HdK-G4T1K439E/IC17L-KT0.Avi |
| m3E10.D31N//MRA | m3E10VH.D31N-G4T1E356K/m3E10VL-KT0.Avi | MRAH-G4T1K439E/MRAL-KT0.Avi |
| m3E10.D31N//H237 | m3E10VH.D31N-G4T1E356K/m3E10VL-KT0.Avi | H237-G4T1K439E/L104-KT0.Avi |
| m3E10.D31N//H54/L28 | m3E10VH.D31N-G4T1E356K/m3E10VL-KT0.Avi | H54-G4T1K439E/L28-KT0.Avi |
| m3E10.D31N//AGA0008 | m3E10VH.D31N-G4T1E356K/m3E10VL-KT0.Avi | AGA0008Ha-G4T1K439E/AGA0008La-KT0.Avi |

### Example 12

Imaging Analysis of Cytosol Trafficking of Bifunctional Antibodies Conjugated with a Cargo in IL6R and ASGPR Expressing Cell Lines.

To assess delivery of a cargo conjugated to the bifunctional antibodies, streptavidin-AlexaFluor647 was conjugated to biotinylated antibodies and subjected to imaging analysis. Biotinylated antibodies were prepared by transient expression of antibody and BirA in Expi293 cells in a biotin supplemented culture medium. Biotinylated antibodies listed in Table 10 were purified as described in Example 2 and used to prepare biotinylated bifunctional antibodies listed in Table 11. Biotinylated bifunctional antibodies were mixed with equimolar amount of Streptavidin-AlexaFluor647 (Thermo Fisher Scientific, Cat #S32357) and incubated with HeLa/BirA, HeLa/BirA/IL6R and HeLa/ASGPR/BirA cells at final antibody concentration of 3 micro M. Imaging analysis was conducted as described in Example 11. IN Cell Analyzer 6000 (GE Healthcare) with UV (excitation wave length at 375 nm), blue laser (excitation wave length at 488 nm) and red laser (excitation wave length at 641 nm) was used to detect Hoechst33342, TRITC labeled anti-human IgG and AlexaFluor647 labeled streptavidin, respectively.

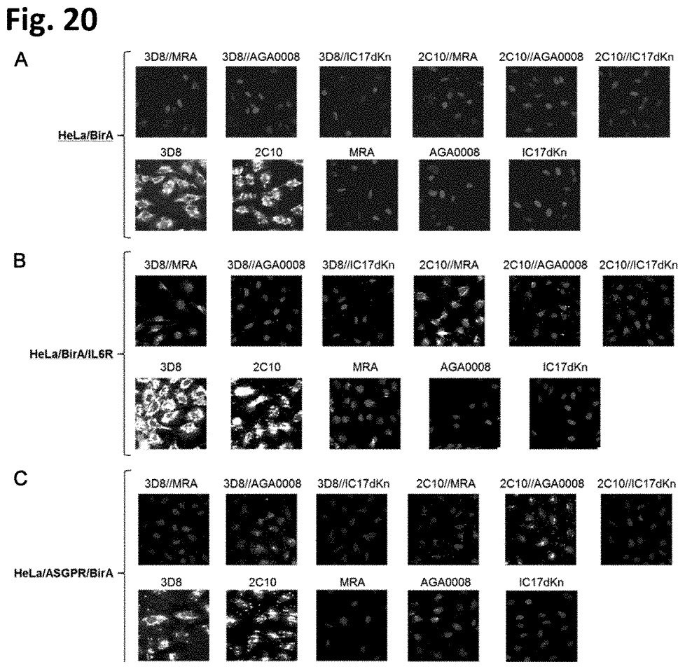

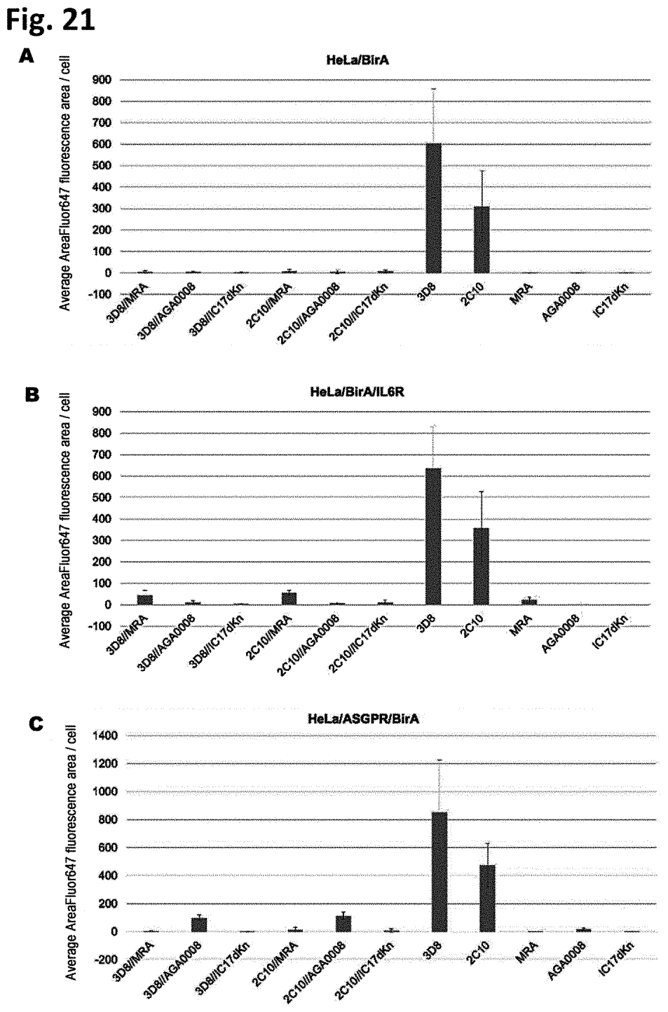

As shown in FIG. 20 and FIG. 21, bifunctional antibodies targeting IL6R (3D8//MRA and 2C10//MRA) and ASGPR (3D8//AGA0008 and 2C10//AGA0008) showed higher fluorescence signals of AlexaFluor647 in HeLa/BirA/IL6R and HeLa/ASGPR/BirA, respectively, than control antibodies 3D8//IC17dKn and 2C10//IC17dKn. Those bifunctional antibodies did not show fluorescence signals in HeLa/BirA cells which did not express target receptors. This result indicates that strepatavidin-AlexaFluor647 was delivered to target cells by conjugating to the bifunctional antibodies composed of a cytosol penetrating arm and a receptor binding arm. Those results suggest that the bifunctional antibodies can deliver cargo molecules to cytosol and nucleus of target cells which express target receptor antigens.

TABLE 10

| Antibody name | Heavy chain | Ligh chain |
|---|---|---|
| Cytosol penetrating Ab, 3D8: | 3D8VH-G4T1E356K | hT4VL-KT0.Avi |
| 3D8VH-G4T1E356K/hT4VL-KT0.Avi | (SEQ ID NO: 70) | (SEQ ID NO: 71) |
| Cytosol penetrating Ab, 2C10: | 2C10VH-G4T1E356K | 2C10VL-KT0.Avi |
| 2C10VH-G4T1E356K/2C10VL-KT0.Avi | (SEQ ID NO: 73) | (SEQ ID NO: 74) |
| Anti-IL6R antibody, MRA: | MRAH-G4T1K439E | MRAL-KT0.Avi |
| MRAH-G4T1K439E/MRAL-KT0.Avi | (SEQ ID NO: 77) | (SEQ ID NO: 78) |
| Anti-ASGPR antibody, AGA0008: | AGA0008Ha-G4T1K439E | AGA0008La-KT0.Avi |
| AGA0008Ha-G4T1K439E/AGA0008La-KT0.Avi | (SEQ ID NO: 81) | (SEQ ID NO: 82) |
| Negative control Ab IC17dKn: | IC17HdK-G4T1K439E | IC17L-KT0.Avis |
| IC17HdK-G4T1K439E/IC17L-KT0.Avi | (SEQ ID NO: 87) | (SEQ ID NO: 86) |

TABLE 11

| Bifunctional | | |
|---|---|---|
| antibody | First arm | Second arm |
| 3D8//IC17dKn | 3D8VH-G4T1E356K/hT4VL-KT0.Avi | IC17HdK-G4T1K439E/IC17L-KT0.Avi |
| 3D8//MRA | 3D8VH-G4T1E356K/hT4VL-KT0.Avi | MRAH-G4T1K439E/MRAL-KT0.Avi |
| 3D8//AGA0008 | 3D8VH-G4T1E356K/hT4VL-KT0.Avi | AGA0008Ha-G4T1K439E/AGA0008La-KT0.Avi |
| 2C10//IC17dKn | 2C10VH-G4T1E356K/2C10VL-KT0.Avi | IC17HdK-G4T1K439E/IC17L-KT0.Avi |
| 2C10//MRA | 2C10VH-G4T1E356K/2C10VL-KT0.Avi | MRAH-G4T1K439E/MRAL-KT0.Avi |
| 2C10//AGA0008 | 2C10VH-G4T1E356K/2C10VL-KT0.Avi | AGA0008Ha-G4T1K439E/AGA0008La-KT0.Avi |

### INDUSTRIAL APPLICABILITY

The cytosol-penetrating antigen-binding molecules of the present disclosure can be specifically delivered to the cytosol of target cells, and are useful as antigen-binding molecules for treatment, prevention, diagnosis, or detection.

### SEQUENCE LISTING

1. An antigen-binding molecule comprising a first and a second Fab regions, wherein
(a) the first Fab region binds specifically to a cell surface antigen, and
(b) the second Fab region comprises
(i) a pair of a heavy chain variable region (VH) binding specifically to a cytosolic antigen and a light chain variable region (VL) having cytosol-penetrating ability or
(ii) a pair of a heavy chain variable region (VH) having cytosol-penetrating ability and a light chain variable region (VL) binding specifically to a cytosolic antigen.

2. An antigen-binding molecule comprising a first and a second Fab regions and a single-chain unit, wherein
(a) the first Fab region binds specifically to a cell surface antigen,
(b) the second Fab region has cytosol-penetrating ability, and
(c) the single-chain unit binds specifically to a cytosolic antigen.

3. An antigen-binding molecule comprising a first and a second Fab regions and a single-chain unit, wherein
(a) the first Fab region binds specifically to a cytosolic antigen,
(b) the second Fab region has cytosol-penetrating ability, and
(c) the single-chain unit binds specifically to a cell surface antigen.

4. An antigen-binding molecule comprising a first and a second Fab regions and a single-chain unit, wherein
(a) the first and the second Fab regions comprise
(i) a pair of a heavy chain variable region (VH) binding specifically to a cell surface antigen and a light chain variable region (VL) having cytosol-penetrating ability or
(ii) a pair of a heavy chain variable region (VH) having cytosol-penetrating ability and a light chain variable region (VL) binding specifically to a cell surface antigen, and
(b) the single-chain unit binds specifically to a cytosolic antigen.

5. An antigen-binding molecule comprising a first and a second polypeptide chains, wherein
(a) the first polypeptide chain comprises a first VD1-(X1)n-VD2-C-(X2)n, in which
VD1 is a first heavy chain variable region having cytosol-penetrating ability,
VD2 is a second heavy chain variable region binding specifically to a cell surface antigen,
C is a heavy chain constant region CH1,
X1 is a linker other than CH1,
X2 is an Fc region, and
n is 0 or 1,
(b) the second polypeptide chain comprises a second VD1-(X1)n-VD2-C-(X2)n, in which
VD1 is a first light chain variable region binding specifically to a cytosolic antigen,
VD2 is a second light chain variable region binding specifically to a cell surface antigen,
C is a light chain constant region CL,
X1 is a linker other than CL,
X2 does not comprise an Fc region, and
n is 0 or 1.

6. An antigen-binding molecule comprising a first, a second, and a third Fab regions and an Fc region, wherein
(a) the first Fab region binds specifically to a cell surface antigen,
(b) the second and the third Fab regions comprise
(i) a pair of a heavy chain variable region binding specifically to a cytosolic antigen and a light chain variable region having cytosol-penetrating ability or
(ii) a pair of a heavy chain variable region having cytosol-penetrating ability and a light chain variable region binding specifically to a cytosolic antigen,
(c) the Fc region comprises a first Fc subunit and a second Fc subunit,
(d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

7. An antigen-binding molecule comprising a first, a second, a third, and a fourth Fab regions and an Fc region, wherein
(a) one Fab region selected from the first, the second, the third, and the fourth Fab regions binds specifically to a cell-surface antigen,
(b) three Fab regions other than (a) comprise
(i) a pair of a heavy chain variable region binding specifically to a cytosolic antigen and a light chain variable region having cytosol-penetrating ability, or
(ii) a pair of a heavy chain variable region having cytosol-penetrating ability and a light chain variable region binding specifically to a cytosolic antigen,
(c) the Fc region comprises a first Fc subunit and a second Fc subunit,
(d) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

8. An antigen-binding molecule comprising a region having cytosol-penetrating ability and an Fc region, wherein the Fc region comprises one or more amino acid alterations for enhancing formation of a multimer of the antigen-binding molecule, and wherein the antigen-binding molecule has an elevated cytosol-penetrating ability as compared to an antigen-binding molecule comprising a parent Fc region which does not comprise the one or more amino acid alterations.

9. An antigen-binding molecule comprising a first and a second Fab regions and an Fc region, wherein
(a) the first Fab region binds specifically to a cell surface antigen,
(b) the second Fab region comprises
(i) a pair of a heavy chain variable region (VH) binding specifically to a cytosolic antigen and a light chain variable region (VL) having cytosol-penetrating ability, or
(ii) a pair of a heavy chain variable region (VH) having cytosol-penetrating ability and a light chain variable region (VL) binding specifically to a cytosolic antigen, and
(c) the Fc region comprises one or more amino acid alterations for enhancing formation of a multimer of the antigen-binding molecule.

10. The antigen-binding molecule of any one of claims 1-9, wherein the region having cytosol-penetrating ability does not bind to an antigen expressed on the cell surface that is different from the cell surface antigen.

11. The antigen-binding molecule of any one of claims 1-10, wherein the cell surface antigen is an antigen expressed specifically on a target cell, and wherein the antigen-binding molecule is delivered specifically into cytosol of the target cell.

12. A pharmaceutical composition comprising the antigen-binding molecule of any one of claims 1-11 and a pharmaceutically acceptable carrier.

13. A method of delivering the antigen-binding molecule of any one of claims 1-11 specifically into cytosol of a target cell, wherein the cell surface antigen is an antigen expressed specifically on the target cell.

14. A method of removing, suppressing, or activating a cytosolic antigen in a target cell-specific manner by using the antigen-binding molecule of any one of claims 1-11, wherein the cell surface antigen is an antigen expressed specifically on the target cell.

15. A pharmaceutical composition for diagnosing, preventing, or treating a disease in a subject, wherein a diseased cell expresses the cell surface antigen and the cytosolic antigen to which the antigen-binding molecule of any one of claims 1-11 specifically binds, and wherein the pharmaceutical composition comprises the antigen-binding molecule of any one of claims 1-11 and a pharmaceutically acceptable carrier.
